SEQUENCE LISTING (14 sequences)

SEQ ID NO: 1 - DNA, 1777 bp, synthetic construct
  source 1..1777: mol_type = other DNA
agcaaaagcaggggaaaataaaagcaacaaaaatgaaggcaatactagtagttctgctatatacatttgcaaccgcaaatgcagacacattatgtataggttatcatgcgaacaattcaacagacactgtagacacagtactagaaaagaatgtaacagtaacacactctgttaacctattggaggataagcataacggtaagctatgcaaactgagaggcgtagcaccattgcatctaggtaagtgtaatatagccggatggattctagggaatcccgaatgcgaatcactatcaaccgctagctcatggtcatacatagtcgaaacaccatcaagcgataacggtacatgttatcccggagactttatcgattacgaagagcttagagagcaattgtctagcgtaagctcattcgaaagattcgaaatttttccgaaaactagctcatggcctaatcacgatagtaataaaggcgtaactgccgcatgcccacacgccggagctaaatcattctataagaatctgatttggttagtgaaaaaagggaattcatatccgaaactatctaaatcatacattaacgataagggtaaggaggtactagtgttgtgggggatacaccatccatcaactagcgccgatcagcaatcattgtatcagaacgcagacgcatacgtattcgtagggtctagtagatactctaaaaaatttaaacccgaaatcgcaattagaccgaaagtgagagaccaagagggtagaatgaattactattggacactagtcgaaccaggcgataagattacattcgaagcgacagggaatctagtcgtaccgagatacgcattcgcaatggagagaaacgccggatccggaattattattagcgatacacccgtacacgattgcaatactacatgtcagacaccaaaaggcgcaattaatactagtctgccatttcagaatatacacccaattacaatcggtaagtgtccaaaatacgttaagtcaactaagttgagactcgcaacagggttgagaaatataccgtcaattcaatctagggggttgttcggagcaatcgcagggtttatcgaaggggggtggacaggtatggttgacggatggtacggataccatcatcaaaacgaacagggatccggatacgcagccgatctgaaaagtacacagaacgctatagacgaaattacgaataaagtgaatagcgtaatcgaaaaaatgaatacgcaatttacagccgtaggtaaggagtttaatcatctcgaaaaaaggattgagaatctgaataaaaaagtcgacgacggattcttagacatttggacttataacgccgaactgttagtgttactcgaaaacgaaagaacactagactatcacgattcaaacgttaagaatctatacgaaaaagtgagatcgcaattgaaaaataacgctaaagagatagggaatgggtgtttcgaattctatcataaatgcgataatacatgtatggaatccgttaaaaacggaacatacgattaccctaagtatagcgaagaggctaaactgaatagggaagagatagacggagtgaaactcgaatcaactaggatttatcagatactcgcaatttatagtacggttgccagttcattggtactggtagtctccctgggggcaatcagtttctggatgtgctctaatgggtctctacagtgtagaatatgtatttaacattaggatttcagaagcatgagaaaaacacccttgtttctact

SEQ ID NO: 2 - DNA, 1458 bp, synthetic construct
  source 1..1458: mol_type = other DNA
agcaaaagcaggggtttaaaatgaatccaaaccaaaagataataaccattggttcggtctgtatgacaattggaatggctaacttaatattacaaattggaaacataatctcaatatggattagccactcaatccaattggggaatcagaatcaaatcgaaacatgcaatcaatccgtaattacatacgagaataatacttgggtgaatcagacatacgttaacatatcgaatactaatttcgctgccggacaatccgtcgtgagtgtgaaactagccggtaatagtagtctatgtcccgttagcggatgggctatatactctaaagacaatagcgttagaatcggatctaaaggcgacgtattcgttatacgcgaaccattcataagttgtagtccattagagtgtaggactttttttctgacacagggcgcactattgaacgataagcattctaacggtacaatcaaagataggtcaccatatagaacactaatgtcatgtccgataggcgaagtgcctagtccatacaatagtagattcgaatccgtcgcttggtccgctagcgcatgccatgacggtattaattggttgacaatcgggattagcggacccgataacggcgcagtcgccgtacttaagtataacggtataattaccgatactattaagagttggcgaaataatatattgcgaacacaggaatccgaatgcgcatgcgttaacggatcatgttttaccgttatgactgacggaccatctaacgggcaagcgtcatataagatttttagaatcgaaaaaggtaagatagtgaaatccgtcgaaatgaacgctcctaattatcattacgaagagtgctcatgttatcccgattctagcgaaattacatgcgtatgtagagacaattggcacggatctaatagaccttgggtgtcattcaatcagaatctagagtatcaaatcgggtatatatgctcagggatattcggagacaatcctagacctaacgataagacagggtcatgcggaccagtgagttctaacggcgctaacggcgttaaggggtttagtttcaaatacggtaacggcgtatggatagggagaactaagtcaatctctagtagaaacggattcgaaatgatatgggaccctaacggatggaccggaaccgataataatttttcgattaaacaggatatcgtagggattaacgaatggtcagggtatagcggatcattcgtacagcatccagagttaaccggactcgattgcatacgaccatgtttttgggtcgaactgattagggggagaccgaaagagaatactatatggactagcgggagcagcatatccttttgtggtgtaaacagtgacactgtgggttggtcttggccagacggtgctgagttgccatttaccattgacaagtaatttgttcaaaaaactccttgtttctact

SEQ ID NO: 3 - DNA, 1027 bp, synthetic construct
  source 1..1027: mol_type = other DNA
agcaaaagcaggtagatatttaaagatgagtcttctaaccgaggtcgaaacgtacgttctttctatcatcccgtcaggccccctcaaagccgagatcgcgcagagactggaaagtgtctttgcaggaaagaacacagatcttgaggctctcatggaatggctaaagacaagaccaatcttgtcacctctgactaagggaattttaggatttgtgttcacgctcaccgtgcccagtgagcgaggactgcagcgtagacgctttgtccaaaatgccctaaatgggaatggggacccgaacaacatggatagagcagttaaactatacaagaagctcaaaagagaaataacgttccatggggccaaggaggtgtcactaagctattcaactggtgcacttgccagttgcatgggcctcatatacaacaggatgggaacagtgaccacagaagctgcttttggtctagtgtgtgccacttgtgaacagattgctgattcacagcatcggtctcacagacagatggctactaccaccaatccactaatcaggcatgaaaacagaatggtgctggctagcactacggcaaaggctatggaacagatggctggatcgagtgaacaggcagcggaggccatggaggttgctaatcagactaggcagatggtacatgcaatgagaactattgggactcatcctagctccagtgctggtctgaaagatgaccttcttgaaaatttgcaggcctaccagaagcgaatgggagtgcagatgcagcgattcaagtgatcctctcgtcattgcagcaaatatcattgggatcttgcacctgatattgtggattactgatcgtctttttttcaaatgtatttatcgtcgctttaaatacggtttgaaaagagggccttctacggaaggagtgcctgagtccatgagggaagaatatcaacaggaacagcagagtgctgtggatgttgacgatggtcattttgtcaacatagagctagagtaaaaaactaccttgtttctact

SEQ ID NO: 4 - DNA, 2341 bp, synthetic construct
  source 1..2341: mol_type = other DNA
agcgaaagcaggtcaaatatattcaatatggagagaataaaagaactgagagatctaatgtcgcagtcccgcactcgcgagatactcactaagaccactgtggaccatatggccataatcaaaaagtacacatcaggaaggcaagagaagaaccccgcactcagaatgaagtggatgatggcaatgagatacccaattacagcagacaagagaataatggacatgattccagagaggaatgaacaaggacaaaccctctggagcaaaacaaacgatgctggatcagaccgagtgatggtatcacctctggccgtaacatggtggaataggaatggcccaacaacaagtacagttcattaccctaaggtatataaaacttatttcgaaaaggtcgaaaggttgaaacatggtaccttcggccctgtccacttcagaaatcaagttaaaataaggaggagagttgatacaaaccctggccatgcagatctcagtgccaaggaggcacaggatgtgattatggaagttgttttcccaaatgaagtgggggcaagaatactgacatcagagtcacagctggcaataacaaaagagaagaaagaagagctccaggattgtaaaattgctcccttgatggtggcgtacatgctagaaagagaattggtccgtaaaacaaggtttctcccagtagccggcggaacaggcagtgtttatattgaagtgttgcacttaacccaagggacgtgctgggagcagatgtacactccaggaggagaagtgagaaatgatgatgttgaccaaagtttgattatcgctgctagaaacatagtaagaagagcagcagtgtcagcagacccattagcatctctcttggaaatgtgccacagcacacagattggaggagtaaggatggtggacatccttagacagaatccaactgaggaacaagccgtagacatatgcaaggcagcaatagggttgaggattagctcatctttcagttttggtgggttcactttcaaaaggacaagcggatcatcagtcaagaaagaagaagaagtgctaacgggcaacctccaaacactgaaaataagagtacatgaagggtatgaagaattcacaatggttgggagaagagcaacagctattctcagaaaggcaaccaggagattgatccagttgatagtaagcgggagagacgagcagtcaattgctgaggcaataattgtggccatggtattctcacaggaggattgcatgatcaaggcagttaggggcgatctgaactttgtcaatagggcaaaccagcgactgaaccccatgcaccaactcttgaggcatttccaaaaagatgcaaaagtgcttttccagaactggggaattgaatccatcgacaatgtgatgggaatgatcggaatactgcccgacatgaccccaagcacggagatgtcgctgagagggataagagtcagcaaaatgggagtagatgaatactccagcacggagagagtggtagtgagtattgaccgatttttaagggttagagatcaaagagggaacgtactattgtctcccgaagaagtcagtgaaacgcaaggaactgagaagttgacaataacttattcgtcatcaatgatgtgggagatcaatggccctgagtcagtgctagtcaacacttatcaatggataatcaggaactgggaaattgtgaaaattcaatggtcacaagatcccacaatgttatacaacaaaatggaatttgaaccatttcagtctcttgtccctaaggcaaccagaagccggtacagtggattcgtaaggacactgttccagcaaatgcgggatgtgcttgggacatttgacactgtccaaataataaaacttctcccctttgctgctgccccaccagaacagagtaggatgcaattttcctcattgactgtgaatgtgagaggatcagggttgaggatactggtaagaggcaattctccagtattcaattacaacaaggcaaccaaacgacttacagttcttggaaaggatgcaggtgcattgactgaagatccagatgaaggcacatctggggtggagtctgctgtcctgagaggatttctcattttgggcaaagaagacaagagatatggcccagcattaagcatcaatgaactgagcaatcttgcaaaaggagagaaggctaatgtgctaattgggcaaggggacgtagtgttggtaatgaaacgaaaacgggactctagcatacttactgacagccagacagcgaccaaaagaattcggatggccatcaattagtgtcgaattgtttaaaaacgaccttgtttctact

SEQ ID NO: 5 - DNA, 2341 bp, synthetic construct
  source 1..2341: mol_type = other DNA
agcgaaagcaggcaaaccatttgaatggatgtcaatccgactctacttttcctaaaaattccagcgcaaaatgccataagcaccacattcccttatactggagatcctccatacagccatggaacaggaacaggatacaccatggacacagtaaacagaacacaccaatactcagaaaagggaaagtggacgacaaacacagagactggtgcaccccagctcaacccgattgatggaccactacctgaggataatgaaccaagtgggtatgcacaaacagactgtgttctagaggctatggctttccttgaagaatcccacccaggaatatttgagaattcatgccttgaaacaatggaagttgttcaacaaacaagggtagataaactaactcaaggtcgccagacttatgattggacattaaacagaaatcaaccggcagcaactgcattggccaacaccatagaagtctttagatcgaatggcctaacagctaatgagtcaggaaggctaatagatttcttaaaggatgtaatggaatcaatgaacaaagaggaaatagagataacaacccactttcaaagaaaaaggagagtaagagacaacatgaccaagaagatggtcacgcaaagaacaatagggaagaaaaaacaaagactgaataagagaggctatctaataagagcactgacattaaatacgatgaccaaagatgcagagagaggcaagttaaaaagaagggctatcgcaacacctgggatgcagattagaggtttcgtatactttgttgaaactttagctaggagcatttgcgaaaagcttgaacagtctgggctcccagtagggggcaatgaaaagaaggccaaactggcaaatgttgtgagaaagatgatgactaattcacaagacacagagatttctttcacaatcactggggacaacactaagtggaatgaaaatcaaaatcctcgaatgttcctggcgatgattacatatatcaccagaaatcaacccgagtggttcagaaacatcctgagcatggcacccataatgttctcaaacaaaatggcaagactagggaaagggtacatgttcgagagtaaaagaatgaagattcgaacacaaataccagcagaaatgctagcaagcattgacctgaagtacttcaatgaatcaacaaagaagaaaattgagaaaataaggcctcttctaatagatggcacagcatcactgagtcctgggatgatgatgggcatgttcaacatgctaagtacggtcttgggagtctcgatactgaatcttggacaaaagaaatacaccaagacaatatactggtgggatgggctccaatcatccgacgattttgctctcatagtgaatgcaccaaaccatgagggaatacaagcaggagtggacagattctacaggacctgcaagttagtgggaatcaacatgagcaaaaagaagtcctatataaataagacagggacatttgaattcacaagctttttttatcgctatggatttgtggctaattttagcatggagctacccagctttggagtgtctggagtaaatgaatcagctgacatgagtattggagtaacagtgataaagaacaacatgataaacaatgaccttggacctgcaacggcccagatggctcttcaattgttcatcaaagactacagatacacatataggtgccataggggagacacacaaattcagacgagaagatcatttgagttaaagaagctgtgggatcaaacccaatcaaaagtagggctattagtatcagatggaggaccaaacttatacaatatacggaatcttcacattcctgaagtctgcttaaaatgggagctaatggatgatgattatcggggaagactttgtaatcccctgaatccctttgtcagtcataaagagattgattctgtaaacaatgctgtggtaatgccagcccatggtccagccaaaagcatggaatatgatgccgttgcaactacacattcctggattcccaagaggaatcgttctattctcaacacaagccaaaggggaattcttgaggatgaacagatgtaccagaagtgctgcaatctattcgagaaatttttccctagcagttcatataggagaccggttggaatttctagcatggtggaggccatggtgtctagggcccggattgatgccagggtcgacttcgagtctggacggatcaagaaagaagagttctctgagatcatgaagatctgttccaccattgaagaactcagacggcaaaaataatgaatttaacttgtccttcatgaaaaaatgccttgtttctact

SEQ ID NO: 6 - DNA, 2233 bp, synthetic construct
  source 1..2233: mol_type = other DNA
agcgaaagcaggtactgatccaaaatggaagactttgtgcgacaatgcttcaatccaatgatcgtcgagcttgcggaaaaggcaatgaaagaatatggggaagatccgaaaatcgaaactaacaagtttgctgcaatatgcacacatttggaagtttgtttcatgtattcggatttccatttcatcgacgaacggggtgaatcaataattgtagaatctggtgacccgaatgcactattgaagcaccgatttgagataattgaaggaagagaccgaatcatggcctggacagtggtgaacagtatatgtaacacaacaggggtagagaagcctaaatttcttcctgatttgtatgattacaaagagaaccggttcattgaaattggagtaacacggagggaagtccacatatattacctagagaaagccaacaaaataaaatctgagaagacacacattcacatcttttcattcactggagaggagatggccaccaaagcggactacacccttgacgaagagagcagggcaagaatcaaaactaggcttttcactataagacaagaaatggccagtaggagtctatgggattcctttcgtcagtccgaaagaggcgaagagacaattgaagaaaaatttgagattacaggaactatgcgcaagcttgccgaccaaagtctcccaccgaacttccccagccttgaaaactttagagcctatgtagatggattcgagccgaacggctgcattgagggcaagctttcccaaatgtcaaaagaagtgaacgccaaaattgaaccattcttgaggacgacaccacgccccctcagattgcctgatgggcctctttgccatcagcggtcaaagttcctgctgatggatgctctgaaattaagtattgaagacccgagtcacgagggggagggaataccactatatgatgcaatcaaatgcatgaagacattctttggctggaaagagcctaacatagtcaaaccacatgagaaaggcataaatcccaattacctcatggcttggaagcaggtgctagcagagctacaggacattgaaaatgaagagaagatcccaaggacaaagaacatgaagagaacaagccaattgaagtgggcactcggtgaaaatatggcaccagaaaaagtagactttgatgactgcaaagatgttggagaccttaaacagtatgacagtgatgagccagagcccagatctctagcaagctgggtccaaaatgaattcaataaggcatgtgaattgactgattcaagctggatagaacttgatgaaataggagaagatgttgccccgattgaacatatcgcaagcatgaggaggaactattttacagcagaagtgtcccactgcagggctactgaatacataatgaagggagtgtacataaatacggccttgctcaatgcatcctgtgcagccatggatgactttcagctgatcccaatgataagcaaatgtaggaccaaagaaggaagacggaaaacaaacctgtatgggttcattataaaaggaaggtctcatttgagaaatgatactgatgtggtgaactttgtaagtatggagttctcactcactgacccgagactggagccacacaaatgggaaaaatactgtgttcttgaaataggagacatgctcttgaggactgcgataggccaagtgtcgaggcccatgttcctatatgtgagaaccaatggaacctccaagatcaagatgaaatggggcatggaaatgaggcgctgccttcttcagtctcttcagcagattgagagcatgattgaggccgagtcttctgtcaaagagaaagacatgaccaaggaattctttgaaaacaaatcggaaacatggccaatcggagagtcacccaggggagtggaggaaggctctattgggaaagtgtgcaggaccttactggcaaaatctgtattcaacagtctatatgcgtctccacaacttgaggggttttcggctgaatctagaaaattgcttctcattgttcaggcacttagggacaacctggaacctggaaccttcgatcttggggggctatatgaagcaatcgaggagtgcctgattaatgatccctgggttttgcttaatgcatcttggttcaactccttcctcacacatgcactgaagtagttgtggcaatgctactatttgctatccatactgtccaaaaaagtaccttgtttctact

SEQ ID NO: 7 - DNA, 890 bp, synthetic construct
  source 1..890: mol_type = other DNA
agcaaaagcagggtgacaaaaacataatggactccaacaccatgtcaagctttcaggtagactgtttcctttggcatatccgcaagcgatttgcagacaatggattgggtgatgccccattccttgatcggctccgccgagatcaaaagtccttaaaaggaagaggcaacacccttggcctcgatatcgaaacagccactcttgttgggaaacaaatcgtggaatggatcttgaaagaggaatccagcgagacacttagaatgacaattgcatctgtacctacttcgcggtacctttctgacatgaccctcgaggaaatgtcacgagactggttcatgctcatgcctaggcaaaagataataggccctctttgcgtgcgattggaccaggcgatcatggaaaagaacatagtactgaaagcgaacttcagtgtaatctttaaccgattagagaccttgatactactaagggctttcactgaggagggagcaatagttggagaaatttcaccattaccttctcttccaggacatacttatgaggatgtcaaaaatgcagttggggtcctcatcggaggacttgaatggaatggtaacacggttcgagtctctgaaaatatacagagattcgcttggagaaactgtgatgagaatgggagaccttcactacctccagagcagaaatgaaaagtggcgagagcaattgggacagaaatttgaggaaataaggtggttaattgaagaaatgcggcacagattgaaagcgacagagaatagtttcgaacaaataacatttatgcaagccttacaactactgcttgaagtagaacaagagataagagctttctcgtttcagcttatttaatgataaaaaacacccttgtttctact

SEQ ID NO: 8 - DNA, 1565 bp, synthetic construct
  source 1..1565: mol_type = other DNA
agcaaaagcagggtagataatcactcaatgagtgacatcgaagccatggcgtctcaaggcaccaaacgatcatatgaacaaatggagactggtggggagcgccaggatgccacagaaatcagagcatctgtcggaagaatgattggtggaatcgggagattctacatccaaatgtgcactgaactcaaactcagtgattatgatggacgactaatccagaatagcataacaatagagaggatggtgctttctgcttttgatgagagaagaaataaatacctagaagagcatcccagtgctgggaaggaccctaagaaaacaggaggacccatatatagaagagtagacggaaagtggatgagagaactcatcctttatgacaaagaagaaataaggagagtttggcgccaagcaaacaatggcgaagatgcaacagcaggtcttactcatatcatgatttggcattccaacctgaatgatgccacatatcagagaacaagagcgcttgttcgcaccggaatggatcccagaatgtgctctctaatgcaaggttcaacacttcccagaaggtctggtgccgcaggtgctgcggtgaaaggagttggaacaatagcaatggagttaatcagaatgatcaaacgtggaatcaatgaccgaaatttctggaggggtgaaaatggacgaaggacaagggttgcttatgaaagaatgtgcaatatcctcaaaggaaaatttcaaacagctgcccagagggcaatgatggatcaagtaagagaaagtcgaaacccaggaaacgctgagattgaagacctcattttcctggcacggtcagcactcattctgaggggatcagttgcacataaatcctgcctgcctgcttgtgtgtatgggcttgcagtagcaagtgggcatgactttgaaagggaagggtactcactggtcgggatagacccattcaaattactccaaaacagccaagtggtcagcctgatgagaccaaatgaaaacccagctcacaagagtcaattggtgtggatggcatgccactctgctgcatttgaagatttaagagtatcaagtttcataagaggaaagaaagtgattccaagaggaaagctttccacaagaggggtccagattgcttcaaatgagaatgtggaaaccatggactccaataccctggaactgagaagcagatactgggccataaggaccaggagtggaggaaataccaatcaacaaaaggcatccgcaggccagatcagtgtgcagcctacattctcagtgcagcggaatctcccttttgaaagagcaaccgttatggcagcattcagcgggaacaatgaaggacggacatccgacatgcgaacagaagttataagaatgatggaaagtgcaaagccagaagatttgtccttccaggggcggggagtcttcgagctctcggacgaaaaggcaacgaacccgatcgtgccttcctttgacatgagtaatgaagggtcttatttcttcggagacaatgcagaggagtatgacagttgaggaaaaatacccttgtttctact

SEQ ID NO: 9 - DNA, 1777 bp, synthetic construct
  source 1..1777: mol_type = other DNA
agcaaaagcaggggaaaataaaagcaacaaaaatgaaggcaatactagtagttctgctatatacatttgcaaccgcaaatgcagacacattatgtataggttatcatgcgaacaattcaacagacactgtagacacagtactagaaaagaatgtaacagtaacacactctgttaacctattggaggataagcataacggtaagctatgcaaactgagaggcgtagcaccattgcatctaggtaagtgtaatatagccggatggattctagggaatcccgaatgcgaatcactatcaaccgctagctcatggtcatacatagtcgaaacaccatcaagcgataacggtacatgttatcccggagactttatcgattacgaagagcttagagagcaattgtctagcgtaagctcattcgaaagattcgaaatttttccgaaaactagctcatggcctaatcacgatagtaataaaggcgtaactgccgcatgcccacacgccggagctaaatcattctataagaatctgatttggttagtgaaaaaagggaattcatatccgaaactatctaaatcatacattaacgataagggtaaggaggtactagtgttgtgggggatacaccatccatcaactagcgccgatcagcaatcattgtatcagaacgcagacgcatacgtattcgtagggtctagtagatactctaaaaaatttaaacccgaaatcgcaattagaccgaaagtgagagaccaagagggtagaatgaattactattggacactagtcgaaccaggcgataagattacattcgaagcgacagggaatctagtcgtaccgagatacgcattcgcaatggagagaaacgccggatccggaattattattagcgatacacccgtacacgattgcaatactacatgtcagacaccaaaaggcgcaattaatactagtctgccatttcagaatatacacccaattacaatcggtaagtgtccaaaatacgttaagtcaactaagttgagactcgcaacagggttgagaaatataccgtcaattcaatctagggggttgttcggagcaatcgcagggtttatcgaaggggggtggacaggtatggttgacggatggtacggataccatcatcaaaacgaacagggatccggatacgcagccgatctgaaaagtacacagaacgytatagacgaaattacgaataaagtgaatagcgtaatcgaaaaaatgaatacgcaatttacagccgtaggtaaggagtttaatcatctcgaaaaaaggattgagaatctgaataaaaaagtcgacgacggattcttagacatttggacttataacgccgaactgttagtgttactcgaaaacgaaagaacactagactatcacgattcaaacgttaagaatctatacgaaaaagtgagatcgcaattgaaaaataacgctaaagagatagggaatgggtgtttcgaattctatcataaatgcgataatacatgtatggaatccgttaaaaacggaacatacgattaccctaagtatagcgaagaggctaaactgaatagggaagagatagacggagtgaaactcgaatcaactaggatttatcagatactcgcaatttatagtacggttgccagttcattggtactggtagtctccctgggggcaatcagtttctggatgtgctctaatgggtctctacagtgtagaatatgtatttaacattaggatttcagaagcatgagaaaaacacccttgtttctact

SEQ ID NO: 10 - DNA, 1777 bp, synthetic construct
  source 1..1777: mol_type = other DNA
agcaaaagcaggggaaaataaaagcaacaaaaatgaaggcaatactagtagttctgctatatacatttgcaaccgcaaatgcagacacattatgtataggttatcatgcgaacaattcaacagacactgtagacacagtactagaaaagaatgtaacagtaacacactctgttaacctattggaggataagcataacggtaagctatgcaaactgagaggcgtagcaccattgcatctaggtaagtgtaatatagccggatggattctagggaatcccgaatgcgaatcactatcaaccgctagctcatggtcatacatagtcgaaacaccatcaagcgataacggtacatgttatcccggagactttatcgattacgaagagcttagagagcaattgtctagcgtaagctcattcgaaagattcgaaatttttccgaaaactagctcatggcctaatcacgatagtaataaaggcgtaactgccgcatgcccacacgccggagctaaatcattctataagaatctgatttggttagtgaaaaaagggaattcatatccgaaactatctaaatcatacattaacgataagggtaaggaggtactagtgttgtgggggatacaccatccatcaactagcgccgatcagcaatcattgtatcagaacgcagacgcatacgtattcgtagggtctagtagatactctaaaaaatttaaacccgaaatcgcaattagaccgaaagtgagagaccaagagggtagaatgaattactattggacactagtcgaaccaggcgataagattacattcgaagcgacagggaatctagtcgtaccgagatacgcattcgcaatggagagaaacgccggatccggaattattattagcgatacacccgtacacgattgcaatactacatgtcagacaccaaaaggcgcaattaatactagtctgccatttcagaatatacacccaattacaatcggtaagtgtccaaaatacgttaagtcaactaagttgagactcgcaacagggttgagaaatataccgtcaattcaatctagggggttgttcggagcaatcgcagggtttatcgaaggggggtggacaggtatggttgacggatggtacggataccatcatcaaaacgaacagggatccggatacgcagccgawctgaaaagtacacagaacgytatagacgaaattacgaataaagtgaatagcgtaatcgaaaaaatgaatacgcaatttacagccgtaggtaaggagtttaatcatctcgaaaaaaggattgagaatctgaataaaaaagtcgacgacggattcttagacatttggacttataacgccgaactgttagtgttactcgaaaacgaaagaacactagactatcacgattcaaacgttaagaatctatacgaaaaagtgagatcgcaattgaaaaataacgctaaagagatagggaatgggtgtttcgaattctatcataaatgcgataatacatgtatggaatccgttaaaaacggaacatacgattaccctaagtatagcgaagaggctaaactgaatagggaagagatagacggagtgaaactcgaatcaactaggatttatcagatactcgcaatttatagtacggttgccagttcattggtactggtagtctccctgggggcaatcagtttctggatgtgctctaatgggtctctacagtgtagaatatgtatttaacattaggatttcagaagcatgagaaaaacacccttgtttctact

SEQ ID NO: 11 - DNA, 1777 bp, synthetic construct
  source 1..1777: mol_type = other DNA
agcaaaagcaggggaaaataaaagcaacaaaaatgaaggcaatactagtagttctgctatatacatttgcaaccgcaaatgcagacacattatgtataggttatcatgcgaacaattcaacagacactgtagacacagtactagaaaagaatgtaacagtaacacactctgttaacctattggaggataagcataacagtaagctatgcaaactgagaggcgtagcaccattgcatctaggtaagtgtaatatagccggatggattctagggaatcccgaatgcgaatcactatcaaccgctagctcatggtcatacatagtcgaaacaccatcaagcgataacggtacatgttatcccggagactttatcgattacgaagagcttagagagcaattgtctagcgtaagctcattcgaaagattcgaaatttttccgaaaactagctcatggcctaatcacgatagtagtaaaggcgtaactgccgcatgcccacacgccggagctaaatcattctataagaatctgatttggttagtgaaaaaagagaattcatatccgaaactatctaaatcatacattaacgataagggtaaggaggtactagtgttgtgggggatacaccatccatcaactagcgccgatcagcaatcattgtatcagaacgcagacgcatacgtattcgtagggtctagtagatactctaaaaaatttaaacccgaaatcgcaattagaccgaaagtgagaggccaagagggtagaatgaattactattggacactagtcgaaccaggcgataagattacattcgaagcgacagggaatctagtcgtaccgagatacgcattcgcaatggagagaaacgccggatccggaattattattagcgatacacccgtacacgattgcaatactacgtgtcagacaccaaaaggcgcaattaatactagtctgccatttcagaatatacacccaattacaatcggtaagtgtccaaaatacgttaagtcaactaagttgagactcgcaacagggttgagaaatacaccgtcaattcaatctagggggttgttcggagcaatcgcagggtttatcgaaggggggtggacaggtatggttgacggatggtacggataccatcatcaaaacgaacagggatccggatacgcagccgaactgaaaagtacacagaacgctatagacgaaattacgaataaagtgaatagcgtaatcgaaaaaatgaatacgcaatttacagccgtaggtaaggagtttaatcatctcgaaaaaaggattgagaatctgaataaaaaagtcgacgacggattcttagacatttggacttataacgccgaaatgttagtgttactcgaaaacgaaagaacactagactatcacgattcaaacgttaagaatctatacgaaaaagtgagatcgcaattgaaaaataacgctaaagagatagggaatgggtgtttcgaattctatcataaatgcgataatacatgtatggaatccgttaaaaacggaacatacgattaccctaagtatagcgaagaggctaaactgaatagggaagagatagacggagtggaacttgaatcaactaggatttatcagatactcgcaatttatagtacggttgccagttcattggtactggtagtctccctgggggcaatcagtttctggatgtgctctaatgggtctctacagtgtagaatatgtatttaacattaggatttcagaagcatgagaaaaacacccttgtttctact

SEQ ID NO: 12 - DNA, 1458 bp, synthetic construct
  source 1..1458: mol_type = other DNA
agcaaaagcaggggtttaaaatgaatccaaaccaaaagataataaccattggttcggtctgtatgacaattggaatggctaacttaatattacaaattggaaacataatctcaatatggattagccactcaatccaattggggaatcagaatcaaatcgaaacatgcaatcaatccgtaattacatacgagaataatacttgggtgaatcagacatacgttaacatatcgaatactaatttcactgccggacaatccgtcgtgagtgtgaaactagccggtaatagtagtctatgtcccgttagcggatgggctatatactctaaagacaatagcgttagaatcggatctaaaggcgacgtattcgttatacgcgaaccattcataagttgtagtccattagagtgtaggactttttttctgacacagggcgcactattgaacgataagcattctaacggtacaatcaaagataggtcaccatatagaacactaatgtcatgtccgataggcgaagtgcctagtccatacaatagtagattcgaatccgtcgcttggtccgctagcgcatgccatgacggtattaattggttgacaatcgggattagcggacccgataacggcgcagtcgccgtacttaagtataacggtataattaccgatactattaagagttggcgaaataatatattgcgaacacaggaatccgaatgcgcatgcgttaacggatcatgttttaccgttatgactgacggaccatctaacgggcaagcgtcatataagatttttagaatcgaaaaaggtaagatagtgaaatccgtcgaaatgaacgctcctaattatcattacgaagagtgctcatgttatcccgattctagcgaaattacatgcgtatgtagagacaattggcacggatctaatagaccttgggtgtcattcaatcagaatctagagtatcaaatcgggtatatatgctcagggatattcggagacaatcctagacctaacgataagacagggtcatgcggaccagtgagttctaacggcgctaacggcgttaaggggtttagtttcaaatacggtaacggcgtatggatagggagaactaagtcaatctctagtagaaacggattcgaaatgatatgggaccctaacggatggaccggaaccgataataatttttcgattaaacaggatatcgtagggattaacgaatggtcagggtatagcggatcattcgtacagcatccagagttaaccggactcgattgcatacgaccatgtttttgggtcgaactgattagggggagaccgaaagagaatactatatggactagcgggagcagcatatccttttgtggtgtaaacagtgacactgtgggttggtcttggccagacggtgctgagttgccatttaccattgacaagtaatttgttcaaaaaactccttgtttctact

SEQ ID NO: 13 - DNA, 1777 bp, synthetic construct
  source 1..1777: mol_type = other DNA
agcaaaagcaggggaaaataaaagcaacaaaaatgaaggcaatactagtagttctgctatatacatttgcaaccgcaaatgcagacacattatgtataggttatcatgcgaacaattcaacagacactgtagacacaatactagaaaagaatgtaacagtaacacactctgttaacctattggaggataagcataacggtaagctatgcaaactgagaggcgtagcaccattgcatctaggtaagtgtaatatagccggatggattctagggaatcccgaatgcgaatcactatcaaccgctagctcatggtcatacatagtcgaaacaccatcaagcgataacggtacatgttatcccggagactttatcgattacgaagagcttagagagcaattgtctagcgtaagctcattcgaaagattcgaaatttttccgaaaactagctcatggcctgatcacgatagtaataaaggcgtaactgccgcatgcccacacgccggagctaaatcattctatgagaatctggtttggttagtgaaaaaagggaattcatatccgaaactatctaaatcatacattaacgataagggtaaggaggtactagtgttgtgggggatacaccatccatcaactagcgccgatcagcaatcattgtatcagaacgcagacgcatacgtattcgtagggtctagtagatactctaaaaaatttaaacccgaaatcgcaattagaccgaaagtgagaggccaagagggtagaatgaattactattggacactagtcgaaccaggcgataagattacattcgaagcgacagggaatctagtcgtaccgagatacgcattcgcaatggagagaaacgccggatccggaattattattagcgatacacccgtacacgattgcaatactacatgtcagacaccagaaggcgcaattaatactagtctgccatttcagaatatacacccaattacaatcggtaagtgtccaaaatacgttaagtcaactaagttgagactcgcaacagggttgagaaatataccgtcaattcaatctagggggttgttcggagcaatcgcagggtttatcgaaggggggtggacaggtatggttgacggatggtacggataccatcatcaaaacgaacatggatccggatacgcagccgaactgaaaagtacacagaacgctatagacgaaattacgaataaagtgaatagcgtaatcgaaaaaatgaatacgcaatttacagccgtaggtaaggagtttaatcatctcgaaaaaaggattgagaatctgaataaaaaagtcgacgacggattcttagacatttggacttataacgccgaactgttagtgttactcgaaaacgaaagaacactagactatcacgattcaaacgttaagaatctatacgaaaaagtgagatcgcaattgaaaaataacgctaaagagatagggaatgggtgtttcgaattctatcataaatgcgataatacatgtatggaatccgttaaaaacggaacatacgattaccctaagtatagcgaagaggctaaactgaatagagaagagatagacggagtggaactcgagtcaactaggatttatcagatactcgcaatttatagtacggttgccagttcattggtactggtagtctccctgggggcaatcagtttctggatgtgctctaatgggtctctacagtgtagaatatgtatttaacattaggatttcagaagcatgagaaaaacacccttgtttctact

SEQ ID NO: 14 - DNA, 1458 bp, synthetic construct
  source 1..1458: mol_type = other DNA
agcaaaagcaggggtttaaaatgaatccaaaccaaaagataataaccattggttcggtctgtgtgacaattggaatggctaacttaatattacaaattggaaacataatctcaatatggattagccactcaatccaattggggaatcagaatcaaatcgaaacatgcaatcaatccgtaattacatacgagaataatacttgggtgaatcagacatacgttaacatatcgaatactaatttcactgccggaccatccgtcgtgagtgtgaaactagccggtaatagtagtctatgtcccgttagcggatgggctatatactctaaagacaatagcgttagaatcggatctaaaggcgacgtattcgttatacgcgaaccattcataagttgtagtccattagagtgtaggactttttttctgacacagggcgaactattgaacgataagcattctaacggtacaatcaaagataggtcaccatatagaacactaatgtcatgtccgataggcgaagtgcctagtccatacaatagtaaattcgaatccgtcgcttggtccgctagcgcatgccatgacggtattaattggttgacaatcgggattagcggacccgataacggcgcagtcgccgtacttaagtataacggtataattaccgatactattaagagttggcgaaataatatattgcgaacacaggaatccgaatgcgcatgcgttaacggatcatgttttaccgttatgactgacggaccatctaacgggcaagcgtcatataagatttttagaatcgaaaaaggtaagatagtgaaatccgtcgaaatgaacgctcctaattatcattacgaagagtgctcatgttatcccgattctagcgaaattacatgcgtatgtagagacaattggcacggatctaatagaccttgggtgtcattcaatcagaatctagagtatcaaatcgggtatatatgctcagggatattcggagacaatcctagacctaacgataagacagggtcatgcggaccagtgagttctaacggcgctaacggcgttaaggggtttagtttcaaatacggtaacggcgtatggatagggagaactaagtcaatctctagtagaaacggattcgaaatgatatgggaccctaacggatggaccggaaccgataataatttttcgattaaacaggatatcgtagggattaacgaatggtcagggtatagcggatcattcgtacagcatccagagttaaccggactcgattgcatacgaccatgtttttgggtcgaactgattagggggagaccgaaagagaatactatatggactagcgggagcagcatatccttttgtggtgtaaacagtgacactgtgggttggtcttggccagacggtgctgagttgccatttaccattgacaagtaatttgttcaaaaaactccttgtttctact

(19) United States
(12) Patent Application Publication
(10) Pub. No.: US 2025/0325650 A1
(43) Pub. Date: Oct. 23, 2025

(54) DEOPTIMIZED INFLUENZA VIRUSES AND METHODS OF TREATING CANCER

(71) Applicant: Codagenix Inc., Farmingdale, NY (US)

(72) Inventors: John Robert Coleman, Blauvelt, NY (US); Steffen Mueller, Great Neck, NY (US); Chen Yang, Plainview, NY (US); Ying Wang, South Setauket, NY (US); Charles Stauft, Columbia, MD (US)

(73) Assignee: Codagenix Inc., Farmingdale, NY (US)

(21) Appl. No.: 18/856,367

(22) PCT Filed: Apr. 19, 2023
(86) PCT No.: PCT/US2023/065949

§ 371 Date: Oct. 11, 2024

Related U.S. Application Data
(60) Provisional application No. 63/332,438, filed on Apr. 19, 2022.
(60) Provisional application No. 63/429,652, filed on Dec. 2, 2022.

(51) Int. Cl. A61K 39/145; A61K 39/00; A61K 39/395; A61P 35/00; C07K 16/28; C12N 7/00

(52) U.S. Cl. CPC A61K 39/145; A61K 39/39558; A61P 35/00; C07K 16/2818; C12N 7/00; A61K 2039/507; A61K 2039/5254; A61K 2039/585; C12N 2760/16122; C12N 2760/16134; C12N 2760/16162; C12N 2760/16171

(57) ABSTRACT

The present invention provides for compositions comprising deoptimized influenza viruses and methods of using the composition for the treatment of cancer. Wherein treating a malignant tumor, comprises: administering a deoptimized influenza virus to a subject in need thereof, wherein an HA protein of the deoptimized influenza virus is encoded by a nucleic acid having a specified sequence.

### CROSS-REFERENCE TO RELATED APPLICATIONS

This application includes a claim of priority under 35 U.S.C. § 119(e) to U.S. provisional patent application No. 63/332,438 filed Apr. 19, 2022, and No. 63/429,652 filed Dec. 2, 2022, the entirety of both is hereby incorporated by reference.

### REFERENCE TO SEQUENCE LISTING

This application contains a Sequence Listing submitted as an electronic file named “064955_000067WOPT_Sequence_Listing_ST26”, having a size in bytes of 36,675 bytes, and created on Apr. 19, 2023 (WIPO production date). The information contained in this electronic file is hereby incorporated by reference in its entirety.

### FIELD OF INVENTION

This invention relates to the treatment of cancer with oncolytic deoptimized influenza viruses.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

It has been known that malignant tumors result from the uncontrolled growth of cells in an organ. The tumors grow to an extent where normal organ function may be critically impaired by tumor invasion, replacement of functioning tissue, competition for essential resources and, frequently, metastatic spread to secondary sites. Malignant cancer is the second leading cause of mortality in the United States.

Up to the present, the methods for treating malignant tumors include surgical resection, radiation and/or chemotherapy. However, numerous malignancies respond poorly to all traditionally available treatment options and there are serious adverse side effects to the known and practiced methods. There has been much advancement to reduce the severity of the side effects while increasing the efficiency of commonly practiced treatment regimens. However, many problems remain, and there remains a need to search for alternative modalities of treatment.

### SUMMARY OF THE INVENTION

The following embodiments and aspects thereof are described and illustrated in conjunction with compositions and methods which are meant to be exemplary and illustrative, not limiting in scope.

Various embodiments of the invention provide for a method of treating a malignant tumor, comprising: administering a deoptimized influenza virus to a subject in need thereof, wherein an HA protein of the deoptimized influenza virus is encoded by a nucleic acid having the sequence of SEQ ID NO:1, SEQ ID NO:9, SEQ ID NO:10, SEQ ID NO:13 or ORF of SEQ ID NO:13 or an HA variant of SEQ ID NO:1, SEQ ID NO:9, SEQ ID NO:10, SEQ ID NO:13 or ORF of SEQ ID NO:13 wherein the HA variant does not comprise a nucleic acid having SEQ ID NO:11 or open reading frame (ORF) of SEQ ID NO:11, and wherein an NA protein of the deoptimized influenza virus is encoded by a nucleic acid having the sequence of SEQ ID NO:2, SEQ ID NO:14 or ORF of SEQ ID NO:14 or an NA variant of SEQ ID NO:2, SEQ ID NO:14, or ORF of SEQ ID NO:14 wherein the NA variant does not comprise a nucleic acid having SEQ ID NO:12 or ORF of SEQ ID NO:12.

In various embodiments, the HA protein of the deoptimized influenza virus can be encoded by a nucleic acid having the sequence of SEQ ID NO:9 or SEQ ID NO: 10. In various embodiments, the nucleic acid sequence of the HA variant of SEQ ID NO:1, SEQ ID NO:9 or SEQ ID NO:10 can comprise up to 10 mutations relative to SEQ ID NO:1, SEQ ID NO:9, or SEQ ID NO:10, respectively.

In various embodiments, the nucleic acid sequence of the NA variant of SEQ ID NO:2 can comprise up to 10 mutations relative to SEQ ID NO:2.

In various embodiments, the M, PB2, PB1, PA, NS or NP protein can each be encoded by a nucleic acid having SEQ ID NOs: 3, 4, 5, 6, 7, and 8, respectively, or a M, PB2, PB1, PA, NS or NP variant of SEQ ID NOs: 3, 4, 5, 6, 7, and 8, respectively. In various embodiments, the M, PB2, PB1, PA, NS or NP variant of SEQ ID NOs: 3, 4, 5, 6, 7, and 8, respectively, can each comprise up to 10 mutations relative to SEQ ID NOs: 3, 4, 5, 6, 7, and 8, respectively.

In various embodiments, the variant of SEQ ID NOs: 3, 4, 5, 6, 7, and 8, does not comprise wild-type sequence for encoding M, PB2, PB1, PA NS or NP proteins, respectively.

In various embodiments, the deoptimized influenza virus can be administered intratumorally, subcutaneously, intramuscularly, intradermally, intranasally, or intravenously.

Various embodiments of the invention provide for a method of treating a malignant tumor, comprising: administering a prime dose of a deoptimized influenza virus to a subject in need thereof, wherein an HA protein of the deoptimized influenza virus is encoded by a nucleic acid having the sequence of SEQ ID NO:1, SEQ ID NO:9, SEQ ID NO:10, SEQ ID NO:13 or ORF of SEQ ID NO:13 or an HA variant of SEQ ID NO:1, SEQ ID NO:9, SEQ ID NO:10, SEQ ID NO:13 or ORF of SEQ ID NO:13 wherein the HA variant does not comprise a nucleic acid having SEQ ID NO:11 or open reading frame (ORF) of SEQ ID NO:11, and wherein an NA protein of the deoptimized influenza virus is encoded by a nucleic acid having the sequence of SEQ ID NO:2, SEQ ID NO:14 or ORF of SEQ ID NO:14 or an NA variant of SEQ ID NO:2, SEQ ID NO:14, or ORF of SEQ ID NO:13 wherein the NA variant does not comprise a nucleic acid having SEQ ID NO:12 or ORF of SEQ ID NO: 12; and administering one or more boost dose of the deoptimized influenza virus to the subject in need thereof.

In various embodiments, the HA protein of the deoptimized influenza virus can be encoded by a nucleic acid having the sequence of SEQ ID NO:9 or SEQ ID NO:10. In various embodiments, the nucleic acid sequence of the HA variant of SEQ ID NO:1, SEQ ID NO:9 or SEQ ID NO:10 can comprise up to 10 mutations relative to SEQ ID NO:1, SEQ ID NO:9, or SEQ ID NO:10, respectively.

In various embodiments, the nucleic acid sequence of the NA variant of SEQ ID NO:2 can comprise up to 10 mutations relative to SEQ ID NO:2.

In various embodiments, the M, PB2, PB1, PA, NS or NP protein can each be encoded by a nucleic acid having SEQ ID NOs: 3, 4, 5, 6, 7, and 8, respectively, or a M, PB2, PB1, PA, NS or NP variant of SEQ ID NOs: 3, 4, 5, 6, 7, and 8, respectively. In various embodiments, the M, PB2, PB1, PA, NS or NP variant of SEQ ID NOs: 3, 4, 5, 6, 7, and 8, respectively, can each comprise up to 10 mutations relative to SEQ ID NOs: 3, 4, 5, 6, 7, and 8, respectively.

In various embodiments, the variant of SEQ ID NOs: 3, 4, 5, 6, 7, and 8, does not comprise wild-type sequence for encoding M, PB2, PB1, PA NS or NP proteins, respectively.

In various embodiments, the prime dose can be administered intratumorally, subcutaneously, intramuscularly, intradermally, intranasally, or intravenously.

In various embodiments, the one or more boost dose can be administered intratumorally or intravenously. In various embodiments, a first of the one or more boost dose can be administered about 2 weeks after one prime dose, or if more than one prime dose then about 2 weeks after the last prime dose.

In various embodiments, the prime dose can be administered when the subject does not have cancer.

In various embodiments, the subject can be at a higher risk of developing cancer.

In various embodiments, the one or more boost dose can be administered about every 1, 2, 3, 4, 5, 6, 7, 8, 9 or 10 years after the prime dose when the subject does not have cancer. In various embodiments, the one or more boost dose can be administered after the subject is diagnosed with cancer.

In various embodiments, these methods can further comprise administering a PD-1 inhibitor or a PD-L1 inhibitor. In various embodiments, the PD-1 inhibitor can be an anti-PD1 antibody. In various embodiments, the anti-PD1 antibody can be selected from the group consisting of pembrolizumab, nivolumab, pidilizumab, AMP-224, AMP-514, spartalizumab, cemiplimab, AGEN2034/balstilimab, AK105, BCD-100, BI 754091, JS001, LZM009, MGA012, Sym021, TSR-042/dostarlimab, MGD013, AK104, XmAb20717, tislelizumab, and combinations thereof. In various embodiments, the PD-1 inhibitor can be selected from the group consisting of PF-06801591, anti-PD1 antibody expressing pluripotent killer T lymphocytes (PIK-PD-1), autologous anti-EGFRvIII 4SCAR-IgT cells, and combinations thereof. In various embodiments, the PD-L1 inhibitor can be an anti-PD-L1 antibody. In various embodiments, the anti-PD-L1 antibody can be selected from the group consisting of BGB-A333, CK-301, FAZ053, KN035, MDX-1105, MSB2311, SHR-1316, atezolizumab, avelumab, durvalumab, BMS-936559, CK-301, and combinations thereof. In various embodiments, the anti-PD-L1 inhibitor can be M7824.

In various embodiments, these methods can further comprise administering one or more of chemotherapeutic agent, immunotherapeutic agent, anti-cancer drug, therapeutic viral particle, antimicrobial, cytokine, therapeutic protein, immunotoxin, immunosuppressant, and gene therapeutic.

In various embodiments, treating the malignant tumor can decrease the likelihood of recurrence of the malignant tumor. In various embodiments, treating the malignant tumor can decrease the likelihood of having a second cancer that is different from the malignant tumor. In various embodiments, if the subject develops a second cancer that is different from the malignant tumor, the treatment of the malignant tumor can result in slowing the growth of the second cancer. In various embodiments, after remission of the malignant tumor, if the subject develops a second cancer that is different from the malignant tumor, the treatment of the malignant tumor can result in slowing the growth of the second cancer.

In various embodiments, treating the malignant tumor can stimulate an inflammatory immune response in the tumor. In various embodiments, treating the malignant tumor can recruit pro-inflammatory cells to the tumor. In various embodiments, treating the malignant tumor can stimulate an anti-tumor immune response. In various embodiments, treating the malignant tumor can reduce the tumor size.

In various embodiments, the malignant tumor can be breast cancer, glioblastoma, adenocarcinoma, melanoma, lung carcinoma, neuroblastoma, bladder cancer, colon cancer, prostate cancer, or liver cancer.

Various embodiments of the invention provide for a deoptimized influenza virus, comprising: an HA protein encoded by a nucleic acid having the sequence of SEQ ID NO:1, SEQ ID NO:9, SEQ ID NO:10; SEQ ID NO:13 or ORF of SEQ ID NO:13 or an HA variant of SEQ ID NO:1, SEQ ID NO:9, SEQ ID NO:10, SEQ ID NO:13 or ORF of SEQ ID NO:13 wherein the HA variant does not comprise a nucleic acid having SEQ ID NO:11 or open reading frame (ORF) of SEQ ID NO:11; and an NA protein encoded by a nucleic acid having the sequence of SEQ ID NO:2, SEQ ID NO:14 or ORF of SEQ ID NO:14 or an NA variant of SEQ ID NO:2, SEQ ID NO:14, or ORF of SEQ ID NO:14 wherein the NA variant does not comprise a nucleic acid having SEQ ID NO:12 or ORF of SEQ ID NO:12.

In various embodiments, the HA protein of the deoptimized influenza virus can be encoded by a nucleic acid having the sequence of SEQ ID NO:9 or SEQ ID NO: 10. In various embodiments, the nucleic acid sequence of the HA variant of SEQ ID NO:1, SEQ ID NO:9 or SEQ ID NO:10 can comprise up to 10 mutations relative to SEQ ID NO:1, SEQ ID NO:9, or SEQ ID NO:10, respectively.

In various embodiments, the nucleic acid sequence of the NA variant of SEQ ID NO:2 can comprise up to 10 mutations relative to SEQ ID NO:2.

In various embodiments, the M, PB2, PB1, PA, NS or NP protein can each be encoded by a nucleic acid having SEQ ID NOs: 3, 4, 5, 6, 7, and 8, respectively, or a M, PB2, PB1, PA, NS or NP variant of SEQ ID NOs: 3, 4, 5, 6, 7, and 8, respectively. In various embodiments, the M, PB2, PB1, PA, NS or NP variant of SEQ ID NOs: 3, 4, 5, 6, 7, and 8, respectively, can each comprise up to 10 mutations relative to SEQ ID NOs: 3, 4, 5, 6, 7, and 8, respectively. In various embodiments, the variant of SEQ ID NOs: 3, 4, 5, 6, 7, and 8, does not comprise wild-type sequence for encoding M, PB2, PB1, PA NS or NP proteins, respectively.

Various embodiments provide for a composition comprising the deoptimized influenza virus of the present invention. In various embodiments, the composition can be an immune composition. In various embodiments, the composition can be an oncolytic composition. In various embodiments, the composition comprises about 10⁵-10⁹ PFU of the deoptimized influenza virus.

In various embodiments, the composition can be formulated for parenteral administration. In various embodiments, the composition can be formulated for intratumor administration. In various embodiments, the composition can be formulated for intramuscular injection or subcutaneous injection. In various embodiments, the composition can be formulated for intravenous administration.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts Relative gene expression of sialyltransferase genes ST3Gal1 and ST6Gal1 as well as ERBB2 (encoding Her2) in the breast cancer TCGA data set. Violon plots and quartiles are shown. * p<0.05, *** p<0.001, **** p<0.0001.

FIG. 2 depicts Relative gene expression of sialyltransferase genes ST3Gal1 and ST6Gal1 in Her2^{high} and Her21^{low} breast cancer tissues. Violin plots and quartiles are shown. *** p<0.001, **** p<0.0001.

FIG. 3 depicts gene expression of sialyltransferases ST6Gal1 and ST6Gal2 across multiple tumors (T) and paired normal tissue controls (N) in the TCGA data set. Individual data points and medians are shown on a log 2(TPM+1) scale. Tumor abbreviations (also see Table 1) highlighted in red indicate significant overexpression in tumor vs. normal, abbreviations highlighted in green indicate significantly lower expression in tumor vs. paired normal tissue.

FIG. 4 depicts gene expression of sialyltransferases ST3Gal1 and ST3Gal2 across multiple tumors (T) and paired normal tissue controls (N) in the TCGA data set. Individual data points and medians are shown on a log 2(TPM+1) scale. Tumor abbreviations (also see Table 1) highlighted in red indicate significant overexpression in tumor vs. normal, abbreviations highlighted in green indicate significantly lower expression in tumor vs. paired normal tissue.

FIG. 5 depicts gene expression of sialyltransferases ST3Gal3 and ST3Gal4 across multiple tumors (T) and paired normal tissue controls (N) in the TCGA data set. Individual data point and medians are shown on a log 2(TPM+1) scale. Tumor abbreviations (also see Table 1) highlighted in red indicate significant overexpression in tumor vs. normal, abbreviations highlighted in green indicate significantly lower expression in tumor vs. paired normal tissue.

FIG. 6 depicts gene expression of sialyltransferases ST3Gal5 and ST3Gal6 across multiple tumors (T) and paired normal tissue controls (N) in the TCGA data set. Individual data points and medians are shown on a log 2(TPM+1) scale. Tumor abbreviations (also see Table 1) highlighted in red indicate significant overexpression in tumor vs. normal, abbreviations highlighted in green indicate significantly lower expression in tumor vs. paired normal tissue.

FIG. 7 depicts α2,6 sialic acid expression in breast tissues detected using Sambucus nigra lectin (SNA). Box plots indicating quartiles and individual data points are shown. * p<0.05, * ** p<0.001.

FIG. 8 depicts α2,3 sialic acid expression in breast tissues detected using Maackia amurensis lectin II (MAL2). Box plots indicating quartiles and individual data points are shown. * p<0.05, *** p<0.001.

FIG. 9 depicts initial EMT6 tumor volumes on day 6, equal to the start of treatment, indicating no bias between treatment groups at the start of the experiment. Means and standard deviations are shown.

FIGS. 10A-10C depict EMT6 tumor volumes at various time points after onset of influenza virus A/CA07/09-(HA-NA)^{Min} treatment. Animals are shown individually (10A and 10B) or are summarized across the treatment group using means and standard deviations.

FIG. 11 depicts survival of influenza virus A/CA07/09-(HA-NA)^{Min} and control-treated mice with EMT6 tumors.

FIG. 12 depicts change in body weight after rechallenge in naïve control mice and long-term survivors of EMT6 orthotopic tumors previously treated with influenza virus A/CA07/09-(HA-NA)^{Min}. * p<0.05.

FIGS. 13A and 13B depict EMT6 tumor nodules after rechallenge in naïve control mice and long-term survivors of EMT6 orthotopic tumors treated with influenza virus A/CA07/09-(HA-NA)^{Min}. Numbers of tumor nodules are shown in (13A), representative lungs from a long-term survivor (left) and a naïve control mouse (right) are depicted in (13B). ** p<0.01.

FIG. 14 depicts α2,3- and α2,6-linked sialic acid expression on human and mouse TNBC cell lines and MDCK control cells. Means and standard deviations are shown for infected samples. * p<0.05.

FIG. 15 depicts HA surface expression human and mouse TNBC cell lines and MDCK control cells before and after infection with in influenza virus A/CA07/09-(HA-NA)^{Min}. Means and standard deviations are shown for infected samples. * p<0.05.

FIG. 16 depicts relative normalized gene expression of influenza M in 4 human breast cancer and control cell lines. Means and standard deviations are shown.

FIGS. 17A-17C depict housekeeping gene-normalized changes in gene expression in comparison to uninfected controls in cell lines of breast origin. Means and standard deviations of relative fold changes at 6 h (17A) and 24 h (17B) are shown. A heatmap representation of the same data as −ddCt values is depicted in (17C).

FIG. 18 depicts housekeeping gene-normalized changes in gene expression in comparison to uninfected controls in MRC5 fibroblasts. A heatmap representation of mean −ddCt values is shown at the same color scale as in FIG. 17C for comparison. Values outside of this scale are left blank.

FIG. 19 depicts initial EMT6 volumes on day 6, equal to the start of treatment. Means and standard deviations are shown.

FIGS. 20A-20C depict tumor volumes at various time points after onset of treatment with influenza virus A/CA07/09-(HA-NA)^{Min}. Means and standard deviations are shown at day 17 (20A) or day 27 (20B) with statistical significance calculated by one-way ANOVA. Means and standard deviations are shown over time in (20C) with statistical significance calculated using a mixed effects analysis. * p<0.05, ** p<0.01, *** p<0.001 for comparisons as indicated or in light blue to compare both groups with early secondary tumor implantation, in dark blue to compare both groups with late secondary tumor implantation, in dark gray for A/CA07/09-(HA-NA)^{Min}, E vs control L, and in light gray for A/CA07/09-(HA-NA)^{Min} L vs control E.

FIG. 21 depicts tumor volumes of individual secondary flank tumors over time.

FIG. 22 depicts survival of mice treated with influenza virus A/CA07/09-(HA-NA)M and control. * p<0.05, **** p<0.0001 in light or dark blue to compare for comparisons to matched time point controls (light blue vs control E, dark blue vs control L) and in light or dark gray for comparisons to the non-matching time point control (light gray vs control E, dark gray vs control L).

FIG. 23 depicts ex vivo IFNg recall response to EMT6 tumor cell lysate as quantified by ELISpot. Individual values and means are shown.

FIG. 24 depicts comparison of EMT6 tumor volumes in mice treated with influenza virus A/CA07/09-(HA-NA)^{Min} or control. Means and standard deviations are shown.

FIG. 25 depicts individual EMT6 tumor sizes in mice treated with control (left) or influenza virus A/CA07/09-(HA-NA)^{Min} (right). Each line represents one animal.

FIG. 26 depicts expression of antiviral genes. -dCt values for each individual animal and means are shown.

FIG. 27 depicts expression of chemokine and receptor genes. -dCt values for each individual animal and means are shown. * p<0.05.

FIG. 28 depicts expression of genes associated with anti-tumor immune responses. -dCt values for each individual animal and means are shown. * p<0.05.

FIG. 29 depicts expression of immuno-suppressive genes. -dCt values for each individual animal and means are shown. * p<0.05.

FIG. 30 depicts initial EMT6 tumor volumes on day 6, equal to the start of treatment. Means and standard deviations are shown.

FIGS. 31A-31B depict EMT6 tumor volumes at various time points after onset of influenza virus A/CA07/09-(HA-NA)^{Min} treatment. Means and standard deviations are shown over time (31A) or at day 20 (31B) with statistical significance calculated using two-way or one-way ANOVA with Tukey's multiple comparison test, respectively. Significance values are indicated in the color matching the treatment group in comparison to control treatment. * p<0.05, ** p<0.01, ***p<0.001, **** p<0.0001.

FIG. 32 depicts survival of influenza virus A/CA07/09-(HA-NA)^{Min} and control-treated mice with EMT6 tumors. The frequency of surviving animals is shown over time. *** p<0.001 vs control.

FIG. 33 depicts comparison of EMT6 tumor volumes in control-treated and influenza virus A/CA07/09-(HA-NA)^{Min}-treated mice. Means and standard deviations are shown. Statistical significance was calculated using two-way ANOVA with Geisser-Greenhouse correction and Tukey's multiple comparisons test. Significance is indicated in the color matching the treatment group in comparison to control treatment. * p<0.05, ** p<0.01.

FIG. 34 depicts total immune cell infiltrate in animals treated with influenza virus A/CA07/09-(HA-NA)^{Min} or control media. Frequencies in individual animals and means are shown. Black asterisks indicate statistical significance levels of influenza virus A/CA07/09-(HA-NA)^{Min} vs control, light blue hashes indicate statistical significance levels when comparing the two dose groups of influenza virus A/CA07/09-(HA-NA)^{Min}. ##p<0.01, *** p<0.001.

FIG. 35 depicts lymphoid immune cell infiltrate in control and influenza virus A/CA07/09-(HA-NA)^{Min}-treated tumors. Frequencies as percent of live tumor-infiltrating leukocytes (TIL) in individual animals and means are shown. Black asterisks indicate statistical significance levels of influenza virus A/CA07/09-(HA-NA)^{Min} vs control, light blue hashes indicate statistical significance levels when comparing the two dose groups of influenza virus A/CA07/09-(HA-NA)^{Min}. */#p<0.05.

FIG. 36 depicts myeloid immune cell infiltrate in control and influenza virus A/CA07/09-(HA-NA)^{Min}-treated tumors. Frequencies as percent of live tumor-infiltrating leukocytes (TIL) in individual animals and means are shown. Black asterisks indicate statistical significance levels of influenza virus A/CA07/09-(HA-NA)^{Min} vs control. * p<0.05, ** p<0.001.

FIG. 37 depicts dendritic cell (DC) infiltrate in control and influenza virus A/CA07/09-(HA-NA)^{Min}-treated tumors. Frequencies as percent of live tumor-infiltrating leukocytes (TIL) in individual animals and means are shown.

FIG. 38 depicts ex vivo IFNγ recall response to EMT6 tumor cell lysate as quantified by ELISpot. Individual ratios and means are shown. One way ANOVA with Tukey's multiple comparisons test, * p<0.05, ** p<0.01.

FIG. 39 depicts ex vivo IFNγ recall response to influenza A/CA07/09-(HA-NA)^{Min} as quantified by ELISpot. Individual ratios and means are shown. One way ANOVA with Tukey's multiple comparisons test, *** p<0.001, **** p<0.0001.

FIG. 40 depicts ex vivo IFNγ response to uninfected MDCK.2 cell lysate as quantified by ELISpot. Individual ratios and means are shown. One way ANOVA with Tukey's multiple comparisons test, ** p<0.01, *** p<0.001.

FIG. 41 depicts analysis of gene expression from the tumors that received single treatment with Influenza virus A/CA07/09-(HA-NA)^{Min}. 12 h post treatment all the viral genes were detected above the background levels observed in control-treated samples.

FIG. 42 shows transcriptional upregulation of signaling pathways related to T and B cell function and antigen presentation in mice that received 5 injections of Influenza virus A/CA07/09-(HA-NA)^{Min}. Shades of black to red correspond to the pathways that were upregulated (directed pathway scores >0), while shades of black to green indicate pathways that became downregulated as a result of treatment (directed pathways scores <0).

FIG. 43 shows the combination benefit of CodaLytic with PD-1 checkpoint inhibition in the MC38 CRC model, in which neither monotherapy showed efficacy based on tumor growth (left) and survival (right).

FIG. 44 shows the local recruitment of effector immune cells in the tumor after treatment in the =MC38 CRC model. Increases in infiltration with GranzymeB-expressing cytotoxic T cells was driven by CodaLytic (right), while to total immune infiltrate (CD45, left) and total T cell infiltrate (middle left) were increased more extensively after combination therapy with CodaLytic+PD-1 inhibition.

FIGS. 45A-45C show the results of CodaLytic in therapy of the orthotopically implanted 4T1 mammary carcinoma with anti-PD1 and anti-CTLA4 antibodies.

FIGS. 46A-4CB show the results of an additional experimental set relating to CodaLytic in therapy of 4T1 mammary carcinoma with anti-PD1 and anti-CTLA4 antibodies, including additional experimental control groups.

FIG. 47A shows preferable infection of two breast cancer cell lines as compared to immune cells by CodaLytic, when infected during co-culture. This cell mix mimics different cell types encountered in the tumor microenvironment after intratumoral injection of the virus.

FIG. 47B shows that CodaLytic preferably kills tumor cells over immune cells. CD45+ broadly marks immune cells; CD45− marks non-immune cells, which in this coculture system equate to tumor cells. For CD45+ cells, dead cell percentage did not increase with increasing MOIs and over time. For CD45− cells, dead cell percentage increased along with increasing MOIs and over time. MDA-MB-231 alone showed 16.3% dead cell % without CodaLytic and 45.6% dead cell % at 24 h and MOI 10.

FIGS. 48A-B show the efficacy of CodaLytic alone an in combination with a PD-1 checkpoint inhibition in B16-F10 melanoma. 10≡B16-F10 cells were implanted sq in flanks of C57B1I/6 mice. CodaLytic was administered i.t., three time a week for up to 4 weeks, and 200 ug/dose aPD-1 inhibitor (clone RMP1-14) was administered i.p, two times a week for the same time period. Significant tumor growth inhibition was observed with CodaLytic alone (FIG. 48A), which was further improved upon by addition of aPD-1 checkpoint inhibitor. This translated to improved survival, shown in FIG. 48B.

FIG. 49 shows anti-tumor efficacy and survival in the s.q. CT26 model (n=13). For tumor growth (left panel; top line is vehicle, bottom line is CodaLytic), means and standard deviations are shown until the first study day an animal had to be euthanized. Survival is shown as Kaplan-Meier curves (right panel, top line is CodaLytic, bottom line is vehicle Log-rank test ** p<0.01 vs Vehicle (control)).

FIG. 50A shows the efficacy of CodaLytic in primary human tumoroid cultures, in which the natural human tumor microenvironment is present without external addition of immune cells. Tumoroids form 6 patient tissues (4 breast cancer, 2 renal cell cancer) were treated with CodaLytic, the PD-1 inhibitor pembrolizumab, their combination or respective vehicle controls as indicated in the bottom left. Tumor cell killing was assessed by confocal microscopy and is shown after treatment relative to Vehicle+ Isotype control treatment for each tissue specimen. 25% cell killing is considered meaningful cytotoxicity in this model system.

FIG. 50B shows relative cytokine release 24 h and 48 h after treatment in the same 6 cancer tissues, separated by response as defined by tumor cell killing >25% for each treatment group or in aggregate. Median fold changes vs control are shown.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity.

“Natural isolate” as used herein with reference to influenza virus refers to a virus such as influenza that has been isolated from a host (e.g., human, bird, or any other host) or natural reservoir. The sequence of the natural isolate can be identical or have mutations that arose naturally through the virus' replication cycles as it replicates in and/or transmits between hosts, for example, humans.

“Parent virus” as used herein refer to a reference virus to which a recoded nucleotide sequence is compared for encoding the same or similar amino acid sequence.

“Frequently used codons” or “codon usage bias” as used herein refer to differences in the frequency of occurrence of synonymous codons in coding nucleic acid for a particular species.

“Codon pair bias” as used herein refers to synonymous codon pairs that are used more or less frequently than statistically predicted in a particular species, for example, human, influenza.

“Deoptimized” as used herein with respect to the viruses refer to modified viruses in which their genome, in whole or in part, has synonymous codons and/or codon rearrangements and/or variation of codon pair bias. The substitution of synonymous codons alters various parameters, including for example, codon bias, codon pair bias, density of deoptimized codons and deoptimized codon pairs, RNA secondary structure, CpG dinucleotide content, C+G content, UpA dinucleotide content, translation frameshift sites, translation pause sites, the presence or absence of tissue specific microRNA recognition sequences, or any combination thereof, in the genome.

Mutations described herein are typically synonymous mutations that do not change the resulting amino acid sequence. In some embodiments, the mutation is a nonsynonymous mutation resulting in a change in the amino acid sequence.

A “subject” as used herein means any animal or artificially modified animal. Animals include, but are not limited to, humans, non-human primates, cows, horses, sheep, pigs, dogs, cats, rabbits, ferrets, rodents such as mice, rats and guinea pigs, bats, snakes, and birds. Artificially modified animals include, but are not limited to, SCID mice with human immune systems. In a preferred embodiment, the subject is a human.

A “viral host” means any animal or artificially modified animal that a virus can infect. Animals include, but are not limited to, humans, non-human primates, cows, horses, sheep, pigs, dogs, cats, rabbits, ferrets, rodents such as mice, rats and guinea pigs, and birds. Artificially modified animals include, but are not limited to, SCID mice with human immune systems. In various embodiments, the viral host is a mammal. In various embodiments, the viral host is a primate. In various embodiments, the viral host is human. Embodiments of birds are domesticated poultry species, including, but not limited to, chickens, turkeys, ducks, and geese.

A “prophylactically effective dose” is any amount of a vaccine or virus composition that, when administered to a subject, particularly a subject having a higher risk of cancer, induces in the subject an immune response that protects the subject from developing cancer, or stimulates the immune response in the subject such that if the subject develop cancer at a later time, the effectiveness of a treatment dose can be increased. This includes prevention of recurrence of tumors after initial cure in the adjuvant or neoadjuvant setting. “Protecting” the subject means lessening the likelihood of the disorder's onset in the subject, by at least two-fold, preferably at least ten-fold, 25-fold, 50-fold, or 100 fold. For example, if a subject has a 1% chance of developing cancer, a two-fold reduction in the likelihood of the subject developing cancer would result in the subject having a 0.5% chance of developing cancer.

As used herein, a “therapeutically effective dose” is any amount of a vaccine or virus composition that, when administered to a subject afflicted with a disorder against which the vaccine is effective, induces in the subject an immune response that causes the subject to experience a reduction, remission or regression of the disorder and/or its symptoms. In preferred embodiments, recurrence of the disorder and/or its symptoms is prevented. In other preferred embodiments, the subject is cured of the disorder and/or its symptoms.

“CodaLytic” as used herein in this patent application refers to a deoptimized influenza virus lot made from “A/California/07/2009-(HA-NA)^{Min} having HA, NA, M, PB2, PB1, PA, NS, NP proteins encoded by SEQ ID NOs: 9, 2, 3, 4, 5, 6, 7, 8, respectively. “A/California/07/2009-(HA-NA)^{Min} are deoptimized influenza viruses based on the wild-type sequence of Influenza A virus A/California/07/2009 (also abbreviated as “A/CA07/09”.

Discussed herein, one element of tumor specificity for the deoptimized influenza virus to specific cancer types is related to overexpression of attachment and entry receptors, i.e. surface glycoproteins with terminal sialic acid(s). These viral receptors are required for infection with the deoptimized influenza viruses and compositions. Sialyltransferase expression serves as a surrogate for sialic acid exposure on the cell surface, when sialic acids cannot be detected directly. Sialyltransferase overexpression is found in breast cancer and other tumor types, for example, noted in Example 2 herein. Accordingly, the presently disclosed deoptimized influenza viruses will be effective in the treatment of these sialyltransferase expressing/overexpressing tumors, among others.

### Deoptimized Influenza Compositions

Various embodiments of the present invention provide for a composition comprising a deoptimized influenza virus, wherein the deoptimized virus comprises an HA protein encoded by a nucleic acid having the sequence of SEQ ID NO:1, SEQ ID NO:9, or SEQ ID NO:10 or an HA variant of SEQ ID NO:1, SEQ ID NO:9, or SEQ ID NO:10 wherein the HA variant does not comprise a nucleic acid having SEQ ID NO:11; and an NA protein encoded by a nucleic acid having the sequence of SEQ ID NO:2 or an NA variant of SEQ ID NO:2, wherein the NA variant does not comprise a nucleic acid having SEQ ID NO: 12. In various embodiments, the HA variant does not comprise the open reading frame (ORF) of SEQ ID NO: 11. In various embodiments, the NA variant does not comprise the open reading frame (ORF) of SEQ ID NO: 12.

In various embodiments, the HA protein of the deoptimized influenza virus is encoded by a nucleic acid having the sequence of SEQ ID NO:9 or SEQ ID NO:10.

In various embodiments, the HA protein of the deoptimized influenza virus is encoded by a nucleic acid having the sequence of SEQ ID NO:1. In various embodiments, the HA protein of the deoptimized influenza virus is encoded by a nucleic acid having the sequence of SEQ ID NO:9. In various embodiments, the HA protein of the deoptimized influenza virus is encoded by a nucleic acid having the sequence of SEQ ID NO:10.

In various embodiments the nucleic acid sequence of the HA variant of SEQ ID NO:1, SEQ ID NO:9 or SEQ ID NO:10 does not comprise the wild-type sequence for encoding the HA protein. For example, it does not include the wild-type HA encoding sequence from Influenza A virus A/California/07/2009. In various embodiments the nucleic acid sequence of the HA variant of SEQ ID NO:1, SEQ ID NO:9 or SEQ ID NO: 10 comprises up to 20 mutations relative to SEQ ID NO:1, SEQ ID NO:9, or SEQ ID NO:10, respectively. In various embodiments, HA variant comprises up to 10 mutations. In various embodiments, HA variant comprises up to 5 mutations. In various embodiments, HA variant comprises up to 4, 3, 2, or 1 mutation.

In various embodiments, the Y in SEQ ID NO:9 is C or T. In various embodiments, the Y in SEQ ID NO:9 is C. In various embodiments, the Y in SEQ ID NO:9 is T. In various embodiments, the Y in SEQ ID NO:10 is C. In various embodiments, the Y in SEQ ID NO: 10 is T. In various embodiments, the W in SEQ ID NO:10 is A or T. In various embodiments, the W in SEQ ID NO:10 is A. In various embodiments, the W in SEQ ID NO:10 is T.

In various embodiments the nucleic acid sequence of the NA variant of SEQ ID NO:2 does not comprise the wild-type sequence for encoding the NA protein. For example, it does not include the wild-type NA encoding sequence from Influenza A virus A/California/07/2009. In various embodiments the nucleic acid sequence of the NA variant of SEQ ID NO:2 comprises up to 20 mutations relative to SEQ ID NO:2. In various embodiments, NA variant comprises up to 10 mutations. In various embodiments, NA variant comprises up to 5 mutations. In various embodiments, NA variant comprises up to 4, 3, 2, or 1 mutation.

In various embodiments, the M, PB2, PB1, PA, NS or NP protein are each encoded by a nucleic acid having SEQ ID NOs: 3, 4, 5, 6, 7, and 8, respectively, or a variant of M, PB2, PB1, PA, NS or NP variant of SEQ ID NOs: 3, 4, 5, 6, 7, and 8, respectively. In various embodiments, the M, PB2, PB1, PA, NS or NP protein are each encoded by its corresponding nucleic acid sequence from wild-type A/California/07/2009.

In various embodiments the M, PB2, PB1, PA, NS or NP variant of SEQ ID NOs: 3, 4, 5, 6, 7, and 8, respectively, each comprises up to 20 mutations relative to SEQ ID NOs: 3, 4, 5, 6, 7, and 8, respectively. In various embodiments, the M, PB2, PB1, PA, NS or NP variant comprises up to 10 mutations relative to SEQ ID NOs: 3, 4, 5, 6, 7, and 8, respectively. In various embodiments, the M, PB2, PB1, PA, NS or NP variant comprises up to 5 mutations relative to SEQ ID NOs: 3, 4, 5, 6, 7, and 8, respectively. In various embodiments, the M, PB2, PB1, PA, NS or NP variant comprises up to 4, 3, 2, or 1 mutation relative to SEQ ID NOs: 3, 4, 5, 6, 7, and 8, respectively.

In various embodiments the M, PB2, PB1, PA, NS or NP variant of SEQ ID NOs: 3, 4, 5, 6, 7, and 8, does not comprise wild-type sequence for encoding M, PB2, PB1, PA NS or NP proteins, respectively.

Various embodiments of the present invention provide for a composition comprising a deoptimized influenza virus, wherein the deoptimized virus comprises an HA protein encoded by a nucleic acid having at least 99% sequence identity to SEQ ID NO:1, SEQ ID NO:9, or SEQ ID NO:10, wherein the HA gene does not comprise a nucleic acid having SEQ ID NO:11; an NA protein encoded by a nucleic acid having at least 99% sequence identity to SEQ ID NO:2, wherein the NA gene does not comprise a nucleic acid having SEQ ID NO: 12; an M, PB2, PB1, PA, NS and NP protein are each encoded by a nucleic acid having at least 99% sequence identity to SEQ ID NOs: 3, 4, 5, 6, 7, and 8, respectively. In various embodiments, the HA gene does not comprise the ORF of SEQ ID NO:11. In various embodiments, the NA gene does not comprise the ORF of SEQ ID NO:12. In various embodiments, the deoptimized virus' genome as a whole does not comprise a wild-type influenza virus genome as a whole.

Various embodiments of the present invention provide for a composition comprising a deoptimized influenza virus, wherein the deoptimized virus comprises an HA protein encoded by a nucleic acid having the sequence of SEQ ID NO:13, ORF of SEQ ID NO:13 or an HA variant of SEQ ID NO:13 or ORF of SEQ ID NO:13, wherein the HA variant does not comprise a nucleic acid having SEQ ID NO:11; and an NA protein encoded by a nucleic acid having the sequence of SEQ ID NO:14, ORF of SEQ ID NO:14 or an NA variant of SEQ ID NO:14 or ORF of SEQ ID NO:14, wherein the NA variant does not comprise a nucleic acid having SEQ ID NO:12. In various embodiments, the HA variant does not comprise the open reading frame of SEQ ID NO:11. In various embodiments, the NA variant does not comprise the ORF of SEQ ID NO:12.

In various embodiments the nucleic acid sequence of the HA variant of SEQ ID NO:13 or ORF of SEQ ID NO:13 does not comprise a wild-type sequence for encoding the HA protein. For example, it does not include the wild-type HA encoding sequence from Influenza A virus A/California/07/2009. In various embodiments the nucleic acid sequence of the HA variant of SEQ ID NO:13 or ORF of SEQ ID NO:13 comprises up to 20 mutations relative to SEQ ID NO:13 or ORF of SEQ ID NO:13. In various embodiments, HA variant comprises up to 10 mutations relative to SEQ ID NO:13 or ORF of SEQ ID NO:13. In various embodiments, HA variant comprises up to 5 mutations. In various embodiments, HA variant comprises up to 4, 3, 2, or 1 mutation relative to SEQ ID NO:13 or ORF of SEQ ID NO:13.

In various embodiments the nucleic acid sequence of the NA variant of SEQ ID NO:14 or ORF of SEQ ID NO:14 does not comprise a wild-type sequence for encoding the NA protein. For example, it does not include the wild-type NA encoding sequence from Influenza A virus A/California/07/2009. In various embodiments the nucleic acid sequence of the NA variant of SEQ ID NO:14 or ORF of SEQ ID NO:14 comprises up to 20 mutations relative to SEQ ID NO:14 or ORF of SEQ ID NO: 14. In various embodiments, NA variant comprises up to 10 mutations relative to SEQ ID NO:14 or ORF of SEQ ID NO:14. In various embodiments, NA variant comprises up to 5 mutations relative to SEQ ID NO:14 or ORF of SEQ ID NO:14. In various embodiments, NA variant comprises up to 4, 3, 2, or 1 mutation relative to SEQ ID NO:14 or ORF of SEQ ID NO:14.

In various embodiments, the M, PB2, PB1, PA, NS or NP protein are each encoded by a nucleic acid having SEQ ID NOs: 3, 4, 5, 6, 7, and 8, respectively, or a variant of M, PB2, PB1, PA, NS or NP variant of SEQ ID NOs: 3, 4, 5, 6, 7, and 8, respectively. In various embodiments, the M, PB2, PB1, PA, NS or NP protein are each encoded by its corresponding nucleic acid sequence from wild-type A/California/07/2009.

In various embodiments the M, PB2, PB1, PA, NS or NP variant of SEQ ID NOs: 3, 4, 5, 6, 7, and 8, respectively, each comprises up to 20 mutations relative to SEQ ID NOs: 3, 4, 5, 6, 7, and 8, respectively. In various embodiments, the M, PB2, PB1, PA, NS or NP variant comprises up to 10 mutations relative to SEQ ID NOs: 3, 4, 5, 6, 7, and 8, respectively. In various embodiments, the M, PB2, PB1, PA, NS or NP variant comprises up to 5 mutations relative to SEQ ID NOs: 3, 4, 5, 6, 7, and 8, respectively. In various embodiments, the M, PB2, PB1, PA, NS or NP variant comprises up to 4, 3, 2, or 1 mutation relative to SEQ ID NOs: 3, 4, 5, 6, 7, and 8, respectively.

In various embodiments the M, PB2, PB1, PA, NS or NP variant of SEQ ID NOs: 3, 4, 5, 6, 7, and 8, does not comprise wild-type sequence for encoding M, PB2, PB1, PA NS or NP proteins, respectively.

Various embodiments of the present invention provide for a composition comprising a deoptimized influenza virus, wherein the deoptimized virus comprises an HA protein encoded by a nucleic acid having at least 99% sequence identity to SEQ ID NO:13 or ORF of SEQ ID NO:13, wherein the HA gene does not comprise a nucleic acid having SEQ ID NO:11; an NA protein encoded by a nucleic acid having at least 99% sequence identity to SEQ ID NO:14 or ORF of SEQ ID NO:14, wherein the NA gene does not comprise a nucleic acid having SEQ ID NO:12; an M, PB2, PB1, PA, NS and NP protein are each encoded by a nucleic acid having at least 99% sequence identity to SEQ ID NOs: 3, 4, 5, 6, 7, and 8, respectively. In various embodiments, the HA gene does not comprise the ORF of SEQ ID NO: 11. In various embodiments, the NA gene does not comprise the ORF of SEQ ID NO:12. In various embodiments, the deoptimized virus' genome as a whole does not comprise a wild-type influenza virus genome as a whole.

In various embodiments, mutations comprise a synonymous substitution. That is, the amino acid remains the same. In various embodiments the one or more mutations comprise a nonsynonymous substitution. That is, the mutation results in a change in the amino acid. In various embodiments wherein more than one mutation occurs, the mutations can be all synonymous substitutions, all nonsynonymous substitutions, or both.

In various embodiments, the composition comprises about 10⁵-10⁹ PFU of the deoptimized influenza virus. In various embodiments, the composition comprises about 10⁶-10⁸ PFU of the deoptimized influenza virus. In various embodiments, the composition comprises about 10⁷-10⁸ PFU of the deoptimized influenza virus. In various embodiments, the composition comprises about 10⁶ PFU of the deoptimized influenza virus. In various embodiments, the composition comprises about 10⁷ PFU of the deoptimized influenza virus. In various embodiments, the composition comprises about 10⁸ PFU of the deoptimized influenza virus. In various embodiments, the composition comprises about 5×10⁸ PFU of the deoptimized influenza virus.

In various embodiments, the composition is formulated for parenteral administration. In various embodiments, the composition is formulated for intravenous administration. In various embodiments, the composition is formulated for intramuscular injection or subcutaneous injection. In various embodiments, the composition is formulated for intratumoral administration.

Various embodiments of the present invention provide for a nucleic acid having the sequence of SEQ ID NO:1, SEQ ID NO:9, or SEQ ID NO:10 (which encodes an HA protein) or a variant of SEQ ID NO:1, SEQ ID NO:9, or SEQ ID NO:10 wherein the variant does not comprise a nucleic acid having SEQ ID NO: 11. In various embodiments, the variant does not comprise the open reading frame (ORF) of SEQ ID NO: 11. In various embodiments, the nucleic acid has the sequence of SEQ ID NO:1. In various embodiments, the nucleic acid has the sequence of SEQ ID NO:9. In various embodiments, the nucleic acid has the sequence of SEQ ID NO:10.

In various embodiments the variant of SEQ ID NO:1, SEQ ID NO:9 or SEQ ID NO:10 does not comprise the wild-type sequence for encoding the HA protein. For example, it does not include the wild-type HA encoding sequence from Influenza A virus A/California/07/2009. In various embodiments the nucleic acid sequence of the variant of SEQ ID NO:1, SEQ ID NO:9 or SEQ ID NO:10 comprises up to 20 mutations relative to SEQ ID NO:1, SEQ ID NO:9, or SEQ ID NO:10, respectively. In various embodiments, the variant comprises up to 10 mutations. In various embodiments, the variant comprises up to 5 mutations. In various embodiments, the variant comprises up to 4, 3, 2, or 1 mutation.

In various embodiments, the Y in SEQ ID NO:9 is C or T. In various embodiments, the Y in SEQ ID NO:9 is C. In various embodiments, the Y in SEQ ID NO:9 is T. In various embodiments, the Y in SEQ ID NO: 10 is C. In various embodiments, the Y in SEQ ID NO:10 is T. In various embodiments, the W in SEQ ID NO:10 is A or T. In various embodiments, the W in SEQ ID NO:10 is A. In various embodiments, the W in SEQ ID NO:10 is T.

Various embodiments of the present invention provide for a nucleic acid having at least 99% sequence identity to SEQ ID NO:1, SEQ ID NO:9, or SEQ ID NO:10, wherein the nucleic acid does not comprise a nucleic acid having SEQ ID NO:11.

Various embodiments of the present invention provide for a nucleic acid having the sequence of SEQ ID NO:13, ORF of SEQ ID NO:13 (which encodes a HA protein) or an HA variant of SEQ ID NO:13 or ORF of SEQ ID NO:13, wherein the variant does not comprise a nucleic acid having SEQ ID NO:11. In various embodiments, the variant does not comprise the open reading frame of SEQ ID NO:11.

In various embodiments the nucleic acid sequence of the variant of SEQ ID NO:13 or ORF of SEQ ID NO:13 does not comprise a wild-type sequence for encoding the HA protein. For example, it does not include the wild-type HA encoding sequence from Influenza A virus A/California/07/2009. In various embodiments the nucleic acid sequence of the variant of SEQ ID NO:13 or ORF of SEQ ID NO:13 comprises up to 20 mutations relative to SEQ ID NO:13 or ORF of SEQ ID NO:13. In various embodiments, variant comprises up to 10 mutations relative to SEQ ID NO:13 or ORF of SEQ ID NO:13. In various embodiments, HA variant comprises up to 5 mutations. In various embodiments, HA variant comprises up to 4, 3, 2, or 1 mutation relative to SEQ ID NO:13 or ORF of SEQ ID NO:13.

Various embodiments of the present invention provide for a nucleic acid having at least 99% sequence identity to SEQ ID NO:13 or ORF of SEQ ID NO:13, wherein the nucleic acid does not comprise a nucleic acid having SEQ ID NO:11. In various embodiments, the nucleic acid does not comprise the ORF of SEQ ID NO:11.

Various embodiments of the present invention provide for a nucleic acid having the sequence of SEQ ID NO:2 (which encodes an NA protein) or a variant of SEQ ID NO:2, wherein the variant does not comprise a nucleic acid having SEQ ID NO:12. In various embodiments, the NA variant does not comprise the open reading frame (ORF) of SEQ ID NO:12.

In various embodiments, the nucleic acid sequence of the variant of SEQ ID NO:2 does not comprise the wild-type sequence for encoding the NA protein. For example, it does not include the wild-type NA encoding sequence from Influenza A virus A/California/07/2009. In various embodiments the nucleic acid sequence of the variant of SEQ ID NO:2 comprises up to 20 mutations relative to SEQ ID NO:2. In various embodiments, variant comprises up to 10 mutations. In various embodiments, NA variant comprises up to 5 mutations. In various embodiments, NA variant comprises up to 4, 3, 2, or 1 mutation.

Various embodiments of the present invention provide for a nucleic acid having at least 99% sequence identity to SEQ ID NO:2, wherein the nucleic acid does not comprise a nucleic acid having SEQ ID NO:12.

Various embodiments of the present invention provide for a nucleic acid having the sequence of SEQ ID NO:14, ORF of SEQ ID NO:14 (which encodes an NA protein) or an variant of SEQ ID NO: 14 or ORF of SEQ ID NO:14, wherein the variant does not comprise a nucleic acid having SEQ ID NO:12. In various embodiments, the NA variant does not comprise the ORF of SEQ ID NO:12.

In various embodiments the nucleic acid sequence of the variant of SEQ ID NO: 14 or ORF of SEQ ID NO:14 does not comprise a wild-type sequence for encoding the NA protein. For example, it does not include the wild-type NA encoding sequence from Influenza A virus A/California/07/2009. In various embodiments the nucleic acid sequence of the variant of SEQ ID NO:14 or ORF of SEQ ID NO:14 comprises up to 20 mutations relative to SEQ ID NO:14 or ORF of SEQ ID NO:14. In various embodiments, variant comprises up to 10 mutations relative to SEQ ID NO:14 or ORF of SEQ ID NO:14. In various embodiments, variant comprises up to 5 mutations relative to SEQ ID NO:14 or ORF of SEQ ID NO:14. In various embodiments, variant comprises up to 4, 3, 2, or 1 mutation relative to SEQ ID NO:14 or ORF of SEQ ID NO:14.

Various embodiments provide for a nucleic acid having at least 99% sequence identity to SEQ ID NO:14 or ORF of SEQ ID NO:14, wherein the nucleic acid does not comprise a nucleic acid having SEQ ID NO:12. In various embodiments, the nucleic acid does not comprise the ORF of SEQ ID NO:12.

Various embodiments provide for a genetic construct comprising the nucleic acid sequences as discussed herein.

### Treatment of Existing Cancers

Various embodiments of the present invention provide for a method of treating a malignant tumor, comprising: administering a deoptimized influenza virus to a subject in need thereof, wherein an HA protein of the deoptimized influenza virus is encoded by a nucleic acid having the sequence of SEQ ID NO:1, SEQ ID NO:9, or SEQ ID NO:10 or an HA variant of SEQ ID NO:1, SEQ ID NO:9, or SEQ ID NO:10 wherein the HA variant does not comprise a nucleic acid having SEQ ID NO:11; and wherein an NA protein of the deoptimized influenza virus is encoded by a nucleic acid having the sequence of SEQ ID NO:2 or an NA variant of SEQ ID NO:2, wherein the NA variant does not comprise a nucleic acid having SEQ ID NO:12. In various embodiments, the HA variant does not comprise the ORF of SEQ ID NO:11. In various embodiments, the NA variant does not comprise the ORF of SEQ ID NO:12.

In various embodiments, the HA protein of the deoptimized influenza virus is encoded by a nucleic acid having the sequence of SEQ ID NO:1. In various embodiments, the HA protein of the deoptimized influenza virus is encoded by a nucleic acid having the sequence of SEQ ID NO:9. In various embodiments, the HA protein of the deoptimized influenza virus is encoded by a nucleic acid having the sequence of SEQ ID NO:10.

In various embodiments the nucleic acid sequence of the HA variant of SEQ ID NO:1, SEQ ID NO:9 or SEQ ID NO:10 does not comprise the wild-type sequence for encoding the HA protein. For example, it does not include the wild-type HA encoding sequence from Influenza A virus A/California/07/2009. In various embodiments the nucleic acid sequence of the HA variant of SEQ ID NO:1, SEQ ID NO:9 or SEQ ID NO:10 comprises up to 20 mutations relative to SEQ ID NO:1, SEQ ID NO:9, or SEQ ID NO:10, respectively. In various embodiments, HA variant comprises up to 10 mutations relative to SEQ ID NO:1, SEQ ID NO:9, or SEQ ID NO:10, respectively. In various embodiments, HA variant comprises up to 5 mutations relative to SEQ ID NO:1, SEQ ID NO:9, or SEQ ID NO:10, respectively. In various embodiments, HA variant comprises up to 4, 3, 2, or 1 mutation relative to SEQ ID NO:1, SEQ ID NO:9, or SEQ ID NO:10, respectively.

In various embodiments, the Y in SEQ ID NO:9 is C or T. In various embodiments, the Y in SEQ ID NO:9 is C. In various embodiments, the Y in SEQ ID NO:9 is T. In various embodiments, the Y in SEQ ID NO:10 is C. In various embodiments, the Y in SEQ ID NO: 10 is T. In various embodiments, the W in SEQ ID NO:10 is A or T. In various embodiments, the W in SEQ ID NO:10 is A. In various embodiments, the W in SEQ ID NO:10 is T.

In various embodiments the nucleic acid sequence of the NA variant of SEQ ID NO:2 does not comprise the wild-type sequence for encoding the NA protein. For example, it does not include the wild-type NA encoding sequence from Influenza A virus A/California/07/2009. In various embodiments the nucleic acid sequence of the NA variant of SEQ ID NO:2 comprises up to 20 mutations relative to SEQ ID NO:2. In various embodiments, NA variant comprises up to 10 mutations relative to SEQ ID NO:2. In various embodiments, NA variant comprises up to 5 mutations relative to SEQ ID NO:2. In various embodiments, NA variant comprises up to 4, 3, 2, or 1 mutation relative to SEQ ID NO:2.

In various embodiments, the M, PB2, PB1, PA, NS or NP protein are each encoded by a nucleic acid having SEQ ID NOs: 3, 4, 5, 6, 7, and 8, respectively, or a variant of M, PB2, PB1, PA, NS or NP variant of SEQ ID NOs: 3, 4, 5, 6, 7, and 8, respectively.

In various embodiments the M, PB2, PB1, PA, NS or NP variant of SEQ ID NOs: 3, 4, 5, 6, 7, and 8, respectively, each comprises up to 20 mutations relative to SEQ ID NOs: 3, 4, 5, 6, 7, and 8, respectively. In various embodiments, the M, PB2, PB1, PA, NS or NP variant comprises up to 10 mutations relative to SEQ ID NOs: 3, 4, 5, 6, 7, and 8, respectively. In various embodiments, the M, PB2, PB1, PA, NS or NP variant comprises up to 5 mutations relative to SEQ ID NOs: 3, 4, 5, 6, 7, and 8, respectively. In various embodiments, the M, PB2, PB1, PA, NS or NP variant comprises up to 4, 3, 2, or 1 mutation relative to SEQ ID NOs: 3, 4, 5, 6, 7, and 8, respectively.

In various embodiments the M, PB2, PB1, PA, NS or NP variant of SEQ ID NOs: 3, 4, 5, 6, 7, and 8, does not comprise a wild-type sequence for encoding M, PB2, PB1, PA NS or NP proteins, respectively.

Various embodiments of the present invention provide for a method of treating a malignant tumor, comprising: administering a deoptimized influenza virus to a subject in need thereof, wherein the deoptimized virus comprises an HA protein encoded by a nucleic acid having the sequence of SEQ ID NO:13 or ORF of SEQ ID NO:13, or an HA variant of SEQ ID NO:13 or ORF of SEQ ID NO:13, wherein the HA variant does not comprise a nucleic acid having SEQ ID NO:11; and an NA protein encoded by a nucleic acid having the sequence of SEQ ID NO:14 or ORF of SEQ ID NO:14 or an NA variant of SEQ ID NO:14 or ORF of SEQ ID NO:14, wherein the NA variant does not comprise a nucleic acid having SEQ ID NO:12. In various embodiments, the HA variant does not comprise the ORF of SEQ ID NO:11. In various embodiments, the NA variant does not comprise the ORF of SEQ ID NO:12.

In various embodiments the nucleic acid sequence of the HA variant of SEQ ID NO: 13 or ORF of SEQ ID NO:13, does not comprise the wild-type sequence for encoding the HA protein. For example, it does not include the wild-type HA encoding sequence from Influenza A virus A/California/07/2009.In various embodiments the nucleic acid sequence of the HA variant of SEQ ID NO:13 or ORF of SEQ ID NO:13 comprises up to 20 mutations relative to SEQ ID NO:13 or ORF of SEQ ID NO:13. In various embodiments, HA variant comprises up to 10 mutations relative to SEQ ID NO:13 or ORF of SEQ ID NO:13. In various embodiments, HA variant comprises up to 5 mutations relative to SEQ ID NO:13 or ORF of SEQ ID NO:13. In various embodiments, HA variant comprises up to 4, 3, 2, or 1 mutation relative to SEQ ID NO: 13 or ORF of SEQ ID NO:13.

In various embodiments the nucleic acid sequence of the NA variant of SEQ ID NO:14 or ORF of SEQ ID NO:14 does not comprise the wild-type sequence for encoding the NA protein. For example, it does not include the wild-type NA encoding sequence from Influenza A virus A/California/07/2009. In various embodiments, the nucleic acid sequence of the NA variant of SEQ ID NO:14 or ORF of SEQ ID NO:14 comprises up to 20 mutations relative to SEQ ID NO:14 or ORF of SEQ ID NO:14. In various embodiments, NA variant comprises up to 10 mutations relative to SEQ ID NO:14 or ORF of SEQ ID NO:14. In various embodiments, NA variant comprises up to 5 mutations relative to SEQ ID NO:14 or ORF of SEQ ID NO:14. In various embodiments, NA variant comprises up to 4, 3, 2, or 1 mutation relative to SEQ ID NO:14 or ORF of SEQ ID NO:14.

In various embodiments, the M, PB2, PB1, PA, NS or NP protein are each encoded by a nucleic acid having SEQ ID NOs: 3, 4, 5, 6, 7, and 8, respectively, or a variant of M, PB2, PB1, PA, NS or NP variant of SEQ ID NOs: 3, 4, 5, 6, 7, and 8, respectively.

In various embodiments the M, PB2, PB1, PA, NS or NP variant of SEQ ID NOs: 3, 4, 5, 6, 7, and 8, respectively, each comprises up to 20 mutations relative to SEQ ID NOs: 3, 4, 5, 6, 7, and 8, respectively. In various embodiments, the M, PB2, PB1, PA, NS or NP variant comprises up to 10 mutations relative to SEQ ID NOs: 3, 4, 5, 6, 7, and 8, respectively. In various embodiments, the M, PB2, PB1, PA, NS or NP variant comprises up to 5 mutations relative to SEQ ID NOs: 3, 4, 5, 6, 7, and 8, respectively. In various embodiments, the M, PB2, PB1, PA, NS or NP variant comprises up to 4, 3, 2, or 1 mutation relative to SEQ ID NOs: 3, 4, 5, 6, 7, and 8, respectively.

In various embodiments the M, PB2, PB1, PA, NS or NP variant of SEQ ID NOs: 3, 4, 5, 6, 7, and 8, does not comprise a wild-type sequence for encoding M, PB2, PB1, PA NS or NP proteins, respectively.

Various embodiments of the present invention provide for a method of treating a malignant tumor, comprising: administering a deoptimized influenza virus to a subject in need thereof, wherein an HA protein of the deoptimized influenza virus is encoded by a nucleic acid having at least 99% sequence identity to SEQ ID NO:1, SEQ ID NO:9, or SEQ ID NO:10, wherein the HA gene does not comprise a nucleic acid having SEQ ID NO:11; an NA protein of the deoptimized influenza virus is encoded by a nucleic acid having at least 99% sequence identity to SEQ ID NO:2, wherein the NA gene does not comprise a nucleic acid having SEQ ID NO:12; an M, PB2, PB1, PA, NS and NP protein of the deoptimized influenza virus are each encoded by a nucleic acid having at least 99% sequence identity to SEQ ID NOs: 3, 4, 5, 6, 7, and 8, respectively. In various embodiments, the HA gene does not comprise the ORF of SEQ ID NO: 11. In various embodiments, the NA gene does not comprise the ORF of SEQ ID NO:12. In various embodiments, the deoptimized virus' genome as a whole does not comprise a wild-type influenza virus genome as a whole.

Various embodiments of the present invention provide for a method of treating a malignant tumor, comprising: administering a deoptimized influenza virus to a subject in need thereof, wherein the deoptimized virus comprises an HA protein encoded by a nucleic acid having at least 99% sequence identity to SEQ ID NO:13 or ORF of SEQ ID NO:13, wherein the HA gene does not comprise a nucleic acid having SEQ ID NO:11; an NA protein encoded by a nucleic acid having at least 99% sequence identity to SEQ ID NO:14 or ORF of SEQ ID NO:14, wherein the NA gene does not comprise a nucleic acid having SEQ ID NO:12; an M, PB2, PB1, PA, NS and NP protein are each encoded by a nucleic acid having at least 99% sequence identity to SEQ ID NOs: 3, 4, 5, 6, 7, and 8, respectively. In various embodiments, the HA gene does not comprise the ORF of SEQ ID NO:11. In various embodiments, the NA gene does not comprise the ORF of SEQ ID NO:12. In various embodiments, the deoptimized virus' genome as a whole does not comprise a wild-type influenza virus genome as a whole.

In various embodiments, mutation comprises a synonymous substitution. That is, the amino acid remains the same. In various embodiments the one or more mutations comprise a nonsynonymous substitution. That is, the mutation results in a change in the amino acid. In various embodiments wherein more than one mutation occurs, the mutations can be all synonymous substitutions, all nonsynonymous substitutions, or both.

In various embodiments, a composition comprising about 10⁵-10⁹ PFU of the deoptimized influenza virus is administered. In various embodiments, a composition comprising about 10⁵-10⁹ PFU of the deoptimized influenza virus is administered intratumorally. In various embodiments, an amount of about 10⁵-10⁹ PFU is administered subcutaneously, intramuscularly, intradermally, intranasally, or intravenously.

In various embodiments, a composition comprising about 10⁶-10⁸ PFU of the deoptimized influenza virus is administered. In various embodiments, a composition comprising about 10⁶-10⁸ PFU of the deoptimized influenza virus is administered intratumorally. In various embodiments, an amount of about 10⁶-10⁸ PFU is administered subcutaneously, intramuscularly, intradermally, intranasally, or intravenously.

In various embodiments, a composition comprising about 10⁷-10⁸ PFU of the deoptimized influenza virus is administered. In various embodiments, a composition comprising about 10⁷-10⁸ PFU of the deoptimized influenza virus is administered intratumorally. In various embodiments, an amount of about 10⁷-10⁸ PFU is administered subcutaneously, intramuscularly, intradermally, intranasally, or intravenously.

In various embodiments, a composition comprising about 10⁶ PFU of the deoptimized influenza virus is administered. In various embodiments, a composition comprising about 10⁷ PFU of the deoptimized influenza virus is administered intratumorally. In various embodiments, an amount of about 10⁸ PFU is administered subcutaneously, intramuscularly, intradermally, intranasally, or intravenously. In various embodiments, an amount of about 5×10⁸ PFU is administered subcutaneously, intramuscularly, intradermally, intranasally, or intravenously.

In various embodiments, the one or more additional doses of the deoptimized influenza virus are administered after the initial dose. In various embodiments, the one or more additional doses of the deoptimized influenza virus are administered every 2-3 days after the initial dose for up to 4 weeks.

In various embodiments, the one or more additional doses of the deoptimized influenza virus are administered every 1-6 weeks after the initial dose for 2-6 total doses. In various embodiments, the one or more additional doses of the deoptimized influenza virus are administered every 2-5 weeks after the initial dose for 2-6 total doses. In various embodiments, the one or more additional doses of the deoptimized influenza virus are administered every 2-4 weeks after the initial dose for 3-5 total doses.

In various embodiments, one or more cycles of the deoptimized influenza virus are administered. For example, after an initial cycle every 2-4 weeks for total of 3-5 doses, a resting period is made before a subsequent cycle of the deoptimized influenza virus are administered. The resting period can be, for example, about 1 month, about 2 months, about 3 months, or about 4 months.

In various embodiments, the method further comprises administering a PD-1 inhibitor or a PD-L1 inhibitor. In various embodiments, the PD-1 inhibitor is an anti-PD1 antibody. In various embodiments, the anti-PD1 antibody is selected from the group consisting of pembrolizumab, nivolumab, pidilizumab, AMP-224, AMP-514, spartalizumab, cemiplimab, AGEN2034/balstilimab, AK105, BCD-100, BI 754091, JS001, LZM009, MGA012, Sym021, TSR-042/dostarlimab, MGD013, AK104, XmAb20717, tislelizumab, and combinations thereof. In various embodiments, the PD-1 inhibitor is selected from the group consisting of PF-06801591, anti-PD1 antibody expressing pluripotent killer T lymphocytes (PIK-PD-1), autologous anti-EGFRvIII 4SCAR-IgT cells, and combinations thereof. In various embodiments, the PD-L1 inhibitor is an anti-PD-L1 antibody. In various embodiments, the anti-PD-L1 antibody is selected from the group consisting of BGB-A333, CK-301, FAZ053, KN035, MDX-1105, MSB2311, SHR-1316, atezolizumab, avelumab, durvalumab, BMS-936559, CK-301, and combinations thereof. In various embodiments, the anti-PD-L1 inhibitor is M7824/bintrafusp alpha.

In various embodiments, the method further comprises administering a chemotherapeutic agent. For example, taxanes (paclitaxel, nab-paclitaxel, docetaxel), platinum based therapies (cisplatin), gemcitabine, doxorubicin, or cyclophosphamide.

Additional examples of chemotherapeutic agent include but are not limited to chemotherapeutic agents include cytotoxic agents (e.g., 5-fluorouracil, cisplatin, carboplatin, methotrexate, daunorubicin, doxorubicin (Adriamycin®), vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic akylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

In various embodiments, the method further comprises administering a cancer immunotherapy; for example, CTLA-4 blockade (e.g. ipilimumab, tremelimumab, zalifrelimab and botensilimab), LAG-3 blockade (e.g. relatlimab, TSR-033/GSK4074386, and LAG525), TIM-3 blockade (e.g. cobolimab/TSR-022/GSK4069889, LY3321367 and sabatolimab/MBG453) and modulators of the CD226/TIGIT axis (including agonists) (e.g. TIGIT-targeting antibodies including but not limited to tiragolumab, vibostolimab/MK-7684, ociperlimab/BGB-A1217, domvanalimab/AB154, BMS-986207, IB1I939, etigilimab and GSK4428859/EOS884448); PVRIG-targeting antibodies including but not limited to COM701 and GSK4381562; CD226-targeting antibodies including but not limited to LY3435151; and CD96-targeting antibodies including but not limited to GSK6097608.

In various embodiments, the method further comprises administration of an additional therapeutic agent. Examples of therapeutic agents that may be used in accordance with various embodiments of the present invention include: anti-cancer drugs (including chemotherapeutic agents and antiproliferative agents), therapeutic viral particles, antimicrobials (e.g., antibiotics, antifuingals, antivirals), cytokines and therapeutic proteins, immunotoxins, immunosuppressants, and gene therapeutics (e.g., adenoviral vectors, adeno-associated viral vectors, retroviral vectors, herpes simplex viral vectors, pox virus vectors).

Examples of antiproliferative agents include alkylating agents, antimetabolites, enzymes, biological response modifiers, hormones and antagonists, androgen inhibitors (e.g., flutamide and leuprolide acetate), antiestrogens (e.g., tamoxifen citrate and analogs thereof, toremifene, droloxifene and roloxifene), Additional examples of antiproliferative agents include, but are not limited to levamisole, gallium nitrate, granisetron, sargramostim strontium-89 chloride, filgrastim, pilocarpine, dexrazoxane, and ondansetron.

In various embodiments, treating the malignant tumor decreases the likelihood of recurrence of the malignant tumor.

In various embodiments, treating the malignant tumor decreases the likelihood of having a second cancer that is different from the malignant tumor.

In various embodiments, if the subject develops a second cancer that is different from the malignant tumor, the treatment of the malignant tumor results in slowing the growth of the second cancer.

In various embodiments, after remission of the malignant tumor, if the subject develops a second cancer that is different from the malignant tumor, the treatment of the malignant tumor results in slowing the growth of the second cancer.

In various embodiments, treating the malignant tumor stimulates an inflammatory immune response in the tumor. In various embodiments, treating the malignant tumor recruits pro-inflammatory cells to the tumor. In various embodiments, treating the malignant tumor stimulates an anti-tumor immune response.

In various embodiments, treating the malignant tumor reduced the tumor size.

In various embodiments, the malignant tumor is breast cancer (including triple negative breast cancer), glioblastoma, adenocarcinoma, melanoma, lung carcinoma, neuroblastoma, bladder cancer, colon cancer, prostate cancer, or liver cancer.

In various embodiments, the malignant tumor is a sialyltransferase expressing or overexpressing tumor. In various embodiments, the sialyltransferase is ST6Gal1. In various embodiments, the sialyltransferase is ST6Gal2. In various embodiments, the sialyltransferase is ST3Gal1, ST3Gal2, ST3Gal4, ST3Gal6, or combinations thereof.

In various embodiments, the malignant tumor is testicular germ cell tumors (TGCT), diffuse large B cell lymphoma (DLBC), pancreatic adenocarcinoma (PAAD) and ovarian serous cystadenocarcinoma (OV), skin cutaneous melanoma (SKCM), tumors of the gastrointestinal tract (stomach (STAD), rectal (READ), colon (COAD), and esophageal (ESCA) carcinomas), lower grade glioma (LGG) and glioblastoma (GBM), thymoma (THYM), or hepatocellular carcinoma (LIHC).

In various embodiments, the malignant tumor is pancreatic adenocarcinoma (PAAD) or melanoma (SKCM).

### Prime-Boost Treatments

Various embodiments of the present invention provide for a method of eliciting an immune response and inducing an oncolytic effect on a tumor or cancer cell, using a “prime-boost” type treatment regimen. In various embodiments, eliciting the immune response and inducing an oncolytic effect on the tumor or cancer cell results in treating a malignant tumor.

A “prime” (first) dose of an attenuated virus or a modified virus of the present invention is administered to elicit an initial immune response. Thereafter, one or more boost (subsequent) doses of an attenuated virus or a modified virus of the present invention is administered to induce oncolytic effects on the tumor and/or to elicit an immune response comprising oncolytic effect against the tumor. In some embodiments, the “prime” dose is a smaller dosage than the one or more “boost” doses. In other embodiments, the “prime” dose is about the same dosage amount as the one or more “boost” doses.

Various embodiments of the present invention provide for a method of treating a malignant tumor, comprising: administering a prime dose of a deoptimized influenza virus to a subject in need thereof, wherein an HA protein of the deoptimized influenza virus is encoded by a nucleic acid having the sequence of SEQ ID NO:1, SEQ ID NO:9, or SEQ ID NO:10 or an HA variant of SEQ ID NO:1, SEQ ID NO:9, or SEQ ID NO:10 wherein the HA variant does not comprise a nucleic acid having SEQ ID NO:11 and wherein an NA protein of the deoptimized influenza virus is encoded by a nucleic acid having the sequence of SEQ ID NO:2 or an NA variant of SEQ ID NO:2, wherein the NA variant does not comprise a nucleic acid having SEQ ID NO:12; and administering one or more boost dose of the deoptimized influenza virus to the subject in need thereof. In various embodiments, the HA variant does not comprise the ORF of SEQ ID NO:11. In various embodiments, the NA variant does not comprise the ORF of SEQ ID NO:12.

In various embodiments, the HA protein of the deoptimized influenza virus is encoded by a nucleic acid having the sequence of SEQ ID NO:1. In various embodiments, the HA protein of the deoptimized influenza virus is encoded by a nucleic acid having the sequence of SEQ ID NO:9. In various embodiments, the HA protein of the deoptimized influenza virus is encoded by a nucleic acid having the sequence of SEQ ID NO:10.

In various embodiments the nucleic acid sequence of the HA variant of SEQ ID NO:1, SEQ ID NO:9 or SEQ ID NO:10 does not comprise the wild-type sequence for encoding the HA protein. For example, it does not include the wild-type HA encoding sequence from Influenza A virus A/California/07/2009.In various embodiments the nucleic acid sequence of the HA variant of SEQ ID NO:1, SEQ ID NO:9 or SEQ ID NO:10 comprises up to 20 mutations relative to SEQ ID NO:1, SEQ ID NO:9, or SEQ ID NO:10, respectively. In various embodiments, HA variant comprises up to 10 mutations relative to SEQ ID NO:1, SEQ ID NO:9, or SEQ ID NO:10, respectively. In various embodiments, HA variant comprises up to 5 mutations relative to SEQ ID NO:1, SEQ ID NO:9, or SEQ ID NO:10, respectively. In various embodiments, HA variant comprises up to 4, 3, 2, or 1 mutation relative to SEQ ID NO:1, SEQ ID NO:9, or SEQ ID NO:10, respectively.

In various embodiments, the Y in SEQ ID NO:9 is C or T. In various embodiments, the Y in SEQ ID NO:9 is C. In various embodiments, the Y in SEQ ID NO:9 is T. In various embodiments, the Y in SEQ ID NO:10 is C. In various embodiments, the Y in SEQ ID NO: 10 is T. In various embodiments, the W in SEQ ID NO:10 is A or T. In various embodiments, the W in SEQ ID NO:10 is A. In various embodiments, the W in SEQ ID NO:10 is T.

In various embodiments the nucleic acid sequence of the NA variant of SEQ ID NO:2 does not comprise the wild-type sequence for encoding the NA protein. For example, it does not include the wild-type NA encoding sequence from Influenza A virus A/California/07/2009. In various embodiments the nucleic acid sequence of the NA variant of SEQ ID NO:2 comprises up to 20 mutations relative to SEQ ID NO:2. In various embodiments, NA variant comprises up to 10 mutations relative to SEQ ID NO:2. In various embodiments, NA variant comprises up to 5 mutations relative to SEQ ID NO:2. In various embodiments, NA variant comprises up to 4, 3, 2, or 1 mutation relative to SEQ ID NO:2.

In various embodiments, the M, PB2, PB1, PA, NS or NP protein are each encoded by a nucleic acid having SEQ ID NOs: 3, 4, 5, 6, 7, and 8, respectively, or a variant of M, PB2, PB1, PA, NS or NP variant of SEQ ID NOs: 3, 4, 5, 6, 7, and 8, respectively.

In various embodiments the M, PB2, PB1, PA, NS or NP variant of SEQ ID NOs: 3, 4, 5, 6, 7, and 8, respectively, each comprises up to 20 mutations relative to SEQ ID NOs: 3, 4, 5, 6, 7, and 8, respectively. In various embodiments, the M, PB2, PB1, PA, NS or NP variant comprises up to 10 mutations relative to SEQ ID NOs: 3, 4, 5, 6, 7, and 8, respectively. In various embodiments, the M, PB2, PB1, PA, NS or NP variant comprises up to 5 mutations relative to SEQ ID NOs: 3, 4, 5, 6, 7, and 8, respectively. In various embodiments, the M, PB2, PB1, PA, NS or NP variant comprises up to 4, 3, 2, or 1 mutation relative to SEQ ID NOs: 3, 4, 5, 6, 7, and 8, respectively.

In various embodiments the M, PB2, PB1, PA, NS or NP variant of SEQ ID NOs: 3, 4, 5, 6, 7, and 8, does not comprise wild-type sequence for encoding M, PB2, PB1, PA NS or NP proteins, respectively.

Various embodiments of the present invention provide for a method of treating a malignant tumor, comprising: administering a prime dose of a deoptimized influenza virus to a subject in need thereof, wherein the deoptimized virus comprises an HA protein encoded by a nucleic acid having the sequence of SEQ ID NO:13 or ORF of SEQ ID NO:13, an HA variant of SEQ ID NO:13 or ORF of SEQ ID NO:13, wherein the HA variant does not comprise a nucleic acid having SEQ ID NO:11; and an NA protein encoded by a nucleic acid having the sequence of SEQ ID NO:14 or ORF of SEQ ID NO:14 or an NA variant of SEQ ID NO:14 or ORF of SEQ ID NO:14, wherein the NA variant does not comprise a nucleic acid having SEQ ID NO:12; and administering one or more boost dose of the deoptimized influenza virus to the subject in need thereof. In various embodiments, the HA variant does not comprise the ORF of SEQ ID NO:11. In various embodiments, the NA variant does not comprise the ORF of SEQ ID NO:12.

In various embodiments the nucleic acid sequence of the HA variant of SEQ ID NO:13 or ORF of SEQ ID NO:13, does not comprise the wild-type sequence for encoding the HA protein. For example, it does not include the wild-type HA encoding sequence from Influenza A virus A/California/07/2009. In various embodiments the nucleic acid sequence of the HA variant of SEQ ID NO:13 or ORF of SEQ ID NO:13 comprises up to 20 mutations relative to SEQ ID NO:13 or ORF of SEQ ID NO:13. In various embodiments, HA variant comprises up to 10 mutations relative to SEQ ID NO:13 or ORF of SEQ ID NO:13. In various embodiments, HA variant comprises up to 5 mutations relative to SEQ ID NO:13 or ORF of SEQ ID NO:13. In various embodiments, HA variant comprises up to 4, 3, 2, or 1 mutation relative to SEQ ID NO:13 or ORF of SEQ ID NO:13.

In various embodiments the nucleic acid sequence of the NA variant of SEQ ID NO:14 or ORF of SEQ ID NO:14 does not comprise the wild-type sequence for encoding the NA protein. For example, it does not include the wild-type NA encoding sequence from Influenza A virus A/California/07/2009. In various embodiments the nucleic acid sequence of the NA variant of SEQ ID NO:14 or ORF of SEQ ID NO:14 comprises up to 20 mutations relative to SEQ ID NO:14 or ORF of SEQ ID NO:14. In various embodiments, NA variant comprises up to 10 mutations relative to SEQ ID NO:14 or ORF of SEQ ID NO:14. In various embodiments, NA variant comprises up to 5 mutations relative to SEQ ID NO:14 or ORF of SEQ ID NO:14. In various embodiments, NA variant comprises up to 4, 3, 2, or 1 mutation relative to SEQ ID NO: 14 or ORF of SEQ ID NO:14.

In various embodiments, the M, PB2, PB1, PA, NS or NP protein are each encoded by a nucleic acid having SEQ ID NOs: 3, 4, 5, 6, 7, and 8, respectively, or a variant of M, PB2, PB1, PA, NS or NP variant of SEQ ID NOs: 3, 4, 5, 6, 7, and 8, respectively.

In various embodiments the M, PB2, PB1, PA, NS or NP variant of SEQ ID NOs: 3, 4, 5, 6, 7, and 8, respectively, each comprises up to 20 mutations relative to SEQ ID NOs: 3, 4, 5, 6, 7, and 8, respectively. In various embodiments, the M, PB2, PB1, PA, NS or NP variant comprises up to 10 mutations relative to SEQ ID NOs: 3, 4, 5, 6, 7, and 8, respectively. In various embodiments, the M, PB2, PB1, PA, NS or NP variant comprises up to 5 mutations relative to SEQ ID NOs: 3, 4, 5, 6, 7, and 8, respectively. In various embodiments, the M, PB2, PB1, PA, NS or NP variant comprises up to 4, 3, 2, or 1 mutation relative to SEQ ID NOs: 3, 4, 5, 6, 7, and 8, respectively.

In various embodiments the M, PB2, PB1, PA, NS or NP variant of SEQ ID NOs: 3, 4, 5, 6, 7, and 8, does not comprise wild-type sequence for encoding M, PB2, PB1, PA NS or NP proteins, respectively.

Various embodiments of the present invention provide for a method of treating a malignant tumor, comprising: administering prime dose of a deoptimized influenza virus to a subject in need thereof, wherein an HA protein of the deoptimized influenza virus is encoded by a nucleic acid having at least 99% sequence identity to SEQ ID NO: 1, SEQ ID NO:9, or SEQ ID NO:10, wherein the HA gene does not comprise a nucleic acid having SEQ ID NO:11; an NA protein of the deoptimized influenza virus is encoded by a nucleic acid having at least 99% sequence identity to SEQ ID NO:2, wherein the NA gene does not comprise a nucleic acid having SEQ ID NO:12; an M, PB2, PB1, PA, NS and NP protein of the deoptimized influenza virus are each encoded by a nucleic acid having at least 99% sequence identity to SEQ ID NOs: 3, 4, 5, 6, 7, and 8, respectively; and administering one or more boost doses of the deoptimized influenza virus. In various embodiments, the HA gene does not comprise the ORF of SEQ ID NO:11. In various embodiments, the NA gene does not comprise the ORF of SEQ ID NO:12. In various embodiments, the deoptimized virus' genome as a whole does not comprise a wild-type influenza virus genome as a whole.

Various embodiments of the present invention provide for a method of treating a malignant tumor, comprising: administering prime dose of a deoptimized influenza virus to a subject in need thereof, wherein an HA protein encoded by a nucleic acid having at least 99% sequence identity to SEQ ID NO:13 or ORF of SEQ ID NO:13, wherein the HA gene does not comprise a nucleic acid having SEQ ID NO: 11; an NA protein encoded by a nucleic acid having at least 99% sequence identity to SEQ ID NO:14 or ORF of SEQ ID NO:14, wherein the NA gene does not comprise a nucleic acid having SEQ ID NO:12; an M, PB2, PB1, PA, NS and NP protein are each encoded by a nucleic acid having at least 99% sequence identity to SEQ ID NOs: 3, 4, 5, 6, 7, and 8, respectively; and administering one or more boost doses of the deoptimized influenza virus. In various embodiments, the HA gene does not comprise the ORF of SEQ ID NO:11. In various embodiments, the NA gene does not comprise the ORF of SEQ ID NO:12. In various embodiments, the deoptimized virus' genome as a whole does not comprise a wild-type influenza virus genome as a whole.

In various embodiments, mutation comprises a synonymous substitution. That is, the amino acid remains the same. In various embodiments the one or more mutations comprise a nonsynonymous substitution. That is, the mutation results in a change in the amino acid. In various embodiments wherein more than one mutation occurs, the mutations can be all synonymous substitutions, all nonsynonymous substitutions, or both.

In various embodiments, the prime dose is administered subcutaneously, intramuscularly, intradermally, intranasally, or intravenously. In various embodiments, the prime dose is administered intratumorally.

In various embodiments, a composition comprising about 10⁵-10⁹ PFU of the deoptimized influenza virus is administered as the prime dose. In various embodiments, a composition comprising about 10⁵-10⁹ PFU of the deoptimized influenza virus is administered intratumorally as the prime dose. In various embodiments, an amount of about 10⁵-10⁹ PFU is administered subcutaneously, intramuscularly, intradermally, intranasally, or intravenously as the prime dose.

In various embodiments, a composition comprising about 10⁶-10⁸ PFU of the deoptimized influenza virus is administered as the prime dose. In various embodiments, a composition comprising about 10⁶-10¹ PFU of the deoptimized influenza virus is administered intratumorally as the prime dose. In various embodiments, an amount of about 10⁶-10¹ PFU is administered subcutaneously, intramuscularly, intradermally, intranasally, or intravenously as the prime dose.

In various embodiments, a composition comprising about 10⁷-10¹ PFU of the deoptimized influenza virus is administered as the prime dose. In various embodiments, a composition comprising about 10⁷-10¹ PFU of the deoptimized influenza virus is administered intratumorally as the prime dose. In various embodiments, an amount of about 10⁷-10¹ PFU is administered subcutaneously, intramuscularly, intradermally, intranasally, or intravenously as the prime dose.

In various embodiments, a composition comprising about 10⁶ PFU of the deoptimized influenza virus is administered as the prime dose. In various embodiments, a composition comprising about 10⁷ PFU of the deoptimized influenza virus is administered intratumorally as the prime dose. In various embodiments, an amount of about 10′ PFU is administered subcutaneously, intramuscularly, intradermally, intranasally, or intravenously as the prime dose. In various embodiments, an amount of about 5×10⁸ PFU is administered subcutaneously, intramuscularly, intradermally, intranasally, or intravenously as the prime dose.

In various embodiments, the boost dose is administered subcutaneously, intramuscularly, intradermally, intranasally, or intravenously. In various embodiments, the boost dose is administered intratumorally.

In various embodiments, a composition comprising about 10⁵-10⁹ PFU of the deoptimized influenza virus is administered as the boost dose. In various embodiments, a composition comprising about 10⁵-10⁹ PFU of the deoptimized influenza virus is administered intratumorally as the boost dose. In various embodiments, an amount of about 10⁵-10⁹ PFU is administered subcutaneously, intramuscularly, intradermally, intranasally, or intravenously as the boost dose.

In various embodiments, a composition comprising about 10⁶-10⁸ PFU of the deoptimized influenza virus is administered as the boost dose. In various embodiments, a composition comprising about 10⁶-10⁸ PFU of the deoptimized influenza virus is administered intratumorally as the boost dose. In various embodiments, an amount of about 10⁶-10⁸ PFU is administered subcutaneously, intramuscularly, intradermally, intranasally, or intravenously as the boost dose.

In various embodiments, a composition comprising about 10⁷-10⁸ PFU of the deoptimized influenza virus is administered as the boost dose. In various embodiments, a composition comprising about 10⁷-10⁸ PFU of the deoptimized influenza virus is administered intratumorally as the boost dose. In various embodiments, an amount of about 10⁷-10⁸ PFU is administered subcutaneously, intramuscularly, intradermally, intranasally, or intravenously as the boost dose.

In various embodiments, a composition comprising about 10⁶ PFU of the deoptimized influenza virus is administered as the boost dose. In various embodiments, a composition comprising about 10⁷ PFU of the deoptimized influenza virus is administered intratumorally as the boost dose. In various embodiments, an amount of about 10⁸ PFU is administered subcutaneously, intramuscularly, intradermally, intranasally, or intravenously as the boost dose. In various embodiments, an amount of about 5×10⁸ PFU is administered subcutaneously, intramuscularly, intradermally, intranasally, or intravenously as the boost dose.

In various embodiments, a first of the one or more boost dose is administered about 2 weeks after one prime dose, or if more than one prime dose then about 2 weeks after the last prime dose.

In various embodiments, the one or more boost doses of the deoptimized influenza virus are administered about every after the prime dose. In various embodiments, the one or more boost doses of the deoptimized influenza virus are administered every 2-3 days after the prime dose for up to 4 weeks.

In various embodiments, the one or more boost doses of the deoptimized influenza virus are administered every 1-6 weeks after the prime dose for 2-6 total doses. In various embodiments, the one or more boost doses of the deoptimized influenza virus are administered every 2-5 weeks after the prime dose for 2-6 total doses. In various embodiments, the one or more boost doses of the deoptimized influenza virus are administered every 2-4 weeks after the prime dose for 3-5 total doses.

In various embodiments, the subject has cancer.

In various embodiments, the prime dose is administered when the subject does not have cancer. In various embodiments, the subject is at a higher risk of developing cancer. In various embodiments, the one or more boost dose is administered about every 1, 2, 3, 4, 5, 6, 7, 8, 9 or 10 years after the prime dose when the subject does not have cancer. In various embodiments, the one or more boost dose is administered after the subject is diagnosed with cancer.

In various embodiments, the method further comprises administering a PD-1 inhibitor or a PD-L1 inhibitor. In various embodiments, the PD-1 inhibitor is an anti-PD1 antibody. In various embodiments, the anti-PD1 antibody is selected from the group consisting of pembrolizumab, nivolumab, pidilizumab, AMP-224, AMP-514, spartalizumab, cemiplimab, AGEN2034/balstilimab, AK105, BCD-100, BI 754091, JS001, LZM009, MGA012, Sym021, TSR-042/dostarlimab, MGD013, AK104, XmAb207I7, tislelizumab, and combinations thereof. In various embodiments, the PD-1 inhibitor is selected from the group consisting of PF-06801591, anti-PD1 antibody expressing pluripotent killer T lymphocytes (PIK-PD-1), autologous anti-EGFRvIII 4SCAR-IgT cells, and combinations thereof. In various embodiments, the PD-L1 inhibitor is an anti-PD-L1 antibody. In various embodiments, the anti-PD-L1 antibody is selected from the group consisting of BGB-A333, CK-301, FAZ053, KN035, MDX-1105, MSB2311, SHR-1316, atezolizumab, avelumab, durvalumab, BMS-936559, CK-301, and combinations thereof. In various embodiments, the anti-PD-L1 inhibitor is M7824/bintrafusp alpha.

In various embodiments, the method further comprises administering a chemotherapeutic agent. For example, taxanes (paclitaxel, nab-paclitaxel, docetaxel), platinum based therapies (cisplatin), gemcitabine, doxorubicin, or cyclophosphamide.

Additional examples of chemotherapeutic agent include but are not limited to chemotherapeutic agents include cytotoxic agents (e.g., 5-fluorouracil, cisplatin, carboplatin, methotrexate, daunorubicin, doxorubicin (Adriamycin®), vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic akylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

In various embodiments, the method further comprises administering a cancer immunotherapy. For example, CTLA-4 blockade, LAG-3 blockade, and agonist of the CD226/TIGIT axis.

In various embodiments, the method further comprises administration of an additional therapeutic agent. Examples of therapeutic agents that may be used in accordance with various embodiments of the present invention include: anti-cancer drugs (including chemotherapeutic agents and antiproliferative agents), therapeutic viral particles, antimicrobials (e.g., antibiotics, antifingals, antivirals), cytokines and therapeutic proteins, immunotoxins, immunosuppressants, and gene therapeutics (e.g., adenoviral vectors, adeno-associated viral vectors, retroviral vectors, herpes simplex viral vectors, pox virus vectors).

Examples of antiproliferative agents include alkylating agents, antimetabolites, enzymes, biological response modifiers, hormones and antagonists, androgen inhibitors (e.g., flutamide and leuprolide acetate), antiestrogens (e.g., tamoxifen citrate and analogs thereof, toremifene, droloxifene and roloxifene), Additional examples of antiproliferative agents include, but are not limited to levamisole, gallium nitrate, granisetron, sargramostim strontium-89 chloride, filgrastim, pilocarpine, dexrazoxane, and ondansetron.

In various embodiments, treating the malignant tumor decreases the likelihood of recurrence of the malignant tumor.

In various embodiments, treating the malignant tumor decreases the likelihood of having a second cancer that is different from the malignant tumor.

In various embodiments, if the subject develops a second cancer that is different from the malignant tumor, the treatment of the malignant tumor results in slowing the growth of the second cancer.

In various embodiments, after remission of the malignant tumor, if the subject develops a second cancer that is different from the malignant tumor, the treatment of the malignant tumor results in slowing the growth of the second cancer.

In various embodiments, treating the malignant tumor stimulates an inflammatory immune response in the tumor. In various embodiments, treating the malignant tumor recruits pro-inflammatory cells to the tumor. In various embodiments, treating the malignant tumor stimulates an anti-tumor immune response.

In various embodiments, treating the malignant tumor reduced the tumor size.

In various embodiments, the malignant tumor is breast cancer (including triple negative breast cancer), glioblastoma, adenocarcinoma, melanoma, lung carcinoma, neuroblastoma, bladder cancer, colon cancer, prostate cancer, or liver cancer.

In various embodiments, the malignant tumor is a sialyltransferase expressing or overexpressing tumor. In various embodiments, the sialyltransferase is ST6Gal 1. In various embodiments, the sialyltransferase is ST6Gal2. In various embodiments, the sialyltransferase is ST3Gal1, ST3Gal2, ST3Gal4, ST3Gal6, or combinations thereof.

In various embodiments, the malignant tumor is testicular germ cell tumors (TGCT), diffuse large B cell lymphoma (DLBC), pancreatic adenocarcinoma (PAAD) and ovarian serous cystadenocarcinoma (OV), skin cutaneous melanoma (SKCM), tumors of the gastrointestinal tract (stomach (STAD), rectal (READ), colon (COAD), and esophageal (ESCA) carcinomas), lower grade glioma (LGG) and glioblastoma (GBM), thymoma (THYM), or hepatocellular carcinoma (LH-1Q.

In various embodiments, the malignant tumor is pancreatic adenocarcinomoa (PAAD) or melanoma (SKCM).

Sequences

| | | SEQ |
|---|---|---|
| | | ID |
| Description | Sequence | NO: |
| HA P4 | AGCAAAAGCAGGGGAAAATAAAAGCAACAAAAATGAAGGCAATAC | 1 |
| | TAGTAGTTCTGCTATATACATTTGCAACCGCAAATGCAGACACAT | |
| | TATGTATAGGTTATCATGCGAACAATTCAACAGACACTGTAGACA | |
| | CAGTACTAGAAAAGAATGTAACAGTAACACACTCTGTTAACCTAT | |
| | TGGAGGATAAGCATAACGGTAAGCTATGCAAACTGAGAGGCGTAG | |
| | CACCATTGCATCTAGGTAAGTGTAATATAGCCGGATGGATTCTAG | |
| | GGAATCCCGAATGCGAATCACTATCAACCGCTAGCTCATGGTCAT | |
| | ACATAGTCGAAACACCATCAAGCGATAACGGTACATGTTATCCCG | |
| | GAGACTTTATCGATTACGAAGAGCTTAGAGAGCAATTGTCTAGCG | |
| | TAAGCTCATTCGAAAGATTCGAAATTTTTCCGAAAACTAGCTCAT | |
| | GGCCTAATCACGATAGTAATAAAGGCGTAACTGCCGCATGCCCAC | |
| | ACGCCGGAGCTAAATCATTCTATAAGAATCTGATTTGGTTAGTGA | |
| | AAAAAGGGAATTCATATCCGAAACTATCTAAATCATACATTAACG | |
| | ATAAGGGTAAGGAGGTACTAGTGTTGTGGGGGATACACCATCCAT | |
| | CAACTAGCGCCGATCAGCAATCATTGTATCAGAACGCAGACGCAT | |
| | ACGTATTCGTAGGGTCTAGTAGATACTCTAAAAAATTTAAACCCG | |
| | AAATCGCAATTAGACCGAAAGTGAGAGACCAAGAGGGTAGAATGA | |
| | ATTACTATTGGACACTAGTCGAACCAGGCGATAAGATTACATTCG | |
| | AAGCGACAGGGAATCTAGTCGTACCGAGATACGCATTCGCAATGGA | |
| | GAGAAACGCCGGATCCGGAATTATTATTAGCGATACACCCGTACA | |
| | CGATTGCAATACTACATGTCAGACACCAAAAGGCGCAATTAATAC | |
| | TAGTCTGCCATTTCAGAATATACACCCAATTACAATCGGTAAGTG | |
| | TCCAAAATACGTTAAGTCAACTAAGTTGAGACTCGCAACAGGGTT | |
| | GAGAAATATACCGTCAATTCAATCTAGGGGGTTGTTCGGAGCAAT | |
| | CGCAGGGTTTATCGAAGGGGGGTGGACAGGTATGGTTGACGGATG | |
| | GTACGGATACCATCATCAAAACGAACAGGGATCCGGATACGCAGC | |
| | CGATCTGAAAAGTACACAGAACGCTATAGACGAAATTACGAATAA | |
| | AGTGAATAGCGTAATCGAAAAAATGAATACGCAATTTACAGCCGT | |
| | AGGTAAGGAGTTTAATCATCTCGAAAAAAGGATTGAGAATCTGAA | |
| | TAAAAAAGTCGACGACGGATTCTTAGACATTTGGACTTATAACGC | |
| | CGAACTGTTAGTGTTACTCGAAAACGAAAGAACACTAGACTATCA | |
| | CGATTCAAACGTTAAGAATCTATACGAAAAAGTGAGATCGCAATT | |
| | GAAAAATAACGCTAAAGAGATAGGGAATGGGTGTTTCGAATTCTA | |
| | TCATAAATGCGATAATACATGTATGGAATCCGTTAAAAACGGAAC | |
| | ATACGATTACCCTAAGTATAGCGAAGAGGCTAAACTGAATAGGGA | |
| | AGAGATAGACGGAGTGAAACTCGAATCAACTAGGATITATCAGAT | |
| | ACTCGCAATTTATAGTACGGTTGCCAGTTCATTGGTACTGGTAGT | |
| | CTCCCTGGGGGCAATCAGTTTCTGGATGTGCTCTAATGGGTCTCT | |
| | ACAGTGTAGAATATGTATTTAACATTAGGATITCAGAAGCATGAG | |
| | AAAAACACCCTTGTTTCTACT | |
| NA P4 | AGCAAAAGCAGGGGTTTAAAATGAATCCAAACCAAAAGATAATAA | 2 |
| | CCATTGGTTCGGTCTGTATGACAATTGGAATGGCTAACTTAATAT | |
| | TACAAATTGGAAACATAATCTCAATATGGATTAGCCACTCAATCC | |
| | AATTGGGGAATCAGAATCAAATCGAAACATGCAATCAATCCGTAA | |
| | TTACATACGAGAATAATACTTGGGTGAATCAGACATACGTTAACA | |
| | TATCGAATACTAATTTCGCTGCCGGACAATCCGTCGTGAGTGTGA | |
| | AACTAGCCGGTAATAGTAGTCTATGTCCCGTTAGCGGATGGGCTA | |
| | TATACTCTAAAGACAATAGCGTTAGAATCGGATCTAAAGGCGACG | |
| | TATTCGTTATACGCGAACCATTCATAAGTTGTAGTCCATTAGAGT | |
| | GTAGGACTTTTTTTCTGACACAGGGCGCACTATTGAACGATAAGC | |
| | ATTCTAACGGTACAATCAAAGATAGGTCACCATATAGAACACTAA | |
| | TGTCATGTCCGATAGGCGAAGTGCCTAGTCCATACAATAGTAGAT | |
| | TCGAATCCGTCGCTTGGTCCGCTAGCGCATGCCATGACGGTATTA | |
| | ATTGGTTGACAATCGGGATTAGCGGACCCGATAACGGCGCAGTCG | |
| | CCGTACTTAAGTATAACGGTATAATTACCGATACTATTAAGAGTT | |
| | GGCGAAATAATATATTGCGAACACAGGAATCCGAATGCGCATGCG | |
| | TTAACGGATCATGTITTACCGTTATGACTGACGGACCATCTAACG | |
| | GGCAAGCGTCATATAAGATTTTTAGAATCGAAAAAGGTAAGATAG | |
| | TGAAATCCGTCGAAATGAACGCTCCTAATTATCATTACGAAGAGT | |
| | GCTCATGTTATCCCGATTCTAGCGAAATTACATGCGTATGTAGAG | |
| | ACAATTGGCACGGATCTAATAGACCTTGGGTGTCATTCAATCAGA | |
| | ATCTAGAGTATCAAATCGGGTATATATGCTCAGGGATATTCGGAG | |
| | ACAATCCTAGACCTAACGATAAGACAGGGTCATGCGGACCAGTGA | |
| | GTTCTAACGGCGCTAACGGCGTTAAGGGGTTTAGTTTCAAATACG | |
| | GTAACGGCGTATGGATAGGGAGAACTAAGTCAATCTCTAGTAGAA | |
| | ACGGATTCGAAATGATATGGGACCCTAACGGATGGACCGGAACCG | |
| | ATAATAATTTTTCGATTAAACAGGATATCGTAGGGATTAACGAAT | |
| | GGTCAGGGTATAGCGGATCATTCGTACAGCATCCAGAGTTAACCG | |
| | GACTCGATTGCATACGACCATGTTTTTGGGTCGAACTGATTAGGG | |
| | GGAGACCGAAAGAGAATACTATATGGACTAGCGGGAGCAGCATAT | |
| | CCTTTTGTGGTGTAAACAGTGACACTGTGGGTTGGTCTTGGCCAG | |
| | ACGGTGCTGAGTTGCCATTTACCATTGACAAGTAATTTGTTCAAA | |
| | AAACTCCTTGTTTCTACT | |
| MP4 | AGCAAAAGCAGGTAGATATTTAAAGATGAGTCTTCTAACCGAGGT | 3 |
| | CGAAACGTACGTTCTTTCTATCATCCCGTCAGGCCCCCTCAAAGC | |
| | CGAGATCGCGCAGAGACTGGAAAGTGTCTTTGCAGGAAAGAACAC | |
| | AGATCTTGAGGCTCTCATGGAATGGCTAAAGACAAGACCAATCTT | |
| | GTCACCTCTGACTAAGGGAATTTTAGGATTTGTGTTCACGCTCAC | |
| | CGTGCCCAGTGAGCGAGGACTGCAGCGTAGACGCTTTGTCCAAAA | |
| | TGCCCTAAATGGGAATGGGGACCCGAACAACATGGATAGAGCAGT | |
| | TAAACTATACAAGAAGCTCAAAAGAGAAATAACGTTCCATGGGGC | |
| | CAAGGAGGTGTCACTAAGCTATTCAACTGGTGCACTTGCCAGTTG | |
| | CATGGGCCTCATATACAACAGGATGGGAACAGTGACCACAGAAGC | |
| | TGCTTTTGGTCTAGTGTGTGCCACTTGTGAACAGATTGCTGATTC | |
| | ACAGCATCGGTCTCACAGACAGATGGCTACTACCACCAATCCACT | |
| | AATCAGGCATGAAAACAGAATGGTGCTGGCTAGCACTACGGCAAA | |
| | GGCTATGGAACAGATGGCTGGATCGAGTGAACAGGCAGCGGAGGC | |
| | CATGGAGGTTGCTAATCAGACTAGGCAGATGGTACATGCAATGAG | |
| | AACTATTGGGACTCATCCTAGCTCCAGTGCTGGTCTGAAAGATGA | |
| | CCTTCTTGAAAATTTGCAGGCCTACCAGAAGCGAATGGGAGTGCA | |
| | GATGCAGCGATTCAAGTGATCCTCTCGTCATTGCAGCAAATATCA | |
| | TTGGGATCTTGCACCTGATATTGTGGATTACTGATCGTCTTTTTT | |
| | TCAAATGTATTTATCGTCGCTTTAAATACGGTTTGAAAAGAGGGC | |
| | CTTCTACGGAAGGAGTGCCTGAGTCCATGAGGGAAGAATATCAAC | |
| | AGGAACAGCAGAGTGCTGTGGATGTTGACGATGGTCATTTTGTCA | |
| | ACATAGAGCTAGAGTAAAAAACTACCTTGTTTCTACT | |
| PB2 P4 | AGCGAAAGCAGGTCAAATATATTCAATATGGAGAGAATAAAAGAA | 4 |
| | CTGAGAGATCTAATGTCGCAGTCCCGCACTCGCGAGATACTCACT | |
| | AAGACCACTGTGGACCATATGGCCATAATCAAAAAGTACACATCA | |
| | GGAAGGCAAGAGAAGAACCCCGCACTCAGAATGAAGTGGATGATG | |
| | GCAATGAGATACCCAATTACAGCAGACAAGAGAATAATGGACATG | |
| | ATTCCAGAGAGGAATGAACAAGGACAAACCCTCTGGAGCAAAACA | |
| | AACGATGCTGGATCAGACCGAGTGATGGTATCACCTCTGGCCGTA | |
| | ACATGGTGGAATAGGAATGGCCCAACAACAAGTACAGTTCATTAC | |
| | CCTAAGGTATATAAAACTTATTTCGAAAAGGTCGAAAGGTTGAAA | |
| | CATGGTACCTTCGGCCCTGTCCACTTCAGAAATCAAGTTAAAATA | |
| | AGGAGGAGAGTTGATACAAACCCTGGCCATGCAGATCTCAGTGCC | |
| | AAGGAGGCACAGGATGTGATTATGGAAGTTGTTTTCCCAAATGAA | |
| | GTGGGGGCAAGAATACTGACATCAGAGTCACAGCTGGCAATAACA | |
| | AAAGAGAAGAAAGAAGAGCTCCAGGATTGTAAAATTGCTCCCTTG | |
| | ATGGTGGCGTACATGCTAGAAAGAGAATTGGTCCGTAAAACAAGG | |
| | TTTCTCCCAGTAGCCGGCGGAACAGGCAGTGTTTATATTGAAGTG | |
| | TTGCACTTAACCCAAGGGACGTGCTGGGAGCAGATGTACACTCCA | |
| | GGAGGAGAAGTGAGAAATGATGATGTTGACCAAAGTTTGATTATC | |
| | GCTGCTAGAAACATAGTAAGAAGAGCAGCAGTGTCAGCAGACCCA | |
| | TTAGCATCTCTCTTGGAAATGTGCCACAGCACACAGATTGGAGGA | |
| | GTAAGGATGGTGGACATCCTTAGACAGAATCCAACTGAGGAACAA | |
| | GCCGTAGACATATGCAAGGCAGCAATAGGGTTGAGGATTAGCTCA | |
| | TCTTTCAGTTTTGGTGGGTTCACTTTCAAAAGGACAAGCGGATCA | |
| | TCAGTCAAGAAAGAAGAAGAAGTGCTAACGGGCAACCTCCAAACA | |
| | CTGAAAATAAGAGTACATGAAGGGTATGAAGAATTCACAATGGTT | |
| | GGGAGAAGAGCAACAGCTATTCTCAGAAAGGCAACCAGGAGATTG | |
| | ATCCAGTTGATAGTAAGCGGGAGAGACGAGCAGTCAATTGCTGAG | |
| | GCAATAATTGTGGCCATGGTATTCTCACAGGAGGATTGCATGATC | |
| | AAGGCAGTTAGGGGCGATCTGAACTTTGTCAATAGGGCAAACCAG | |
| | CGACTGAACCCCATGCACCAACTCTTGAGGCATTTCCAAAAAGAT | |
| | GCAAAAGTGCTTTTCCAGAACTGGGGAATTGAATCCATCGACAAT | |
| | GTGATGGGAATGATCGGAATACTGCCCGACATGACCCCAAGCACG | |
| | GAGATGTCGCTGAGAGGGATAAGAGTCAGCAAAATGGGAGTAGAT | |
| | GAATACTCCAGCACGGAGAGAGTGGTAGTGAGTATTGACCGATTT | |
| | TTAAGGGTTAGAGATCAAAGAGGGAACGTACTATTGTCTCCCGAA | |
| | GAAGTCAGTGAAACGCAAGGAACTGAGAAGTTGACAATAACTTAT | |
| | TCGTCATCAATGATGTGGGAGATCAATGGCCCTGAGTCAGTGCTA | |
| | GTCAACACTTATCAATGGATAATCAGGAACTGGGAAATTGTGAAA | |
| | ATTCAATGGTCACAAGATCCCACAATGTTATACAACAAAATGGAA | |
| | TTTGAACCATTTCAGTCTCTTGTCCCTAAGGCAACCAGAAGCCGG | |
| | TACAGTGGATTCGTAAGGACACTGTTCCAGCAAATGCGGGATGTG | |
| | CTTGGGACATTTGACACTGTCCAAATAATAAAACTTCTCCCCTTT | |
| | GCTGCTGCCCCACCAGAACAGAGTAGGATGCAATTTTCCTCATTG | |
| | ACTGTGAATGTGAGAGGATCAGGGTTGAGGATACTGGTAAGAGGC | |
| | AATTCTCCAGTATTCAATTACAACAAGGCAACCAAACGACTTACA | |
| | GTTCTTGGAAAGGATGCAGGTGCATTGACTGAAGATCCAGATGAA | |
| | GGCACATCTGGGGTGGAGTCTGCTGTCCTGAGAGGATTTCTCATT | |
| | TTGGGCAAAGAAGACAAGAGATATGGCCCAGCATTAAGCATCAAT | |
| | GAACTGAGCAATCTTGCAAAAGGAGAGAAGGCTAATGTGCTAATT | |
| | GGGCAAGGGGACGTAGTGTTGGTAATGAAACGAAAACGGGACTCT | |
| | AGCATACTTACTGACAGCCAGACAGCGACCAAAAGAATTCGGATG | |
| | GCCATCAATTAGTGTCGAATTGTTTAAAAACGACCTTGTTTCTAC | |
| | T | |
| PB1 P4 | AGCGAAAGCAGGCAAACCATTTGAATGGATGTCAATCCGACTCTA | 5 |
| | CTTTTCCTAAAAATTCCAGCGCAAAATGCCATAAGCACCACATTC | |
| | CCTTATACTGGAGATCCTCCATACAGCCATGGAACAGGAACAGGA | |
| | TACACCATGGACACAGTAAACAGAACACACCAATACTCAGAAAAG | |
| | GGAAAGTGGACGACAAACACAGAGACTGGTGCACCCCAGCTCAAC | |
| | CCGATTGATGGACCACTACCTGAGGATAATGAACCAAGTGGGTAT | |
| | GCACAAACAGACTGTGTTCTAGAGGCTATGGCTTTCCTTGAAGAA | |
| | TCCCACCCAGGAATATTTGAGAATTCATGCCTTGAAACAATGGAA | |
| | GTIGTTCAACAAACAAGGGTAGATAAACTAACTCAAGGTCGCCAG | |
| | ACTTATGATTGGACATTAAACAGAAATCAACCGGCAGCAACTGCA | |
| | TTGGCCAACACCATAGAAGTCTTTAGATCGAATGGCCTAACAGCT | |
| | AATGAGTCAGGAAGGCTAATAGATTTCTTAAAGGATGTAATGGAA | |
| | TCAATGAACAAAGAGGAAATAGAGATAACAACCCACTTTCAAAGA | |
| | AAAAGGAGAGTAAGAGACAACATGACCAAGAAGATGGTCACGCAA | |
| | AGAACAATAGGGAAGAAAAAACAAAGACTGAATAAGAGAGGCTAT | |
| | CTAATAAGAGCACTGACATTAAATACGATGACCAAAGATGCAGAG | |
| | AGAGGCAAGTTAAAAAGAAGGGCTATCGCAACACCTGGGATGCAG | |
| | ATTAGAGGTTTCGTATACTTTGTTGAAACTTTAGCTAGGAGCATT | |
| | TGCGAAAAGCTTGAACAGTCTGGGCTCCCAGTAGGGGGCAATGAA | |
| | AAGAAGGCCAAACTGGCAAATGTTGTGAGAAAGATGATGACTAAT | |
| | TCACAAGACACAGAGATTTCTTTCACAATCACTGGGGACAACACT | |
| | AAGTGGAATGAAAATCAAAATCCTCGAATGTTCCTGGCGATGATT | |
| | ACATATATCACCAGAAATCAACCCGAGTGGTTCAGAAACATCCTG | |
| | AGCATGGCACCCATAATGTTCTCAAACAAAATGGCAAGACTAGGG | |
| | AAAGGGTACATGTTCGAGAGTAAAAGAATGAAGATTCGAACACAA | |
| | ATACCAGCAGAAATGCTAGCAAGCATTGACCTGAAGTACTTCAAT | |
| | GAATCAACAAAGAAGAAAATTGAGAAAATAAGGCCTCTTCTAATA | |
| | GATGGCACAGCATCACTGAGTCCTGGGATGATGATGGGCATGTTC | |
| | AACATGCTAAGTACGGTCTTGGGAGTCTCGATACTGAATCTTGGA | |
| | CAAAAGAAATACACCAAGACAATATACTGGTGGGATGGGCTCCAA | |
| | TCATCCGACGATTTTGCTCTCATAGTGAATGCACCAAACCATGAG | |
| | GGAATACAAGCAGGAGTGGACAGATTCTACAGGACCTGCAAGTTA | |
| | GTGGGAATCAACATGAGCAAAAAGAAGTCCTATATAAATAAGACA | |
| | GGGACATTTGAATTCACAAGCTTTTTTTATCGCTATGGATTTGTG | |
| | GCTAATTTTAGCATGGAGCTACCCAGCTTTGGAGTGTCTGGAGTA | |
| | AATGAATCAGCTGACATGAGTATTGGAGTAACAGTGATAAAGAAC | |
| | AACATGATAAACAATGACCTTGGACCTGCAACGGCCCAGATGGCT | |
| | CTTCAATTGTTCATCAAAGACTACAGATACACATATAGGTGCCAT | |
| | AGGGGAGACACACAAATTCAGACGAGAAGATCATTTGAGTTAAAG | |
| | AAGCTGTGGGATCAAACCCAATCAAAAGTAGGGCTATTAGTATCA | |
| | GATGGAGGACCAAACTTATACAATATACGGAATCTTCACATTCCT | |
| | GAAGTCTGCTTAAAATGGGAGCTAATGGATGATGATTATCGGGGA | |
| | AGACTTTGTAATCCCCTGAATCCCTTTGTCAGTCATAAAGAGATT | |
| | GATTCTGTAAACAATGCTGTGGTAATGCCAGCCCATGGTCCAGCC | |
| | AAAAGCATGGAATATGATGCCGTTGCAACTACACATTCCTGGATT | |
| | CCCAAGAGGAATCGTTCTATTCTCAACACAAGCCAAAGGGGAATT | |
| | CTTGAGGATGAACAGATGTACCAGAAGTGCTGCAATCTATTCGAG | |
| | AAATTTTTCCCTAGCAGTTCATATAGGAGACCGGTTGGAATTTCT | |
| | AGCATGGTGGAGGCCATGGTGTCTAGGGCCCGGATTGATGCCAGG | |
| | GTCGACTTCGAGTCTGGACGGATCAAGAAAGAAGAGTTCTCTGAG | |
| | ATCATGAAGATCTGTTCCACCATTGAAGAACTCAGACGGCAAAAA | |
| | TAATGAATTTAACTTGTCCTTCATGAAAAAATGCCTTGTTTCTAC | |
| | T | |
| PA P4 | AGCGAAAGCAGGTACTGATCCAAAATGGAAGACTTTGTGCGACAA | 6 |
| | TGCTTCAATCCAATGATCGTCGAGCTTGCGGAAAAGGCAATGAAA | |
| | GAATATGGGGAAGATCCGAAAATCGAAACTAACAAGTTTGCTGCA | |
| | ATATGCACACATTTGGAAGTTTGTTTCATGTATTCGGATTTCCAT | |
| | TTCATCGACGAACGGGGTGAATCAATAATTGTAGAATCTGGTGAC | |
| | CCGAATGCACTATTGAAGCACCGATTTGAGATAATTGAAGGAAGA | |
| | GACCGAATCATGGCCTGGACAGTGGTGAACAGTATATGTAACACA | |
| | ACAGGGGTAGAGAAGCCTAAATTTCTTCCTGATTTGTATGATTAC | |
| | AAAGAGAACCGGTTCATTGAAATTGGAGTAACACGGAGGGAAGTC | |
| | CACATATATTACCTAGAGAAAGCCAACAAAATAAAATCTGAGAAG | |
| | ACACACATTCACATCTTTTCATTCACTGGAGAGGAGATGGCCACC | |
| | AAAGCGGACTACACCCTTGACGAAGAGAGCAGGGCAAGAATCAAA | |
| | ACTAGGCTTTTCACTATAAGACAAGAAATGGCCAGTAGGAGTCTA | |
| | TGGGATTCCTTTCGTCAGTCCGAAAGAGGCGAAGAGACAATTGAA | |
| | GAAAAATTTGAGATTACAGGAACTATGCGCAAGCTTGCCGACCAA | |
| | AGTCTCCCACCGAACTTCCCCAGCCTTGAAAACTTTAGAGCCTAT | |
| | GTAGATGGATTCGAGCCGAACGGCTGCATTGAGGGCAAGCTTTCC | |
| | CAAATGTCAAAAGAAGTGAACGCCAAAATTGAACCATTCTTGAGG | |
| | ACGACACCACGCCCCCTCAGATTGCCTGATGGGCCTCTTTGCCAT | |
| | CAGCGGTCAAAGTTCCTGCTGATGGATGCTCTGAAATTAAGTATT | |
| | GAAGACCCGAGTCACGAGGGGGAGGGAATACCACTATATGATGCA | |
| | ATCAAATGCATGAAGACATTCTTTGGCTGGAAAGAGCCTAACATA | |
| | GTCAAACCACATGAGAAAGGCATAAATCCCAATTACCTCATGGCT | |
| | TGGAAGCAGGTGCTAGCAGAGCTACAGGACATTGAAAATGAAGAG | |
| | AAGATCCCAAGGACAAAGAACATGAAGAGAACAAGCCAATTGAAG | |
| | TGGGCACTCGGTGAAAATATGGCACCAGAAAAAGTAGACTTTGAT | |
| | GACTGCAAAGATGTTGGAGACCTTAAACAGTATGACAGTGATGAG | |
| | CCAGAGCCCAGATCTCTAGCAAGCTGGGTCCAAAATGAATTCAAT | |
| | AAGGCATGTGAATTGACTGATTCAAGCTGGATAGAACTTGATGAA | |
| | ATAGGAGAAGATGTTGCCCCGATTGAACATATCGCAAGCATGAGG | |
| | AGGAACTATTTTACAGCAGAAGTGTCCCACTGCAGGGCTACTGAA | |
| | TACATAATGAAGGGAGTGTACATAAATACGGCCTTGCTCAATGCA | |
| | TCCTGTGCAGCCATGGATGACTTTCAGCTGATCCCAATGATAAGC | |
| | AAATGTAGGACCAAAGAAGGAAGACGGAAAACAAACCTGTATGGG | |
| | TTCATTATAAAAGGAAGGTCTCATTTGAGAAATGATACTGATGTG | |
| | GTGAACTTTGTAAGTATGGAGTTCTCACTCACTGACCCGAGACTG | |
| | GAGCCACACAAATGGGAAAAATACTGTGTTCTTGAAATAGGAGAC | |
| | ATGCTCTTGAGGACTGCGATAGGCCAAGTGTCGAGGCCCATGTTC | |
| | CTATATGTGAGAACCAATGGAACCTCCAAGATCAAGATGAAATGG | |
| | GGCATGGAAATGAGGCGCTGCCTTCTTCAGTCTCTTCAGCAGATT | |
| | GAGAGCATGATTGAGGCCGAGTCTTCTGTCAAAGAGAAAGACATG | |
| | ACCAAGGAATTCTTTGAAAACAAATCGGAAACATGGCCAATCGGA | |
| | GAGTCACCCAGGGGAGTGGAGGAAGGCTCTATTGGGAAAGTGTGC | |
| | AGGACCTTACTGGCAAAATCTGTATTCAACAGTCTATATGCGTCT | |
| | CCACAACTTGAGGGGTTTTCGGCTGAATCTAGAAAATTGCTTCTC | |
| | ATTGTTCAGGCACTTAGGGACAACCTGGAACCTGGAACCTTCGAT | |
| | CTTGGGGGGCTATATGAAGCAATCGAGGAGTGCCTGATTAATGAT | |
| | CCCTGGGTTTTGCTTAATGCATCTTGGTTCAACTCCTTCCTCACA | |
| | CATGCACTGAAGTAGTTGTGGCAATGCTACTATTTGCTATCCATA | |
| | CTGTCCAAAAAAGTACCTTGTTTCTACT | |
| NS P4 | AGCAAAAGCAGGGTGACAAAAACATAATGGACTCCAACACCATGT | 7 |
| | CAAGCTTTCAGGTAGACTGTTTCCTTTGGCATATCCGCAAGCGAT | |
| | TTGCAGACAATGGATTGGGTGATGCCCCATTCCTTGATCGGCTCC | |
| | GCCGAGATCAAAAGTCCTTAAAAGGAAGAGGCAACACCCTTGGCC | |
| | TCGATATCGAAACAGCCACTCTTGTTGGGAAACAAATCGTGGAAT | |
| | GGATCTTGAAAGAGGAATCCAGCGAGACACTTAGAATGACAATTG | |
| | CATCTGTACCTACTTCGCGGTACCTTTCTGACATGACCCTCGAGG | |
| | AAATGTCACGAGACTGGTTCATGCTCATGCCTAGGCAAAAGATAA | |
| | TAGGCCCTCTTTGCGTGCGATTGGACCAGGCGATCATGGAAAAGA | |
| | ACATAGTACTGAAAGCGAACTTCAGTGTAATCTTTAACCGATTAG | |
| | AGACCTTGATACTACTAAGGGCTTTCACTGAGGAGGGAGCAATAG | |
| | TTGGAGAAATTTCACCATTACCTTCTCTTCCAGGACATACTTATG | |
| | AGGATGTCAAAAATGCAGTTGGGGTCCTCATCGGAGGACTTGAAT | |
| | GGAATGGTAACACGGTTCGAGTCTCTGAAAATATACAGAGATTCG | |
| | CTTGGAGAAACTGTGATGAGAATGGGAGACCTTCACTACCTCCAG | |
| | AGCAGAAATGAAAAGTGGCGAGAGCAATTGGGACAGAAATTTGAG | |
| | GAAATAAGGTGGTTAATTGAAGAAATGCGGCACAGATTGAAAGCG | |
| | ACAGAGAATAGTTTCGAACAAATAACATTTATGCAAGCCTTACAA | |
| | CTACTGCTTGAAGTAGAACAAGAGATAAGAGCTTTCTCGTTTCAG | |
| | CTTATTTAATGATAAAAAACACCCTTGTTTCTACT | |
| NP P4 | AGCAAAAGCAGGGTAGATAATCACTCAATGAGTGACATCGAAGCC | 8 |
| | ATGGCGTCTCAAGGCACCAAACGATCATATGAACAAATGGAGACT | |
| | GGTGGGGAGCGCCAGGATGCCACAGAAATCAGAGCATCTGTCGGA | |
| | AGAATGATTGGTGGAATCGGGAGATTCTACATCCAAATGTGCACT | |
| | GAACTCAAACTCAGTGATTATGATGGACGACTAATCCAGAATAGC | |
| | ATAACAATAGAGAGGATGGTGCTTTCTGCTTTTGATGAGAGAAGA | |
| | AATAAATACCTAGAAGAGCATCCCAGTGCTGGGAAGGACCCTAAG | |
| | AAAACAGGAGGACCCATATATAGAAGAGTAGACGGAAAGTGGATG | |
| | AGAGAACTCATCCTTTATGACAAAGAAGAAATAAGGAGAGTTTGG | |
| | CGCCAAGCAAACAATGGCGAAGATGCAACAGCAGGTCTTACTCAT | |
| | ATCATGATTTGGCATTCCAACCTGAATGATGCCACATATCAGAGA | |
| | ACAAGAGCGCTTGTTCGCACCGGAATGGATCCCAGAATGTGCTCT | |
| | CTAATGCAAGGTTCAACACTTCCCAGAAGGTCTGGTGCCGCAGGT | |
| | GCTGCGGTGAAAGGAGTTGGAACAATAGCAATGGAGTTAATCAGA | |
| | ATGATCAAACGTGGAATCAATGACCGAAATTTCTGGAGGGGTGAA | |
| | AATGGACGAAGGACAAGGGTTGCTTATGAAAGAATGTGCAATATC | |
| | CTCAAAGGAAAATTTCAAACAGCTGCCCAGAGGGCAATGATGGAT | |
| | CAAGTAAGAGAAAGTCGAAACCCAGGAAACGCTGAGATTGAAGAC | |
| | CTCATTTTCCTGGCACGGTCAGCACTCATTCTGAGGGGATCAGTT | |
| | GCACATAAATCCTGCCTGCCTGCTTGTGTGTATGGGCTTGCAGTA | |
| | GCAAGTGGGCATGACTTTGAAAGGGAAGGGTACTCACTGGTCGGG | |
| | ATAGACCCATTCAAATTACTCCAAAACAGCCAAGTGGTCAGCCTG | |
| | ATGAGACCAAATGAAAACCCAGCTCACAAGAGTCAATTGGTGTGG | |
| | ATGGCATGCCACTCTGCTGCATTTGAAGATTTAAGAGTATCAAGT | |
| | TTCATAAGAGGAAAGAAAGTGATTCCAAGAGGAAAGCTTTCCACA | |
| | AGAGGGGTCCAGATTGCTTCAAATGAGAATGTGGAAACCATGGAC | |
| | TCCAATACCCTGGAACTGAGAAGCAGATACTGGGCCATAAGGACC | |
| | AGGAGTGGAGGAAATACCAATCAACAAAAGGCATCCGCAGGCCAG | |
| | ATCAGTGTGCAGCCTACATTCTCAGTGCAGCGGAATCTCCCTTTT | |
| | GAAAGAGCAACCGTTATGGCAGCATTCAGCGGGAACAATGAAGGA | |
| | CGGACATCCGACATGCGAACAGAAGTTATAAGAATGATGGAAAGT | |
| | GCAAAGCCAGAAGATTTGTCCTTCCAGGGGGGGGAGTCTTCGAGC | |
| | TCTCGGACGAAAAGGCAACGAACCCGATCGTGCCTTCCTTTGACA | |
| | TGAGTAATGAAGGGTCTTATTTCTTCGGAGACAATGCAGAGGAGT | |
| | ATGACAGTTGAGGAAAAATACCCTTGTTTCTACT | |
| HA P6 | AGCAAAAGCAGGGGAAAATAAAAGCAACAAAAATGAAGGCAATAC | 9 |
| | TAGTAGTTCTGCTATATACATTTGCAACCGCAAATGCAGACACAT | |
| | TATGTATAGGTTATCATGCGAACAATTCAACAGACACTGTAGACA | |
| | CAGTACTAGAAAAGAATGTAACAGTAACACACTCTGTTAACCTAT | |
| | TGGAGGATAAGCATAACGGTAAGCTATGCAAACTGAGAGGCGTAG | |
| | CACCATTGCATCTAGGTAAGTGTAATATAGCCGGATGGATTCTAG | |
| | GGAATCCCGAATGCGAATCACTATCAACCGCTAGCTCATGGTCAT | |
| | ACATAGTCGAAACACCATCAAGCGATAACGGTACATGTTATCCCG | |
| | GAGACTTTATCGATTACGAAGAGCTTAGAGAGCAATTGTCTAGCG | |
| | TAAGCTCATTCGAAAGATTCGAAATTTTTCCGAAAACTAGCTCAT | |
| | GGCCTAATCACGATAGTAATAAAGGCGTAACTGCCGCATGCCCAC | |
| | ACGCCGGAGCTAAATCATTCTATAAGAATCTGATTTGGTTAGTGA | |
| | AAAAAGGGAATTCATATCCGAAACTATCTAAATCATACATTAACG | |
| | ATAAGGGTAAGGAGGTACTAGTGTTGTGGGGGATACACCATCCAT | |
| | CAACTAGCGCCGATCAGCAATCATTGTATCAGAACGCAGACGCAT | |
| | ACGTATTCGTAGGGTCTAGTAGATACTCTAAAAAATTTAAACCCG | |
| | AAATCGCAATTAGACCGAAAGTGAGAGACCAAGAGGGTAGAATGA | |
| | ATTACTATTGGACACTAGTCGAACCAGGCGATAAGATTACATTCG | |
| | AAGCGACAGGGAATCTAGTCGTACCGAGATACGCATTCGCAATGG | |
| | AGAGAAACGCCGGATCCGGAATTATTATTAGCGATACACCCGTAC | |
| | ACGATTGCAATACTACATGTCAGACACCAAAAGGCGCAATTAATA | |
| | CTAGTCTGCCATTTCAGAATATACACCCAATTACAATCGGTAAGT | |
| | GTCCAAAATACGTTAAGTCAACTAAGTTGAGACTCGCAACAGGGT | |
| | TGAGAAATATACCGTCAATTCAATCTAGGGGGTTGTTCGGAGCAA | |
| | TCGCAGGGTTTATCGAAGGGGGGTGGACAGGTATGGTTGACGGAT | |
| | GGTACGGATACCATCATCAAAACGAACAGGGATCCGGATACGCAG | |
| | CCGATCTGAAAAGTACACAGAACGYTATAGACGAAATTACGAATA | |
| | AAGTGAATAGCGTAATCGAAAAAATGAATACGCAATTTACAGCCG | |
| | TAGGTAAGGAGTTTAATCATCTCGAAAAAAGGATTGAGAATCTGA | |
| | ATAAAAAAGTCGACGACGGATTCTTAGACATTTGGACTTATAACG | |
| | CCGAACTGTTAGTGTTACTCGAAAACGAAAGAACACTAGACTATC | |
| | ACGATTCAAACGTTAAGAATCTATACGAAAAAGTGAGATCGCAAT | |
| | TGAAAAATAACGCTAAAGAGATAGGGAATGGGTGTTTCGAATTCT | |
| | ATCATAAATGCGATAATACATGTATGGAATCCGTTAAAAACGGAA | |
| | CATACGATTACCCTAAGTATAGCGAAGAGGCTAAACTGAATAGGG | |
| | AAGAGATAGACGGAGTGAAACTCGAATCAACTAGGATITATCAGA | |
| | TACTCGCAATTTATAGTACGGTTGCCAGTTCATTGGTACTGGTAG | |
| | TCTCCCTGGGGGCAATCAGTTTCTGGATGTGCTCTAATGGGTCTC | |
| | TACAGTGTAGAATATGTATTTAACATTAGGATITCAGAAGCATGA | |
| | GAAAAACACCCTTGTTTCTACT | |
| | Y is C or T. | |
| HA P7 | AGCAAAAGCAGGGGAAAATAAAAGCAACAAAAATGAAGGCAATAC | 10 |
| | TAGTAGTTCTGCTATATACATTTGCAACCGCAAATGCAGACACAT | |
| | TATGTATAGGTTATCATGCGAACAATTCAACAGACACTGTAGACA | |
| | CAGTACTAGAAAAGAATGTAACAGTAACACACTCTGTTAACCTAT | |
| | TGGAGGATAAGCATAACGGTAAGCTATGCAAACTGAGAGGCGTAG | |
| | CACCATTGCATCTAGGTAAGTGTAATATAGCCGGATGGATTCTAG | |
| | GGAATCCCGAATGCGAATCACTATCAACCGCTAGCTCATGGTCAT | |
| | ACATAGTCGAAACACCATCAAGCGATAACGGTACATGTTATCCCG | |
| | GAGACTTTATCGATTACGAAGAGCTTAGAGAGCAATTGTCTAGCG | |
| | TAAGCTCATTCGAAAGATTCGAAATTTTTCCGAAAACTAGCTCAT | |
| | GGCCTAATCACGATAGTAATAAAGGCGTAACTGCCGCATGCCCAC | |
| | ACGCCGGAGCTAAATCATTCTATAAGAATCTGATTTGGTTAGTGA | |
| | AAAAAGGGAATTCATATCCGAAACTATCTAAATCATACATTAACG | |
| | ATAAGGGTAAGGAGGTACTAGTGTTGTGGGGGATACACCATCCAT | |
| | CAACTAGCGCCGATCAGCAATCATTGTATCAGAACGCAGACGCAT | |
| | ACGTATTCGTAGGGTCTAGTAGATACTCTAAAAAATTTAAACCCG | |
| | AAATCGCAATTAGACCGAAAGTGAGAGACCAAGAGGGTAGAATGA | |
| | ATTACTATTGGACACTAGTCGAACCAGGCGATAAGATTACATTCG | |
| | AAGCGACAGGGAATCTAGTCGTACCGAGATACGCATTCGCAATGG | |
| | AGAGAAACGCCGGATCCGGAATTATTATTAGCGATACACCCGTAC | |
| | ACGATTGCAATACTACATGTCAGACACCAAAAGGCGCAATTAATA | |
| | CTAGTCTGCCATTTCAGAATATACACCCAATTACAATCGGTAAGT | |
| | GTCCAAAATACGTTAAGTCAACTAAGTTGAGACTCGCAACAGGGT | |
| | TGAGAAATATACCGTCAATTCAATCTAGGGGGTTGTTCGGAGCAA | |
| | TCGCAGGGTTTATCGAAGGGGGGTGGACAGGTATGGTTGACGGAT | |
| | GGTACGGATACCATCATCAAAACGAACAGGGATCCGGATACGCAG | |
| | CCGAWCTGAAAAGTACACAGAACGYTATAGACGAAATTACGAATA | |
| | AAGTGAATAGCGTAATCGAAAAAATGAATACGCAATTTACAGCCG | |
| | TAGGTAAGGAGTTTAATCATCTCGAAAAAAGGATTGAGAATCTGA | |
| | ATAAAAAAGTCGACGACGGATTCTTAGACATTTGGACTTATAACG | |
| | CCGAACTGTTAGTGTTACTCGAAAACGAAAGAACACTAGACTATC | |
| | ACGATTCAAACGTTAAGAATCTATACGAAAAAGTGAGATCGCAAT | |
| | TGAAAAATAACGCTAAAGAGATAGGGAATGGGTGTTTCGAATTCT | |
| | ATCATAAATGCGATAATACATGTATGGAATCCGTTAAAAACGGAA | |
| | CATACGATTACCCTAAGTATAGCGAAGAGGCTAAACTGAATAGGG | |
| | AAGAGATAGACGGAGTGAAACTCGAATCAACTAGGATITATCAGA | |
| | TACTCGCAATTTATAGTACGGTTGCCAGTTCATTGGTACTGGTAG | |
| | TCTCCCTGGGGGCAATCAGTTTCTGGATGTGCTCTAATGGGTCTC | |
| | TACAGTGTAGAATATGTATTTAACATTAGGATTTCAGAAGCATGA | |
| | GAAAAACACCCTTGTTTCTACT | |
| | Y is C or T. W is T or A. | |
| A/California/ | agcaaaagcaggggaaaataaaagcaacaaaaatgaaggcaatac | 11 |
| 07/2009 | tagtagttctgctatatacatttgcaaccgcaaatgcagacacat | |
| M100/ | tatgtataggttatcatgcgaacaattcaacagacactgtagaca | |
| V6-HA- | cagtactagaaaagaatgtaacagtaacacactctgttaacctat | |
| Min | tggaggataagcataacagtaagctatgcaaactgagaggcgtag | |
| | caccattgcatctaggtaagtgtaatatagccggatggattctag | |
| | ggaatcccgaatgcgaatcactatcaaccgctagctcatggtcat | |
| | acatagtcgaaacaccatcaagcgataacggtacatgttatcccg | |
| | gagactttatcgattacgaagagcttagagagcaattgtctagcg | |
| | taagctcattcgaaagattcgaaatttttccgaaaactagctcat | |
| | ggcctaatcacgatagtagtaaaggcgtaactgccgcatgcccac | |
| | acgccggagctaaatcattctataagaatctgatttggttagtga | |
| | aaaaagagaattcatatccgaaactatctaaatcatacattaacg | |
| | ataagggtaaggaggtactagtgttgtgggggatacaccatccat | |
| | caactagcgccgatcagcaatcattgtatcagaacgcagacgcat | |
| | acgtattcgtagggtctagtagatactctaaaaaatttaaacccg | |
| | aaatcgcaattagaccgaaagtgagaggccaagagggtagaatga | |
| | attactattggacactagtcgaaccaggcgataagattacattcg | |
| | aagcgacagggaatctagtcgtaccgagatacgcattcgcaatgg | |
| | agagaaacgccggatccggaattattattagcgatacacccgtac | |
| | acgattgcaatactacgtgtcagacaccaaaaggcgcaattaata | |
| | ctagtctgccatttcagaatatacacccaattacaatcggtaagt | |
| | gtccaaaatacgttaagtcaactaagttgagactcgcaacagggt | |
| | tgagaaatacaccgtcaattcaatctagggggttgttcggagcaa | |
| | tcgcagggtttatcgaaggggggtggacaggtatggttgacggat | |
| | ggtacggataccatcatcaaaacgaacagggatccggatacgcag | |
| | ccgaactgaaaagtacacagaacgctatagacgaaattacgaata | |
| | aagtgaatagcgtaatcgaaaaaatgaatacgcaatttacagccg | |
| | taggtaaggagtttaatcatctcgaaaaaaggattgagaatctga | |
| | ataaaaaagtcgacgacggattcttagacatttggacttataacg | |
| | ccgaaatgttagtgttactcgaaaacgaaagaacactagactatc | |
| | acgattcaaacgttaagaatctatacgaaaaagtgagatcgcaat | |
| | tgaaaaataacgctaaagagatagggaatgggtgtttcgaattct | |
| | atcataaatgcgataatacatgtatggaatccgttaaaaacggaa | |
| | catacgattaccctaagtatagcgaagaggctaaactgaataggg | |
| | aagagatagacggagtggaacttgaatcaactaggatttatcaga | |
| | tactcgcaatttatagtacggttgccagttcattggtactggtag | |
| | tctccctgggggcaatcagtttctggatgtgctctaatgggtctc | |
| | tacagtgtagaatatgtatttaacattaggatttcagaagcatga | |
| | gaaaaacacccttgtttctact | |
| A/California/ | agcaaaagcaggggtttaaaATGAATCCAAACCAAAAGATAATAA | 12 |
| 07/2009M100/ | CCATTGGTTCGGTCTGTATGACAATTGGAATGGCTaacttaatat | |
| V6-NA-Min | tacaaattggaaacataatctcaatatggattagccactcaatcc | |
| | aattggggaatcagaatcaaatcgaaacatgcaatcaatccgtaa | |
| | ttacatacgagaataatacttgggtgaatcagacatacgttaaca | |
| | tatcgaatactaatttcactgccggacaatccgtcgtgagtgtga | |
| | aactagccggtaatagtagtctatgtcccgttagcggatgggcta | |
| | tatactctaaagacaatagcgttagaatcggatctaaaggcgacg | |
| | tattcgttatacgcgaaccattcataagttgtagtccattagagt | |
| | gtaggactttttttctgacacagggcgcactattgaacgataagc | |
| | attctaacggtacaatcaaagataggtcaccatatagaacactaa | |
| | tgtcatgtccgataggcgaagtgcctagtccatacaatagtagat | |
| | tcgaatccgtcgcttggtccgctagcgcatgccatgacggtatta | |
| | attggttgacaatcgggattagcggacccgataacggcgcagtcg | |
| | ccgtacttaagtataacggtataattaccgatactattaagagtt | |
| | ggcgaaataatatattgcgaacacaggaatccgaatgcgcatgcg | |
| | ttaacggatcatgttttaccgttatgactgacggaccatctaacg | |
| | ggcaagcgtcatataagatttttagaatcgaaaaaggtaagatag | |
| | tgaaatccgtcgaaatgaacgctcctaattatcattacgaagagt | |
| | gctcatgttatcccgattctagcgaaattacatgcgtatgtagag | |
| | acaattggcacggatctaatagaccttgggtgtcattcaatcaga | |
| | atctagagtatcaaatcgggtatatatgctcagggatattcggag | |
| | acaatcctagacctaacgataagacagggtcatgcggaccagtga | |
| | gttctaacggcgctaacggcgttaaggggtttagtttcaaatacg | |
| | gtaacggcgtatggatagggagaactaagtcaatctctagtagaa | |
| | acggattcgaaatgatatgggaccctaacggatggaccggaaccg | |
| | ataataatttttcgattaaacaggatatcgtagggattaacgaat | |
| | ggtcagggtatagcggatcattcgtacagcatccagagttaaccg | |
| | gactcgattgcatacgaccatgtttttgggtcgaactgattaggg | |
| | ggagaccgaaagagaatactatatggactagcgggagcagcatat | |
| | ccttttgtggtgtaaacagtgacactgtgggttggtcttggccag | |
| | acggtgctgagttgccatttaccattgacaagtaatttgttcaaa | |
| | aaactccttgtttctact | |
| A/CA/07/ | agcaaaagcaggggaaaataaaagcaacaaaaATGAAGGCAATAC | 13 |
| 2009- | TAGTAGTTCTGCTATATACATTTGCAACCGCAAATGCAGACACAT | |
| HA- | TATGTATAGGTTATCATGCGAACAATTCAACAGACACTGTAGACA | |
| Min- | CAaTACTAGAAAAGAATGTAACAGTAACACACTCTGTTAACCTAT | |
| Vero17P | TGGAGGATAAGCATAACGGTAAGCTATGCAAACTGAGAGGCGTAG | |
| | CACCATTGCATCTAGGTAAGTGTAATATAGCCGGATGGATTCTAG | |
| | GGAATCCCGAATGCGAATCACTATCAACCGCTAGCTCATGGTCAT | |
| | ACATAGTCGAAACACCATCAAGCGATAACGGTACATGTTATCCCG | |
| | GAGACTTTATCGATTACGAAGAGCTTAGAGAGCAATTGTCTAGCG | |
| | TAAGCTCATTCGAAAGATTCGAAATTTTTCCGAAAACTAGCTCAT | |
| | GGCCTgATCACGATAGTAATAAAGGCGTAACTGCCGCATGCCCAC | |
| | ACGCCGGAGCTAAATCATTCTATgAGAATCTGgTTTGGTTAGTGA | |
| | AAAAAGGGAATTCATATCCGAAACTATCTAAATCATACATTAACG | |
| | ATAAGGGTAAGGAGGTACTAGTGTTGTGGGGGATACACCATCCAT | |
| | CAACTAGCGCCGATCAGCAATCATTGTATCAGAACGCAGACGCAT | |
| | ACGTATTCGTAGGGTCTAGTAGATACTCTAAAAAATTTAAACCCG | |
| | AAATCGCAATTAGACCGAAAGTGAGAGgCCAAGAGGGTAGAATGA | |
| | ATTACTATTGGACACTAGTCGAACCAGGCGATAAGATTACATTCG | |
| | AAGCGACAGGGAATCTAGTCGTACCGAGATACGCATTCGCAATGG | |
| | AGAGAAACGCCGGATCCGGAATTATTATTAGCGATACACCCGTAC | |
| | ACGATTGCAATACTACATGTCAGACACCAgAAGGCGCAATTAATA | |
| | CTAGTCTGCCATTTCAGAATATACACCCAATTACAATCGGTAAGT | |
| | GTCCAAAATACGTTAAGTCAACTAAGTTGAGACTCGCAACAGGGT | |
| | TGAGAAATATACCGTCAATTCAATCTAGGGGGTTGTTCGGAGCAA | |
| | TCGCAGGGTTTATCGAAGGGGGGTGGACAGGTATGGTTGACGGAT | |
| | GGTACGGATACCATCATCAAAACGAACAtGGATCCGGATACGCAG | |
| | CCGAaCTGAAAAGTACACAGAACGCTATAGACGAAATTACGAATA | |
| | AAGTGAATAGCGTAATCGAAAAAATGAATACGCAATTTACAGCCG | |
| | TAGGTAAGGAGTTTAATCATCTCGAAAAAAGGATTGAGAATCTGA | |
| | ATAAAAAAGTCGACGACGGATTCTTAGACATTTGGACTTATAACG | |
| | CCGAACTGTTAGTGTTACTCGAAAACGAAAGAACACTAGACTATC | |
| | ACGATTCAAACGTTAAGAATCTATACGAAAAAGTGAGATCGCAAT | |
| | TGAAAAATAACGCTAAAGAGATAGGGAATGGGTGTTTCGAATTCT | |
| | ATCATAAATGCGATAATACATGTATGGAATCCGTTAAAAACGGAA | |
| | CATACGATTACCCTAAGTATAGCGAAGAGGCTAAACTGAATAGaG | |
| | AAGAGATAGACGGAGTGgAACTCGAgTCAACTAGGATTTATCAGA | |
| | TACTCGCAATTTATAGTACGGTTGCCAGTTCATTGGTACTGGTAG | |
| | TCTCCCTGGGGGCAATCAGTTTCTGGATGTGCTCTAATGGGTCTC | |
| | TACAGTGTAGAATATGTATTTAAcattaggatttcagaagcatga | |
| | gaaaaacacccttgtttctact | |
| A/CA/07/ | agcaaaagcaggggtttaaaATGAATCCAAACCAAAAGATAATAA | 14 |
| 2009- | CCATTGGTTCGGTCTGTgTGACAATTGGAATGGCTAACTTAATAT | |
| NA- | TACAAATTGGAAACATAATCTCAATATGGATTAGCCACTCAATCC | |
| Min- | AATTGGGGAATCAGAATCAAATCGAAACATGCAATCAATCCGTAA | |
| Vero17P | TTACATACGAGAATAATACTTGGGTGAATCAGACATACGTTAACA | |
| | TATCGAATACTAATTTCaCTGCCGGACcATCCGTCGTGAGTGTGA | |
| | AACTAGCCGGTAATAGTAGTCTATGTCCCGTTAGCGGATGGGCTA | |
| | TATACTCTAAAGACAATAGCGTTAGAATCGGATCTAAAGGCGACG | |
| | TATTCGTTATACGCGAACCATTCATAAGTIGTAGTCCATTAGAGT | |
| | GTAGGACTTTTTTTCTGACACAGGGCGaACTATTGAACGATAAG | |
| | CATTCTAACGGTACAATCAAAGATAGGTCACCATATAGAACACTA | |
| | ATGTCATGTCCGATAGGCGAAGTGCCTAGTCCATACAATAGTAaA | |
| | TTCGAATCCGTCGCTTGGTCCGCTAGCGCATGCCATGACGGTATT | |
| | AATTGGTTGACAATCGGGATTAGCGGACCCGATAACGGCGCAGTC | |
| | GCCGTACTTAAGTATAACGGTATAATTACCGATACTATTAAGAGT | |
| | TGGCGAAATAATATATTGCGAACACAGGAATCCGAATGCGCATGC | |
| | GTTAACGGATCATGTTTTACCGTTATGACTGACGGACCATCTAAC | |
| | GGGCAAGCGTCATATAAGATTTTTAGAATCGAAAAAGGTAAGATA | |
| | GTGAAATCCGTCGAAATGAACGCTCCTAATTATCATTACGAAGAG | |
| | TGCTCATGTTATCCCGATTCTAGCGAAATTACATGCGTATGTAGA | |
| | GACAATTGGCACGGATCTAATAGACCTTGGGTGTCATTCAATCAG | |
| | AATCTAGAGTATCAAATCGGGTATATATGCTCAGGGATATTCGGA | |
| | GACAATCCTAGACCTAACGATAAGACAGGGTCATGCGGACCAGTG | |
| | AGTTCTAACGGCGCTAACGGCGTTAAGGGGTTTAGTTTCAAATAC | |
| | GGTAACGGCGTATGGATAGGGAGAACTAAGTCAATCTCTAGTAGA | |
| | AACGGATTCGAAATGATATGGGACCCTAACGGATGGACCGGAACC | |
| | GATAATAATTTTTCGATTAAACAGGATATCGTAGGGATTAACGAA | |
| | TGGTCAGGGTATAGCGGATCATTCGTACAGCATCCAGAGTTAACC | |
| | GGACTCGATTGCATACGACCATGTTTTTGGGTCGAACTGATTAGG | |
| | GGGAGACCGAAAGAGAATACTATATGGACTAGCGGGAGCAGCATA | |
| | TCCTTTTGTGGTGTAAACAGTGACACTGTGGGTTGGTCTTGGCCA | |
| | GACGGTGCTGAGTTGCCATTTACCATTGACAAGTAAtttgttcaa | |
| | aaaactccttgtttctact | |

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Sialyltransferase Expression in Breast Cancer

### TCGA Analysis

To determine expression of key sialyltransferase genes in human breast cancers as a surrogate for sialic acid exposure on the cell surface, the invasive breast cancer data set was accessed via xenabrowser.net (University of Santa Cruz) on May 14, 2021.

Data included gene expression data from TCGA for cancer tissue and from TCGA's Genotype-Tissue Expression project (GTEx) for normal tissue controls (total n=1278). Normalized gene expression levels of ST3 Beta-Galactoside Alpha-2,3-Sialyltransferase 1 (ST3GAL1) and ST6 Beta-Galactoside Alpha-2,6-Sialyltransferase 1 (ST6GAL1), enzymes needed in the final steps of α2,3 and α2,6 sialic acids linkage, respectively, as well as ERBB2, the gene encoding Her2 in normal tissues (n=179), primary tumors (n=1092), and breast cancer metastases (n=7) were exported. Expression levels for each gene and cohort were plotted as violin plots and statistical significance was determined using Kruskal-Wallis test with Dunn's multiple comparison test.

To determine differences in sialyltransferase expression by Her-2 status, tumor and metastasis samples were combined into one cohort and then divided into Her2^{high} and Her2^{low} groups based on the highest expression value in the normal breast tissue cohort. Expression levels for both genes in each group were plotted as violin plots and statistical significance compared to normal tissues was determined using Kruskal-Wallis test with Dunn's multiple comparison test.

### Results

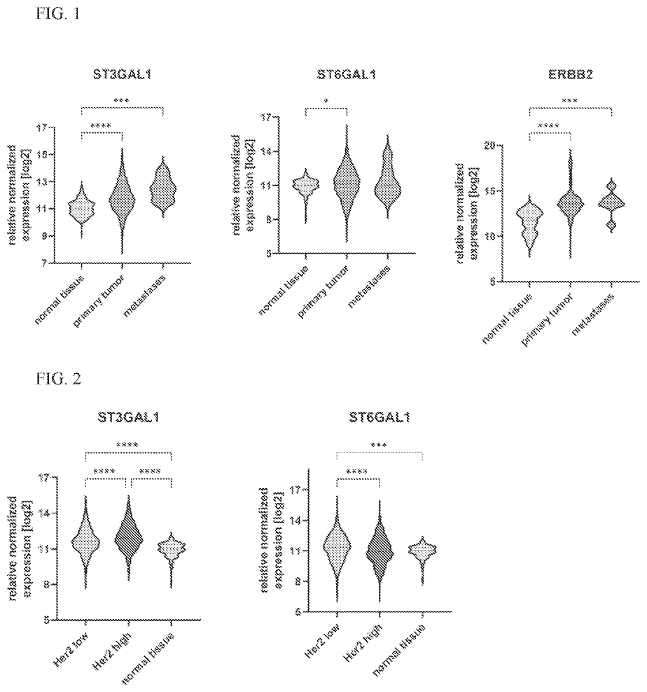

To establish sialic acid expression in human invasive breast cancer, gene expression of ST3Gal1 and ST6Gal1 in breast tumor and normal breast tissues was compared. The enzymes encoded by these two gene catalyze the terminal α2,3 and α2,6 sialic acid linkage in glycosylation patterns of surface glycoproteins. ST3Gal1 gene expression was significantly upregulated in both primary breast cancer and metastases as compared to normal breast tissues (FIG. 1). In primary tumors, also ST6Gal1 was moderately upregulated (p=0.014).

To determine whether sialyltransferase expression differed based on Her2 expression, tumor samples were divided into Her2h^{igh} and Her2^{low} expressors based on the maximum ERBB2 gene expression value in normal control tissue (max=13.66 log 2(norm_count+1), FIG. 2). Based on this cut-off, 489 tissues fell into the Her2^{low} category, 609 tumor tissues were categorized as Her2^{low}.

ST3Gal1 expression was significantly upregulated in both Her2^{high} and Her2^{low} tumors as compared to normal tissue with further enrichment in Her2^{high} tumors. In contrast, ST6Gal1 expression was higher in Her2^{low} tumors, while Her2^{low} tumors did not demonstrate significant enrichment of expression as compared to normal tissues. Of note, even in this subset, 20.65% (101/489) of tumors had a higher expression level of ST6Gal1 than observed across normal breast tissues.

### Summary and Conclusions

In this experiment, sialyltransferase expression as a surrogate for α2,3-linked and α2,6-linked sialic acids, essential for influenza virus entry into host cells, was assessed in a cohort of normal and cancerous breast tissues. Gene expression of both enzymes was significantly increased across the tumor cohort as compared to normal tissues. ST3Gal1 expression was elevated in both Her2^{high} and Her2^{low} tumors, while ST6Gal1 was moderately, but significantly elevated in Her2^{low} tumors.

Increased presence of α2,3-linked and α2,6-linked sialic acids as well as increased expression of the key sialyltransferases ST3Gal1 and ST6Gal1 have been previously observed in independent cohorts of breast cancer tissues in comparison to normal mammary tissue. The analyses in this study further corroborated the increased expression of both enzymes in breast cancer tissues.

Acknowledging that additional enzymes within the glycosylation pathway contribute to surface expose of α2,3-linked and α2,6-linked sialic acids and multiple cell-intrinsic factors impact productive replication of influenza virus in host cells, these data support the potential preferential infection of human breast cancer cells over healthy breast cancer tissue. In addition, this analysis may suggest no restriction of Her2^{high} or Her2^{low} breast cancer is required.

### Example 2

### Sialyltransferase Expression Across Tumor Types

### TCGA Analysis

To determine expression of sialyltransferase genes in different human cancers as a surrogate for sialic acid exposure on the cell surface, TCGA data was accessed and visualized using the Gene Expression Profiling Interactive Analysis (GEPIA) application (gepia.cancer-pku.cn/index.html; Tang et al., Nucleic Acid Res 2017, 10:1093).

The data set included 31 different tumor types and corresponding non-malignant tissues from both the original TCGA data set as well as the Genotype-Tissue Expression (GTEx) data set (Table 1). All TCGA-tracked tumor types were included in the analysis except mesothelioma and uveal melanoma, for which no normal control tissue data was available, therefore not allowing for an assessment whether sialyltransferases are differentially expressed in these tumors.

Gene expression plots for each of the two ST6 Beta-Galactoside Alpha-2,6-Sialyltransferases (ST6Gal1-2) and the 6 ST3 Beta-Galactoside Alpha-2,3-Sialyltransferases (ST3Gal1-6), that include individual data points and medians were downloaded directly using the “Expression DIY” tool. Expression levels expressed as transcripts per million (TPM) are shown in a log₂(TPM+1) scale. The tool was also used to calculate statistics: ANOVA was used to compare tumor and paired normal samples, a q value of 0.01 was selected as significance cutoff. In addition, a median fold change of 2 ([log₂FC=1] was chosen to highlight meaningful differences in tumors as compared to the respective control tissues. Using these parameters, the toll automatically highlights tumor types in which expression of the respective gene is significantly overexpressed by at least 2-fold in red, and those with significant repression by the same factor in green.

TABLE 1
Tumor types included in the analysis and sample sizes

| TCGA | | Tumor | Normal |
|---|---|---|---|
| abbreviation | Tumor type | tissues | tissues |
| ACC | Adrenocortical carcinoma | 77 | 128 |
| BLCA | Bladder Urothelial Carcinoma | 404 | 28 |
| BRCA | Breast invasive carcinoma | 1085 | 291 |
| CESC | Cervical squamous cell carcinoma | 306 | 13 |
| | and endocervical adenocarcinoma | | |
| CHOL | Cholangio carcinoma | 36 | 9 |
| COAD | Colon adenocarcinoma | 275 | 349 |
| DLBC | Lymphoid Neoplasm Diffuse Large | 47 | 337 |
| | B-cell Lymphoma | | |
| ESCA | Esophageal carcinoma | 182 | 286 |
| GBM | Glioblastoma multiforme | 163 | 207 |
| HNSC | Head and Neck squamous cell | 519 | 44 |
| | carcinoma | | |
| KICH | Kidney Chromophobe | 66 | 53 |
| KIRC | Kidney renal clear cell carcinoma | 523 | 100 |
| KIRP | Kidney renal papillary cell | 286 | 60 |
| | carcinoma | | |
| LAML | Acute Myeloid Leukemia | 173 | 70 |
| LGG | Brain Lower Grade Glioma | 518 | 207 |
| LIHC | Liver hepatocellular carcinoma | 369 | 160 |
| LUAD | Lung adenocarcinoma | 483 | 347 |
| LUSC | Lung squamous cell carcinoma | 486 | 338 |
| OV | Ovarian serous cystadenocarcinoma | 426 | 88 |
| PAAD | Pancreatic adenocarcinoma | 179 | 171 |
| PCPG | Pheochromocytoma and Paraganglioma | 182 | 3 |
| PRAD | Prostate adenocarcinoma | 492 | 152 |
| READ | Rectum adenocarcinoma | 92 | 318 |
| SARC | Sarcoma | 262 | 2 |
| SKCM | Skin Cutaneous Melanoma | 461 | 558 |
| STAD | Stomach adenocarcinoma | 408 | 211 |
| TGCT | Testicular Germ Cell Tumors | 137 | 165 |
| THCA | Thyroid carcinoma | 512 | 337 |
| THYM | Thymoma | 118 | 339 |
| UCEC | Uterine Corpus Endometrial Carcinoma | 174 | 91 |
| UCS | Uterine Carcinosarcoma | 57 | 78 |

To qualitatively rank order tumor types by expression of all 8 analyzed sialyltransferases, scores were assigned to fold change of expression in tumor as compared to their paired control tissues (Table 2). Scores were added up if statistically significant; no weighting was applied to specific sialyltransferase. The resulting total scores support rank ordering of tumor types which may be suitable for CodaLytic treatment, based on the assumption that sialyltransferase expression correlates with α2,3 and α2,6 sialic acid surface exposure.

TABLE 2
Scoring system based on fold change gene expression in
tumors as compared to paired normal control tissues.

| Score | Definition |
|---|---|
| +++ | >10-fold overexpression in tumor |
| ++ | 5-10-fold overexpression in tumor |
| + | 2-5-fold overexpression in tumor or expression in tumor |
| | without any expression in paired normal control tissue |
| 0 | expression in tumor between half and 2-fold of paired |
| | normal control tissue |
| − | 2-5-fold repression in tumor |
| −− | 5-10-fold repression in tumor |
| −−− | >10-fold repression in tumor |

### Results

Human influenza viruses, like the H1N1 influenza A virus CodaLytic is derived from, preferentially use α2,6-linked sialic acids for attachment and entry. Sialyltransferase ST6Gal1 and 2 create this specific terminal linkage.

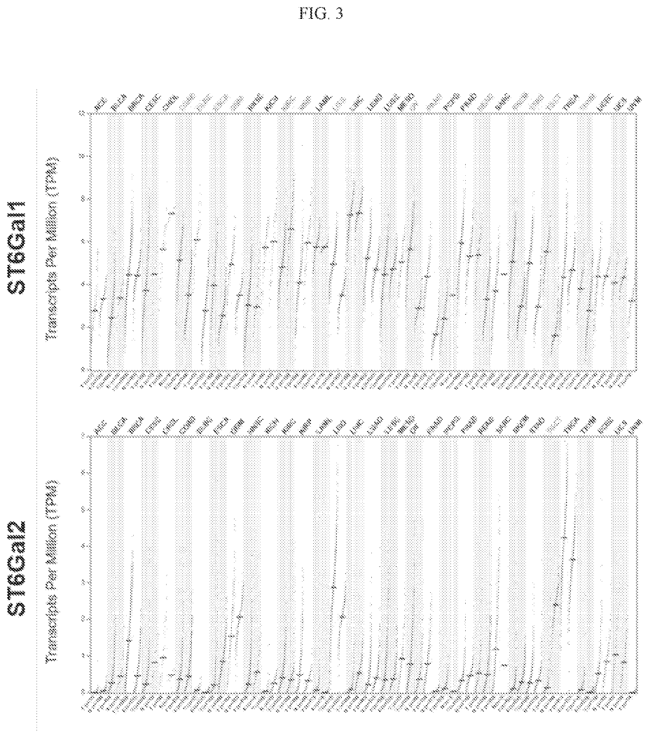

ST6Gal1 was expressed at higher levels across tumor and normal tissues than ST6Gal2 (FIG. 3, Table 3), in alignment with ST6Gal1 being the primary enzyme to catalyze this linkage. The enzyme was most dramatically overexpressed in testicular germ cell tumors (TGCT, 22.2-fold), diffuse large B cell lymphoma (DLBC, 11.6-fold), pancreatic adenocarcinoma (PAAD, 9.1-fold) and ovarian serous cystadenocarcinoma (OV, 7.7-fold) as compared to the respective matched control tissues. In addition, ST6Gal1 was significantly overexpressed in skin cutaneous melanoma (SKCM), various tumors of the gastrointestinal tract (stomach (STAD), rectal (READ), colon (COAD), and esophageal (ESCA) carcinomas), both lower grade glioma (LGG) and glioblastoma (GBM) as well as thymoma (THYM). In contrast, ST6Gal1 was significantly downregulated in clear cell (KIRC, 0.29-fold) and papillary cell (KIRP, 0.26-fold) kidney cancer. Of note, ST6Gal1 expression was highest overall in hepatocellular carcinoma (LIHC), however expression in normal liver hepatocytes was equally high.

ST6Gal2 was detected at much lower transcript number across histologies with strongest expression in both malignant (THCA) and normal thyroid tissue, malignant (LGG, GBM) and normal brain tissues, normal testicular tissue, and invasive breast cancer tissue (BRCA). Statistical analysis revealed no significant overexpression in any tumor type, despite a 36-fold median overexpression in pancreatic adenocarcinoma (PAAD) and 4.5-fold overexpression in invasive breast cancer (BRCA). ST6Gal2 expression was significantly downregulated in testicular germ cell tumors (TGCT, 0.02-fold).

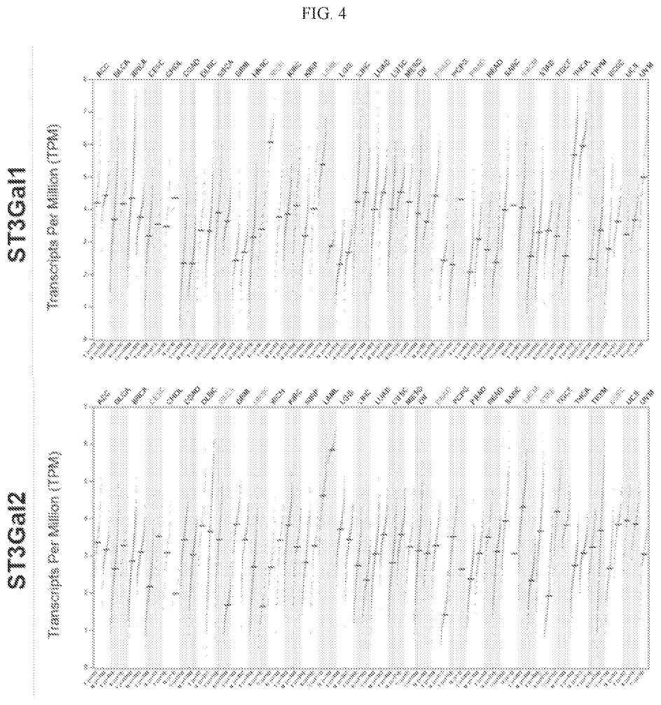

In addition to α2,6-linked sialic acids, influenza viruses can also use terminal α2,3-linked sialic acids for attachment and entry. Therefore, differential gene expression of all six ST3Gal's was analyzed (FIGS. 4-6, Table 3). ST3Gal1 was significantly overexpressed in chromophobe kidney cancer (KICH, 5.4-fold), in which median expression was the highest of all tumor types in the dataset (FIG. 4, top panel). In addition, ST3Gal1 was significantly overexpressed in acute myeloid leukemia (LAML, 6.4-fold), pancreatic adenocarcinoma (PAAD, 4.6-fold) and melanoma (SKCM, 3.2-fold).

Overall, ST3Gal2 expression was lower than for ST3Gal1 and fairly uniform, with notable exceptions being bone marrow and acute myeloid leukemia cells with highest median expression overall and normal pancreas with particularly low expression (FIG. 2, lower panel). When compared to paired normal control tissues, ST3Gal2 was moderately, but significantly upregulated in pancreatic cancer (PAAD, 5.2-fold), melanoma (SKCM, 4.7-fold), esophageal carcinoma (ESCA, 4.4-fold), gastric cancer (STAD), 4.2-fold), and squamous cell carcinoma of the head and neck (HNSC, 2.6-fold).

ST3Gal3 was primarily not differentially expressed or expressed at lower levels in tumors as compared to their respective normal control tissues (FIG. 3, top panel). Among the seven tumor types with this repression were gynecological cancers, i.e. endometrial cancer (UCEC, 0.26-fold), cervical cancers (CESC, 0.28-fold) and ovarian serous cystadenocarcinoma (OV, 0.36-fold), and lower gastrointestinal tract tumors, i.e. colon (COAD, 0.19-fold) and rectal (READ, 0.16-fold) adenocarcinoma. the only overexpression pattern was observed in diffuse large B cell lymphoma when compared to normal blood (DLBC, 3.1-fold).

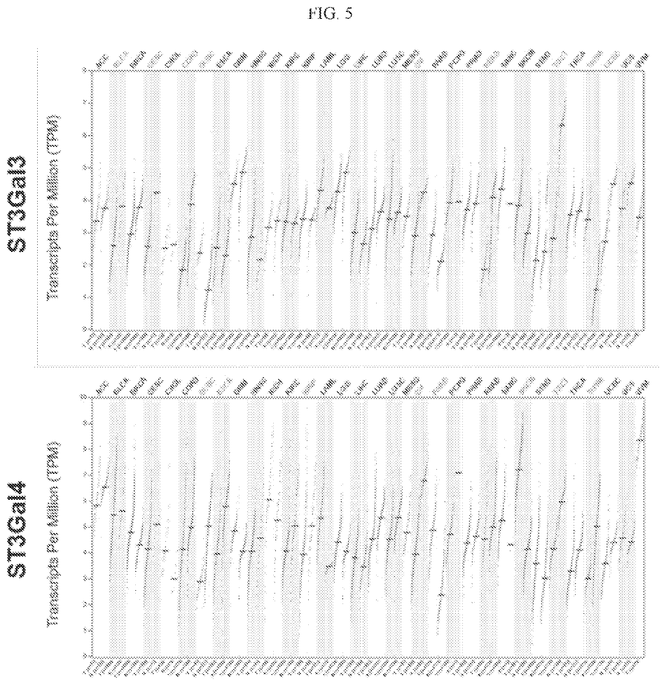

ST3Gal4 was significantly overexpressed in two tumor types (FIG. 5, lower panel). In pancreatic adenocarcinoma (PAAD, 6.7-fold) this fold change was primarily driven by the lowest overall expression levels in normal pancreas; in melanoma (SKCM, 8.8-fold) this was driven by strong expression in the malignant tissue. Of note, the highest median expression in any tumor type was observed in uveal melanoma; no control tissue samples are available for fold change calculations for this tumor type.

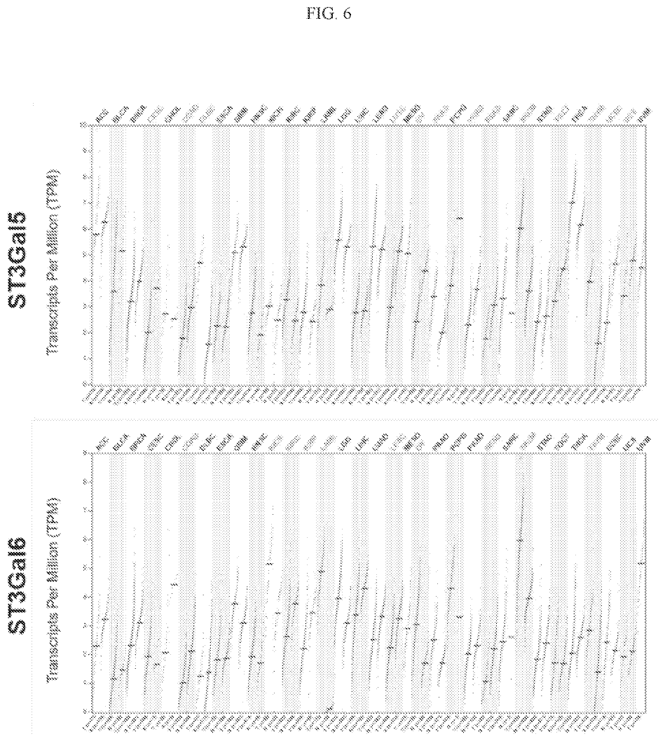

ST3Gal5 expression was highly variable across tissues (FIG. 6, top panel). This gene was significantly overexpression in comparison to control tissues in 4 different tumor types, including diffuse large B cell lymphoma (DLBC, 12.9-fold) and the solid tumor types thymoma (THYM, 7.4-fold), melanoma (SKCM, 5.8-fold) and pancreatic adenocarcinoma (PAAD, 3.2-fold). Of note, in thymoma and melanoma, median absolute expression levels were one of the highest across all tissues included in the data set.

ST3Gal6 was significantly overexpressed in melanoma of the skin (SKCM, 4.3-fold) and had high median expression levels in uveal melanoma (FIG. 6, lower panel). Other tumor types with relative overexpression as compared to control tissues include acute myeloid leukemia (LAML, 358-fold), thymoma (THYM, 3.8-fold), serous ovarian cancer (OV, 3.2-fold) and chromophobe kidney cancer (KICH, 3.5-fold). Interestingly, ST6Gal6 was expressed to significantly lower levels in the two other renal cancer subtypes included in the data set as compared to normal kidney (KIRC, 0.41-fold and KIRP, 0.37-fold).

To look at sialylation pattern more holistically, scores were assigned for each sialyltransferase and tumor type based on expression fold change over their paired control tissues and these scores were added up (Table 3). Based on this data aggregation, pancreatic adenocarcinomoa (PAAD) and melanoma (SKCM) showed the strongest overall sialyltransferase overexpression with a score of +8. Importantly, in both of these tumor types, ST6Gal1 overexpression contributed to this score, which represents the key enzyme catalyzing the α2,6 sialic acids linkage preferred by human influenza viruses. Additional tumor types that can be grouped into the top tier of relative tumor overexpression of sialyltransferase included the hematological malignancies diffuse large B cell lymphoma (DLBC, total score+6) and acute myeloid leukemia (LAML, +5) as well as the thymoma (THYM, +5), a rare tumor type originating within epithelial cells of the thymus, a lymphoid organ responsible for the development and maturation of cell-mediated immunologic functions.

TABLE 3
Differential expression of sialyltransferases in tumors as compared to their paired normal tissues (FC, fold change) and expression
scoring as described in Table 2. Bold font indicates statistical significance. n/c, cannot be calculated (divisor is 0).

| TCGA | ST6GAL1 | | ST6GAL2 | | ST3GAL1 | | ST3GAL2 | | ST3GAL3 | | ST3GAL4 | | ST3GAL5 | | ST3GAL6 | | Total |
|---|---|---|---|---|---|---|---|---|---|---|---|---|---|---|---|---|---|
| Abbreviation | FC | Score | FC | Score | FC | Score | FC | Score | FC | Score | FC | Score | FC | Score | FC | Score | score |
| ACC | 0.650 | 0 | 0.000 | −−− | 0.854 | 0 | 1.160 | 0 | 0.735 | 0 | 0.605 | 0 | 0.718 | 0 | 0.467 | − | 0 |
| BLCA | 0.470 | − | 0.571 | 0 | 0.697 | 0 | 0.609 | 0 | 0.386 | − | 0.892 | 0 | 0.320 | − | 0.693 | 0 | −1 |
| BRCA | 1.024 | 0 | 4.486 | + | 1.548 | 0 | 0.832 | 0 | 0.531 | 0 | 1.419 | 0 | 0.559 | 0 | 0.524 | 0 | 0 |
| CESC | 0.571 | 0 | 0.221 | − | 0.760 | 0 | 0.336 | − | 0.279 | − | 0.507 | 0 | 0.248 | − | 1.306 | 0 | −3 |
| CHOL | 0.310 | − | 2.410 | + | 0.524 | 0 | 2.524 | + | 0.913 | 0 | 2.283 | + | 1.178 | 0 | 0.154 | −− | 0 |
| COAD | 3.318 | + | 0.800 | 0 | 1.015 | 0 | 1.365 | 0 | 0.191 | −− | 0.546 | 0 | 0.359 | − | 0.309 | − | −3 |
| DLBC | 11.566 | +++ | n/c | + | 1.014 | 0 | 1.121 | 0 | 3.112 | + | 0.202 | − | 12.902 | +++ | 0.850 | 0 | +6 |
| ESCA | 2.998 | + | 0.190 | −− | 1.228 | 0 | 4.425 | + | 1.229 | 0 | 0.268 | − | 1.046 | 0 | 0.962 | 0 | +1 |
| GBM | 2.910 | + | 0.601 | 0 | 0.817 | 0 | 1.367 | 0 | 0.776 | 0 | 1.785 | 0 | 0.850 | 0 | 1.672 | 0 | +1 |
| HNSC | 1.067 | 0 | 0.362 | − | 0.840 | 0 | 2.587 | + | 1.802 | 0 | 0.682 | 0 | 2.075 | + | 1.233 | 0 | +1 |
| KICH | 0.825 | 0 | 0.105 | −− | 5.239 | ++ | 0.565 | 0 | 0.854 | 0 | 1.755 | 0 | 1.546 | 0 | 3.486 | + | +3 |
| KIRC | 0.286 | − | 1.185 | 0 | 0.828 | 0 | 1.556 | 0 | 1.039 | 0 | 0.491 | − | 1.936 | 0 | 0.413 | − | −2 |
| KIRP | 0.262 | − | 1.560 | 0 | 0.533 | 0 | 0.705 | 0 | 1.023 | 0 | 0.451 | − | 1.330 | 0 | 0.366 | − | −3 |
| LAML | 0.995 | 0 | n/c | + | 6.425 | ++ | 0.412 | − | 1.495 | 0 | 3.924 | + | 2.064 | + | 357.750 | +++ | +5 |
| LGG | 2.925 | + | 1.991 | 0 | 0.737 | 0 | 1.235 | 0 | 0.649 | 0 | 1.304 | 0 | 1.196 | 0 | 1.919 | 0 | +1 |
| LIHC | 0.948 | 0 | 0.133 | −− | 0.815 | 0 | 1.383 | 0 | 1.334 | 0 | 1.311 | 0 | 0.942 | 0 | 0.503 | 0 | 0 |
| LUAD | 1.458 | 0 | 0.516 | 0 | 0.694 | 0 | 0.667 | 0 | 0.663 | 0 | 0.556 | 0 | 1.087 | 0 | 0.524 | 0 | 0 |
| LUSC | 0.833 | 0 | 0.931 | 0 | 0.686 | 0 | 0.555 | 0 | 0.851 | 0 | 0.547 | 0 | 0.201 | − | 0.439 | − | −2 |
| OV | 7.687 | ++ | 2.500 | + | 1.213 | 0 | 1.064 | 0 | 0.359 | − | 0.131 | −− | 0.220 | − | 3.222 | + | −1 |
| PAAD | 9.106 | ++ | 36.000 | +++ | 4.623 | + | 5.235 | ++ | 1.985 | 0 | 6.709 | ++ | 3.188 | + | 2.071 | + | +8 |
| PCPG | 0.416 | − | 3.500 | + | 0.209 | − | 1.998 | 0 | 0.970 | 0 | 0.187 | −− | 0.157 | −− | 2.114 | + | 0 |
| PRAD | 1.533 | 0 | 0.658 | 0 | 0.426 | − | 0.574 | 0 | 0.873 | 0 | 0.828 | 0 | 0.337 | − | 0.773 | 0 | −2 |
| READ | 4.572 | + | 1.125 | 0 | 1.386 | 0 | 1.349 | 0 | 0.164 | −− | 0.712 | 0 | 0.323 | − | 0.298 | − | −3 |
| SARC | 0.364 | − | 1.881 | 0 | 0.738 | 0 | 1.943 | 0 | 1.217 | 0 | 1.911 | 0 | 1.506 | 0 | 0.525 | 0 | 0 |
| SKCM | 4.735 | + | 0.333 | − | 3.176 | + | 4.674 | + | 1.934 | 0 | 8.826 | ++ | 5.750 | ++ | 4.255 | + | +8 |
| STAD | 4.572 | + | 0.800 | 0 | 0.963 | 0 | 4.161 | + | 0.789 | 0 | 1.548 | 0 | 0.836 | 0 | 0.606 | 0 | +2 |
| TGCT | 22.245 | +++ | 0.021 | −−− | 1.629 | 0 | 1.311 | 0 | 0.077 | −−− | 0.274 | − | 0.396 | − | 1.027 | 0 | −5 |
| THCA | 0.782 | 0 | 1.557 | 0 | 0.829 | 0 | 0.770 | 0 | 0.915 | 0 | 0.541 | 0 | 1.815 | 0 | 0.619 | 0 | 0 |
| THYM | 2.221 | + | n/c | + | 0.497 | − | 0.715 | 0 | 7.037 | ++ | 0.225 | − | 7.384 | ++ | 3.785 | + | +5 |
| UCEC | 0.991 | 0 | 0.557 | 0 | 0.520 | 0 | 0.400 | − | 0.258 | − | 0.547 | 0 | 0.175 | −− | 1.285 | 0 | −4 |
| UCS | 0.835 | 0 | 1.333 | 0 | 0.713 | 0 | 1.074 | 0 | 0.564 | 0 | 1.127 | 0 | 0.366 | − | 0.846 | 0 | −1 |

The second tier of tumor types with moderate aggregated sialyltransferase overexpression included chromophobe kidney cancer (KICH, +3), gastric cancer (STAD, +2), esophageal cancer (ESCA, +1), squamous cell carcinoma of the head and neck (HNSC, +1) and both glioblastoma multiforme (GBM, +1) and lower grade glioma (LGG, +1). With the exception of chromophobe kidney cancer, all of these tumor types overexpressed ST6Gal1 in comparison to their paired control tissues.

All other tumor types had total scores of 0 or lower, indicating no consistent overexpression pattern of sialyltransferases or relative decreases in expression for specific enzymes. Notably, this included ovarian serous cystadenocarcinoma (OV), testicular cancer (TGCT), and colorectal adenocarcinomas (COAD and READ), in which ST6Gal1 was significantly overexpressed relative to the respective normal control tissues.

### Summary and Conclusions

In this experiment, sialyltransferase expression as a surrogate for α2,3-linked and α2,6-linked sialic acids, essential for influenza virus entry into host cells, was assessed in a publicly available data set that includes tumor and paired normal tissues across a range of histologies. Different sialyltransferases show different patterns of overexpression and repression across tumor types. These enzymes were upregulated most consistently in pancreatic adenocarcinoma and melanoma as well as diffuse large B cell lymphoma, acute myeloid leukemia and thymoma, when compared to their paired control tissues (Table 4). Importantly, relative overexpression of ST6Gal1, the primary enzyme catalyzing the α2,6 sialic acid linkage human influenza viruses prefer for attachment and entry, generally contributed to the high overall expression scores in all of these tumors except in acute myeloid leukemia.

TABLE 4
Top tier indications with highest differential expression across eight sialyltransferases in tumors as compared to their paired
normal tissues (FC, fold change). Bold font indicates statistical significance. n/c, cannot be calculated (divisor is 0).

| Tumor types with | Enzymes catalyzing | | | | | | | | | | | | | | | | |
|---|---|---|---|---|---|---|---|---|---|---|---|---|---|---|---|---|---|
| high potential for | α2,6 sialic acid linkage | | | | Enzymes catalyzing α2,3 sialic acid linkage | | | | | | | | | | | | |
| local infectivity | ST6GAL1 | | ST6GAL2 | | ST3GAL1 | | ST3GAL2 | | ST3GAL3 | | ST3GAL4 | | ST3GAL5 | | ST3GAL6 | | Total |
| with CodaLytic | FC | Score | FC | Score | FC | Score | FC | Score | FC | Score | FC | Score | FC | Score | FC | Score | score |
| Pancreatic | 9.1 | ++ | 36.0 | +++ | 4.6 | + | 5.2 | ++ | 2.0 | 0 | 6.7 | ++ | 3.2 | + | 2.1 | + | +8 |
| adenocarcinoma | | | | | | | | | | | | | | | | | |
| Skin cutaneous | 4.7 | + | 0.3 | − | 3.2 | + | 4.7 | + | 1.9 | 0 | 8.8 | ++ | 5.7 | ++ | 4.3 | + | +8 |
| melanoma | | | | | | | | | | | | | | | | | |
| Diffuse large B cell | 11.6 | +++ | n/c | + | 1.0 | 0 | 1.1 | 0 | 3.1 | + | 0.2 | − | 12.9 | +++ | 0.9 | 0 | +6 |
| lymphoma | | | | | | | | | | | | | | | | | |
| Acute myeloid | 1.0 | 0 | n/c | + | 6.4 | ++ | 0.4 | − | 1.5 | 0 | 3.9 | + | 2.1 | + | 357.8 | +++ | +5 |
| leukemia | | | | | | | | | | | | | | | | | |
| Thymoma | 2.2 | + | n/c | + | 0.5 | − | 0.7 | 0 | 7.0 | ++ | 0.2 | − | 7.4 | ++ | 3.8 | + | +5 |

Additionally, ST6Gal1 was upregulated in ovarian serous cystadenocarcinoma, several gastrointestinal tumors (esophageal, gastric, colon and rectal carcinomas, and two brain cancer types (lower grade glioma and glioblastoma). These tumor types together with chromophobe kidney cancer and squamous cell carcinoma of the head and neck with an overall favorable sialyltransferase expression profile without ST6Gal1 contribution emerged as second tier tumor types in this analysis (Table 5).

TABLE 5
Indications with at least 2-fold differential expression of
ST6GAL1 (catalyzes a2,6 sialic acid linkage, preferentially
used by human influenza viruses) in tumors as compared to
their paired normal tissues (FC, fold change). All changes
included in this table are statistically significant.

| | |
|---|---|
| Tumor types with high potential for | ST6GAL1 |

| local infectivity with CodaLytic | FC | Score |
|---|---|---|
| Testicular germ cell tumors | 22.245 | +++ |
| Diffuse large B cell lymphoma | 11.566 | +++ |
| Pancreatic adenocarcinoma | 9.106 | ++ |
| Ovarian serous cystadenocarcinoma | 7.687 | ++ |
| Skin cutaneous melanoma | 4.735 | + |
| Rectum adenocarcinoma | 4.572 | + |
| Stomach adenocarcinoma | 4.572 | + |
| Colon adenocarcinoma | 3.318 | + |
| Esophageal carcinoma | 2.998 | + |
| Low grade glioma | 2.925 | + |
| Glioblastoma multiforme | 2.910 | + |
| Thymoma | 2.221 | + |

Increased presence of α2,3-linked and α2,6-linked sialic acids as well as increased expression of specific sialyltransferases have been previously observed in various tumor types in independent cohorts. In these studies, the functional consequences include both modulation of tumor-intrinsic properties, like increased metastatic propensity in breast cancers overexpressing ST3Gal6, and tumor-extrinsic responses to modified sialylation patterns, e.g., the induction of an adaptive anti-tumor immune response to sialylation of T antigen in breast cancers overexpressing ST3Gal1. Of note, the cutoff criteria for specific sialyltransferases in this holistic analysis did not always replicate the overexpression described in published case studies, likely due to different underlying patient cohorts, different methodologies, and different research questions. As such, the TCGA data-based ranking suggested here should be considered as one of many parameters that collectively will support indication selection for CodaLytic moving forward.

Acknowledging that additional enzymes within the glycosylation pathway contribute to surface expose of α2,3-linked and α2,6-linked sialic acids and multiple cell-intrinsic factors impact productive replication of influenza virus in host cells, these data support the further assessment of pancreatic adenocarcinoma and melanoma as tumor types that are treated by various embodiments of the present invention. Additional hematological tumors are also encompassed.

### Example 3

### Sialic Acid Expression in Human Breast Cancer Tissues

### Creation of the Tissue Microarray (TAMA)

To create a TMA, core biopsies from 75 human breast were assembled. These cover the three major subtypes of breast cancer, i.e. triple-negative breast cancer (TNBC, n=20), hormone receptor-positive Her2-negative breast cancer (HR+HER2-BC, n=20) and Her2-positive breast cancer (Her2+BC, n=20), as well as non-malignant control tissues (n=15). Basic parameters of the tissues are summarized in Table 6. Tissues were originally collected as part of normal patient care and patient informed consent was granted for exploratory research before incorporation into the TMA.

TABLE 6
Tissue microarray map and basic characteristics of the tissues included in the staining.
ER, estrogen receptor positivity; PR, progesterone receptor positivity; Her2neu,
Her2 positivity; Ki-67, tumor cell proliferation score; n/a, not applicable.

| Tissue | TMA | | | Grade | ER | PR | Her2neu | Ki-67 |
|---|---|---|---|---|---|---|---|---|
| type | location | Age | Morphology | (1-4) | (0-100) | (0-100) | (0-3) | (0-100) |
| TNBC | 1A | 64 | Ductal | 3 | 0 | 0 | 0 | 63 |
| | 2A | 54 | Ductal | 3 | 0 | 0 | 1 | 71 |
| | 3A | 83 | Ductal | 3 | 0 | 0 | 0 | 28 |
| | 4A | 35 | Ductal | 3 | 0 | 0 | 0 | 81 |
| | 5A | 54 | Ductal | 3 | 0 | 0 | 0 | 33 |
| | 6A | 90 | Ductal | 3 | 0 | 0 | 1 | 75 |
| | 7A | 39 | Ductal | 3 | 0 | 0 | 0 | 79 |
| | 8A | 45 | Ductal | 3 | 0 | 0 | 0 | 74 |
| | 9A | 67 | Ductal | 3 | 0 | 0 | 0 | 26 |
| | 10A | 61 | Ductal | 2 | 0 | 0 | 0 | 24 |
| | 1B | 57 | Ductal | 3 | 0 | 0 | 0 | 58 |
| | 2B | 66 | Ductal | 3 | 0 | 0 | 0 | 73 |
| | 3B | 64 | Ductal | 3 | 0 | 0 | 0 | 50 |
| TNBC | 4B | 49 | Ductal | 3 | 0 | 0 | 0 | 30 |
| | 5B | 69 | Ductal | 3 | 0 | 0 | 1 | 80 |
| | 6B | 43 | Ductal | 3 | 0 | 0 | 0 | 88 |
| | 7B | 55 | Ductal | 3 | 0 | 0 | 1 | 55 |
| | 8B | 64 | Ductal | 3 | 0 | 0 | 3 | 23 |
| | 9B | 60 | Ductal | 3 | 0 | 0 | 0 | 80 |
| | 10B | 49 | Ductal | 3 | 0 | 0 | 0 | 59 |
| HR + | 1C | 48 | Ductal | 3 | 91 | 0 | 0 | 45 |
| Her2-BC | 2C | 85 | Ductal | 3 | 99 | 0 | 0 | 8 |
| | 3C | 83 | Ductal | 3 | 98 | 81 | 0 | 50 |
| | 4C | 59 | Ductal | 3 | 2 | 0 | 0 | 85 |
| | 5C | 76 | Ductal | 3 | 99 | 0 | 1 | 43 |
| | 6C | 79 | Ductal | 2 | 92 | 89 | 0 | 18 |
| | 7C | 81 | Lobular | 2 | 82 | 0 | 0 | 4 |
| | 8C | 56 | Ductal | 3 | 92 | 89 | 1 | 64 |
| | 9C | 74 | Ductal | 2 | 68 | 0 | 1 | 22 |
| | 10C | 77 | Lobular | 3 | 92 | 3 | 1 | 15 |
| | 1D | 71 | Ductal | 3 | 100 | 99 | 0 | 35 |
| | 2D | 77 | Ductal | 3 | 97 | 2 | 1 | 23 |
| | 3D | 55 | Ductal | 3 | 99 | 0 | 1 | 32 |
| | 4D | 63 | Ductal | 3 | 99 | 0 | 1 | 36 |
| | 5D | 43 | Ductal | 3 | 55 | 0 | 1 | 16 |
| | 6D | 69 | Ductal | 3 | 99 | 86 | 0 | 39 |
| | 7D | 77 | Ductal | 3 | 99 | 91 | 1 | 15 |
| | 8D | 53 | Ductal | 3 | 89 | 91 | 1 | 38 |
| | 9D | 40 | Ductal | 3 | 87 | 60 | 1 | 19 |
| | 10D | 80 | Ductal | 3 | 99 | 92 | 1 | 21 |
| Her2 + | 1E | 69 | Ductal | 3 | 0 | 0 | 3 | 32 |
| BC | 2E | 61 | Ductal | 3 | 0 | 2 | 3 | 15 |
| | 3E | 47 | Ductal | 3 | 90 | 64 | 3 | 31 |
| | 4E | 41 | Ductal | 3 | 41 | 54 | 3 | 55 |
| | 5E | 87 | Ductal | 2 | 96 | 0 | 3 | 17 |
| | 6E | 80 | Ductal | 2 | 0 | 0 | 3 | 13 |
| | 7E | 74 | Ductal | 3 | 98 | 0 | 3 | 48 |
| | 8E | 65 | Ductal | 3 | 97 | 0 | 3 | 39 |
| | 9E | 75 | Ductal | 3 | 10 | 0 | 3 | 12 |
| | 10E | 57 | Ductal | 3 | 0 | 0 | 3 | 51 |
| | 1F | 81 | Ductal | 3 | 0 | 0 | 3 | 31 |
| | 2F | 55 | Ductal | 2 | 0 | 0 | 3 | 29 |
| | 3F | 41 | Ductal | 3 | 8 | 0 | 3 | 46 |
| | 4F | 44 | Ductal | 3 | 94 | 75 | 2 | 60 |
| | 5F | 73 | Ductal | 3 | 0 | 0 | 3 | 19 |
| | 6F | 71 | Ductal | 2 | 0 | 0 | 3 | 29 |
| | 7F | 62 | Ductal | 2 | 98 | 0 | 3 | 23 |
| | 8F | 65 | Lobular | 2 | 98 | 69 | 3 | 42 |
| | 9F | 87 | Ductal | 3 | 0 | 0 | 3 | 38 |
| | 10F | 41 | Ductal | 3 | 8 | 0 | 3 | 46 |
| Non- | 1G | 29 | Fibroadenoma | n/a | n/a | n/a | n/a | n/a |
| malignant | 2G | 31 | Usual hyperplasia | n/a | n/a | n/a | n/a | n/a |
| | 3G | 29 | Fibroadenoma | n/a | n/a | n/a | n/a | n/a |
| | 4G | 62 | Usual hyperplasia | n/a | n/a | n/a | n/a | n/a |
| | 5G | 63 | Intraductal | n/a | n/a | n/a | n/a | n/a |
| | | | papilloma | | | | | |
| | 6G | 47 | Fibroadenoma | n/a | n/a | n/a | n/a | n/a |
| | 7G | 47 | Sclerosing | n/a | n/a | n/a | n/a | n/a |
| | | | adenosis | | | | | |
| | 8G | 40 | Usual hyperplasia | n/a | n/a | n/a | n/a | n/a |
| | 9G | 44 | Usual | n/a | n/a | n/a | n/a | n/a |
| | | | hyperplasia | | | | | |
| | 10G | 83 | Benign | n/a | n/a | n/a | n/a | n/a |
| | | | myofibroblastoma | | | | | |
| | 1H | 54 | Usual hyperplasia | n/a | n/a | n/a | n/a | n/a |
| | 2H | 30 | Usual | n/a | n/a | n/a | n/a | n/a |
| | | | hyperplasia | | | | | |
| | 3H | 57 | Fibrocystic | n/a | n/a | n/a | n/a | n/a |
| | | | changes | | | | | |
| | 4H | 55 | Intraductal | n/a | n/a | n/a | n/a | n/a |
| | | | papilloma | | | | | |
| | 5H | 45 | Atypical | n/a | n/a | n/a | n/a | n/a |
| | | | hyperplasia | | | | | |

### Immunohistochemistry

For tissue staining of α2,3 and α2,6-linked sialic acids, well-established reagents were chosen: Maackia amurensis Lectin II (MAL2, VectorLabs, cat. no. B-1265-1) and Sambucus nigra lectin (SNA, VectorLabs, cat. no. B-1305-2), respectively. Both lectins are directly biotinylated.

### Semi-Quantitative Scoring and Analysis

Individual cores of the TMA were analyzed by an ABP-certified pathologist. Staining intensity in breast cancer nodules or mammary ducts were assigned scores between 0 and 3. In addition, the contribution of tumor tissue, lymphocytic infiltrate and stoma were estimated.

Scores were then graphed for visualization using GraphPad Prism v9.1.2. Cores that were collected from cancer patients but did not contain any tumor cells were excluded from this analysis. Pathologist scores of 0-1, 1-2 or 2-3 were converted to the values 0.5, 1.5 and 2.5, respectively.

Staining intensities were statistically compared across the four different tissue groups using Kruskal-Wallis tests with Dunn's multiple comparisons test, calculated with GraphPad Prism v9.1.2. This non-parametric analysis was chosen based on the assumption of non-normal distributions of human parameters at small sample sizes.

### Results

Human influenza viruses, like the H1N1 influenza A virus CodaLytic is derived from, preferentially use α2,6-linked sialic acids for attachment and entry. This specific linkage is recognized by Sambucus nigra lectin (SNA).

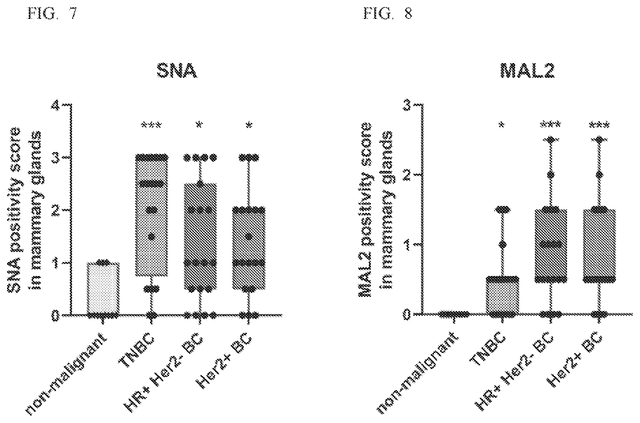

In tissues collected from cancer patients, tumor cells across all subtypes stained mildly to moderately with SNA, while staining in non-malignant mammary ducts was primarily absent (FIG. 7). The differences in SNA staining between tumor and non-malignant ducts were statistically significant for all three subtypes (p=0.0002 for TNBC, p=0.0496 for HR+Her2-BC, and p=0.0373 for Her2+BC vs non-malignant tissues). A trend toward highest staining in TNBC samples was observed.

In addition to α2,6-linked sialic acids, human influenza viruses can also attach to target cells via α2,3-linked sialic acids, albeit less preferentially. Therefore, the same tissues were stained with Maackia amurensis Lectin II (MAL2), that recognizes α2,3-linked sialic acids.

Overall, weaker staining intensity was observed for MAL2 than for SNA (FIG. 8). Staining was completely absent in mammary ducts of all non-malignant tissues analyzed. Weak to mild staining was observed in tumor tissues across subtypes demonstrating statistically significantly higher MAL2 scores as compared to non-malignant specimens (p=0.0274 for TNBC, p=0.0003 for HR+Her2-BC, and p=0.0009 for Her2+BC vs non-malignant tissues). The highest MAL2 scores occurred in HR+Her2-BC and Her2+BC tissues.

### Summary and Conclusions

The immunohistochemical analysis of specific glycosylation patterns recognized by human influenza viruses for attachment and entry into host cells demonstrated increased expression in breast cancer tissues as compared to non-malignant mammary ducts. This hypersialylation is in line with published literature describing the pro-tumorigenic roles of sialic acid and sialyl transferase overexpression in breast cancer.

In this TMA covering grade 2 and 3 breast cancer tissues of different subtypes, trends toward highest α2,6-linked sialic acid expression were observed in TNBC, while α2,3-linked sialic acid sialic acid trended to be highest in HR+Her2− BC, although these differences were not statistically significant. Sample sizes for grade 2 cancers as well as cancers with lobular morphology were too small in this cohort to assess any potential associations with these parameters.

The relative overexpression of influenza attachment and entry receptors on breast cancer tissues represents one mechanism by with virotherapeutic influenza virus specifically targets cancer cells over non-malignant mammary epithelium. This preferential tropism may be particularly exploited by influenza virus A/CA07/09-(HA-NA)^{Min} that exposes fewer HA molecules on the surface of the virus particle as a result of codon-pair deoptimization of the HA gene. Following viral entry, additional differences between tumor and normal cells, such as defective interferon signaling and relative resistance to apoptosis induction, may further contribute to tumor cell specificity of the virus.

### Example 4

### Sialic Acid Expression and Infectivity Assessment of Different Cell Lines by Flow Cytometry

### Cells and Media

Cell lines: HCC1937 human TNBC cell line (ATCC CRL-2336); MDA-MB-231 human TNBC cell line (ATCC CRM-HTB-26); EMT6 murine TNBC cell line (ATCC CRL-2755); MDCK canine kidney cell line (ATCC CCL-34)
Cell media: RPMI (Gibco, cat. no. 11875-093), supplemented with 10% fetal bovine serum (FBS, Gibco, cat. no. 10082147, lot 1982167), used for HCC1937 and MDA-MB-231; Waymouth MB 752/1 (Millipore Sigma, cat. no. W1625), supplemented with supplemented with 15% fetal bovine serum, used for EMT6; DMEM (Gibco, cat. no.11965-084, supplemented with 10% FBS for MDCK

Cells were seeded in 6-well plates at 3×10⁵ cells/well in 2 mL of their respective medium. 3 wells were seeded for each cell line: one to serve as uninfected controls and two for the 12 h infection time point. Cells were incubated at 37° C., 5% humidity overnight until infection.

### Infection

For infection, media was removed from the cells, cells were washed once with PBS (Gibco, cat. no. 14190-136) and then infected by adding 500 μL of virus working stock or control infection medium to each well. Cells were incubated for 30 min at room temperature, 500 μL of infection media were added per well and cells were incubated for an additional 1 h at 37° C. before the virus inoculate was removed. Cells were incubated in 2 mL of their respective cell culture medium.
Virus stock: Lot E2669/6/6 1-1028119-1, 2×10¹⁰ PFU/ml
Virus stock dilution: 1:33,333.3 in OptiPRO SFM (Gibco, cat. no. 12309019) supplemented with 0.2% bovine serum albumin (Lampire Biological Laboratories, cat. no.7500812), resulting in a 6×105 PFU/mL working stock
Control: OptiPRO SFM, supplemented with 0.2% serum albumin

### Staining and Flow Cytometry

12 h post infection, cells were harvested using 500 uL TrypLE Express (Gibco, cat. no. 12605-010) until cells detach. The TrypLE reaction was stopped using 2 mL of cell culture media and cells were washed three times by centrifugation at 2,000 rpm for 5 min and resuspension of the pellet in 1 mL of ice-cold PBS. After the final wash step, cells were resuspended in 300 μL of FACS buffer (0.5% bovine serum albumin (Lampire Biological Laboratories, cat. #7500812) in PBS with 0.05% sodium azide (Sigma, cat. no. S2002), stored at 4° C. away from light). Primary staining reagents for surface marker were added according to the table below and incubated in the dark for 45 min at 4° C. At this and following steps, single stain and an unstained control with uninfected control cells were included.

| | | | Volume used | |
|---|---|---|---|---|
| Reagent | Vendor and lot | Concentration | per sample | Target |
| Maackia Amurensis | Vector | 660 ug/mL in | 5 uL | α2,3-sialic acids |
| Lectin I (MAL I)- | Laboratories, | PBS | | (Siaα2-3Galβ1- |
| FITC | FL-1311-2 | | | 4GlcNAc) |
| Sambucus Nigra | Vector | 100 ug/mL in | 10 uL | α2,6-sialic acids |
| Lectin (SNA)- | Laboratories, | PBS | | (Siaα2-6Galβ1- |
| Cy5 | CL-1305-1 | | | 4GlcNAc) |
| Mouse anti-HA | Codagenix pool, lot | n/d | 2 uL | Hemagglutinin of |
| A/California/04/09 | no. 1-092216-03-23 | | (1:150) | human influenza |
| neutralizing | (preparation protocol | | | virus, strain |
| antibody | in Appendix 1) | | | A/California/04/09 |

Following incubation, cells were washed three times by centrifugation at 2,000 rpm for 5 min and resuspension of the pellet in 1 mL of ice-cold FACS buffer. After the final wash step, cells were resuspended in 300 μL of FACS buffer containing 0.2 ug/mL PE-conjugated goat anti-mouse IgG secondary antibody (BioLegend, cat. no. 405307) and cells were incubated in the dark for 45 min at 4° C.

Following secondary staining, cells were washed three times by centrifugation at 2,000 rpm for 5 min and resuspension of the pellet in 1 mL of ice-cold FACS buffer. After the final wash step, cells were fixed by resuspension in 300 μL of Fixing Solution (0.5% paraformaldehyde (PFA) in FACS buffer, generated by diluting a 10% neutral buffered formalin stock solution containing 4% total PFA (TissuePro, cat. no. NBF-03032R) 1:8 in FACS buffer) and cells were stored in darkness at 4° C. util analysis by flow cytometry.

Samples were acquired using a BD FacsCaliburBD.CellQuest software v3.3.

### Gating and Analysis

Flow cytometry data was analyzed using BD.CellQuest software v3.3. The population of single cell excluding debris and aggregates was identified using forward and side scatter plot and further analyzed for the presence of specific markers using histogram plots. Unstained and single-stained controls for each cell line were used for setting of marker-specific gates and for the compensation purposes. Frequencies of cells positive for each marker were exported and further analyzed using GraphPad Prism v9.1.0. For infected samples, duplicates were averaged using means. Fold changes of frequencies in infected samples over uninfected control samples were calculated when comparing means and statistically significant differences within each cell line was calculated using ordinary two-way ANOVA with Sidak's multiple comparisons test.

### Results

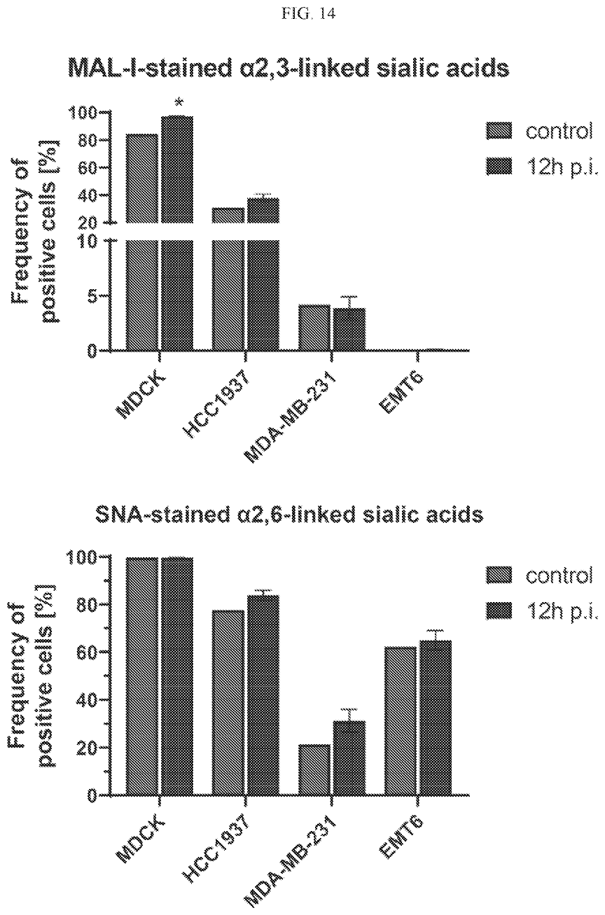

Human and mouse TNBC cell lines displayed a differential pattern of α2,3- and α2,6-linked sialic acids on their cell surface (FIG. 14). Surface expression of α2,3-linked sialic acids was moderate in comparison to >80% positivity in MDCK cells, often used for production of influenza viruses. Both human cell lines had stronger α2,3 sialylation than the mouse cell line EMT6 (31% for HCC1937 and 4% for MDA-MB-231), which was less than 1% positive by MAL-I staining. In contrast to that, α2,6 sialylation was overall more frequent in all TNBC cell lines (range 28% in MDA-MB-231 to 85% in HCC1937), including >60% positivity in EMT6. Infection of the cells with influenza virus A/CA07/09-(HA-NA)^{Min} generally did not change sialylation frequency with none of the fold changes above 1.5-fold as compared to the matching uninfected control. Only the increase of MAL-I staining positivity in MDCK cells from 85% to 97% (1.15-fold) reached statistical significance (p=0.0116).

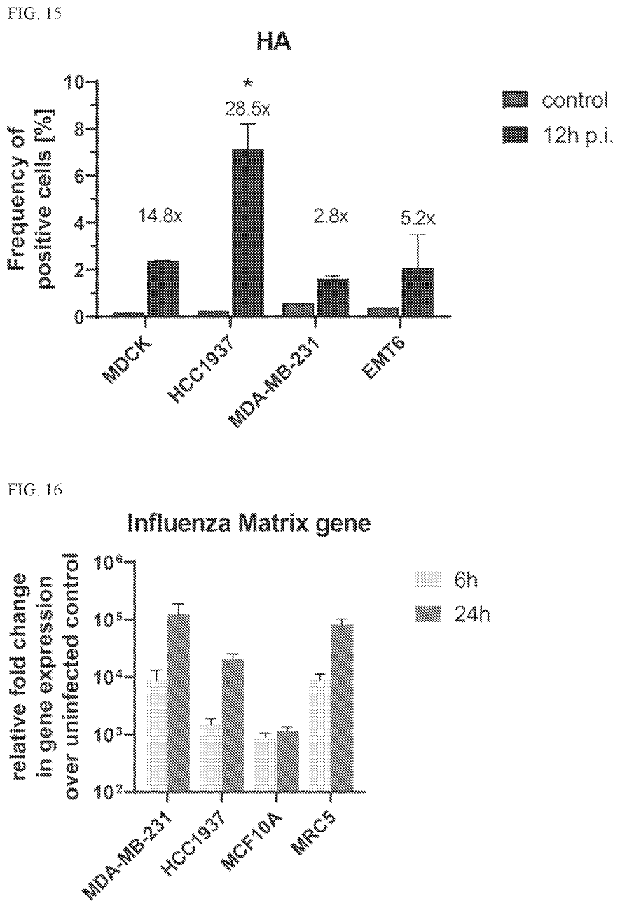

To assess how sialic acid surface exposure related to ability of influenza virus A/CA07/09-(HA-NA)^{Min} to infect the different cell lines, HA surface expression as an indicator of late stage viral infection was quantified (FIG. 15). All four cell lines showed an increase in HA surface expression after 12 h of infection with 3×10⁵ PFU/well, although infectivity rates were overall low in all cells under these conditions, including in MDCK cells (range 1.5% HA positivity in MDA-MB-231 cells to 7.9% in HCC1937). MDA-MB-231 cells showed the lowest fold change over analogously gated non-infected cells (2.8-fold), HCC1937 were most effectively infected with a 28.5-fold increase in HA expression. The murine cell line EMT6 was infected to a degree within the range of the human MDA-MB-231 TNBC cells and the production cell line MDCK.

### Summary and Conclusions

In this experiment, sialylation patters in various human and mouse TNBC cell lines were characterized and infectivity with influenza virus A/CA07/09-(HA-NA)^{Min} was studied in vitro. α2,3-linked and α2,6-linked sialic acids were expressed to varying degrees on these cell lines and in all cases to a lesser extent that on MDCK cells, often used for propagation of influenza viruses. Human TNBC cell lines expressed both sialic acid forms, while the murine cell line EMT6 only expressed α2,6-linked sialic acids. All cell lines could be infected with A/CA07/09-(HA-NA)^{Min} as determined by surface expression of the late protein HA, albeit at low rates. HCC1937 cells showed the highest infection rate, in line with highest presence of both sialic acid forms. Both EMT6 and MDA-MB-231 cells expressed similar degrees of HA on their surface.

In contrast to avian influenza viruses, human influenza viruses prefer α2,6 sialic acid linkage and infectivity is correlated with this preference. The observation that ranking of infection efficiency matched better with SNA staining of α2,6-linked sialic acids is in line with this literature. However, other factors may contribute to infectivity and production of HA protein in different cell lines.

### Example 5

### Induction of Immunostimulatory Genes in TNBC Cell Lines Infected With Influenza Virus A/CA07/09-(HA-NA)^{Min}

### Experimental Overview

Human triple-negative breast cancer (TNBC) cell lines HCC1937 and MDA-MB-231 as well as control cell lines MCF10A (normal human breast cells) and MRC5 (normal human lung fibroblasts) were plated in monolayer culture and infected with 3×10⁶ PFU of influenza virus A/CA07/09-(HA-NA)^{Min}. Cells were harvested at 6 h and 24 h post infection, their RNA was isolated and reverse transcribed to cDNA. Quantitative polymerase chain reaction (qPCR) was subsequently performed to evaluate expression of genes responsible for activation of the immune system.

### Results

Successful infection of the different cell lines used in this study were confirmed by analyzing gene expression of the influenza matrix gene M. Matrix mRNA was detectable in all cell lines after infection with influenza virus A/CA07/09-(HA-NA)^{Min} and the relative induction increased over time in the breast cancer cell lines MDA-MB-231 and HCC1973 as well as in the fibroblast cell line MRC5 (FIG. 16). This increase over time suggests active infection in these cell lines. Of note, this was not observed in non-tumorigenic mammary epithelial cells MCF10A, indicating decreased infectivity. MRC5 have been described in the past as a cell culture system to detect suspected influenza virus infection in pharyngeal swabs with similar sensitivity to commonly used Madin-Darby canine kidney (MDCK) cells. In this context, MRC5 can be considered as positive control for infection.

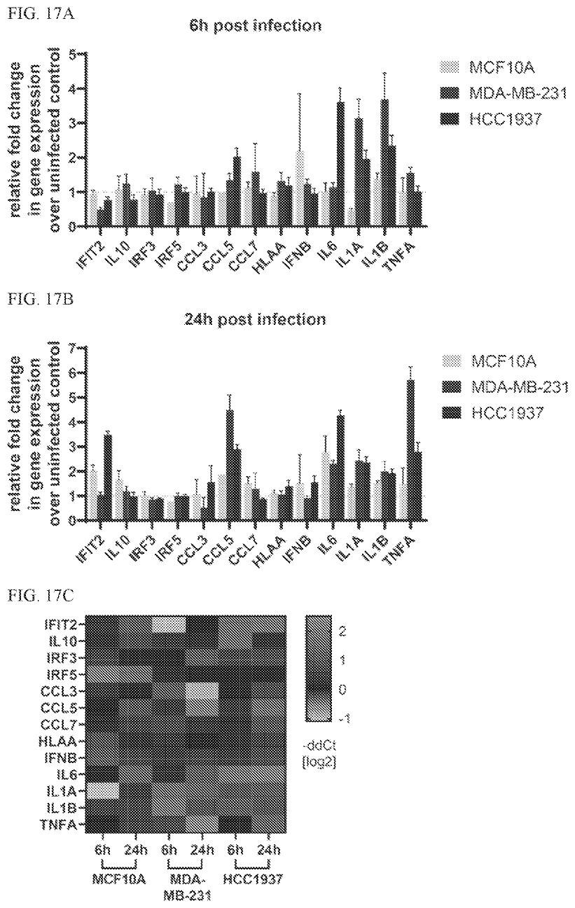

Gene expression changes in the cell lines of breast origin are summarized in FIG. 17. In both tumor cells lines, a consistent increase in expression of the antiviral genes interleukin 1 alpha (3.13-fold and 2.43-fold in MDA-MB-231 and 1.97-fold and 2.34-fold in HCC1397 at 6 h and 24 h, respectively) and interleukin 1 beta (3.70-fold and 1.99-fold in MDA-MB-231 and 2.34-fold and 1.92-fold in HCC1397 at 6 h and 24 h, respectively) at both time points was observed, that was absent in the non-tumorigenic mammary epithelial cells MCF10A.

Induction of the antiviral factor TNFalpha was observed only in the two tumor cell lines at 24 h (5.70-fold in MDB-MB-231 and 2.78-fold in HCC1937), but not in MCF10A (1.48-fold over uninfected controls), aligned with the increase in influenza M gene expression in comparison to the 6 h time point. This observation is in accordance with literature describing TNFa being induced after influenza infection.

The chemoattractant CCL5 was more strongly induced in the two cancer cell lines at 24 h (4.49-fold in MDB-MB-231 and 2.89-fold in HCC1937) than in MCF10A (1.85-fold over uninfected controls).

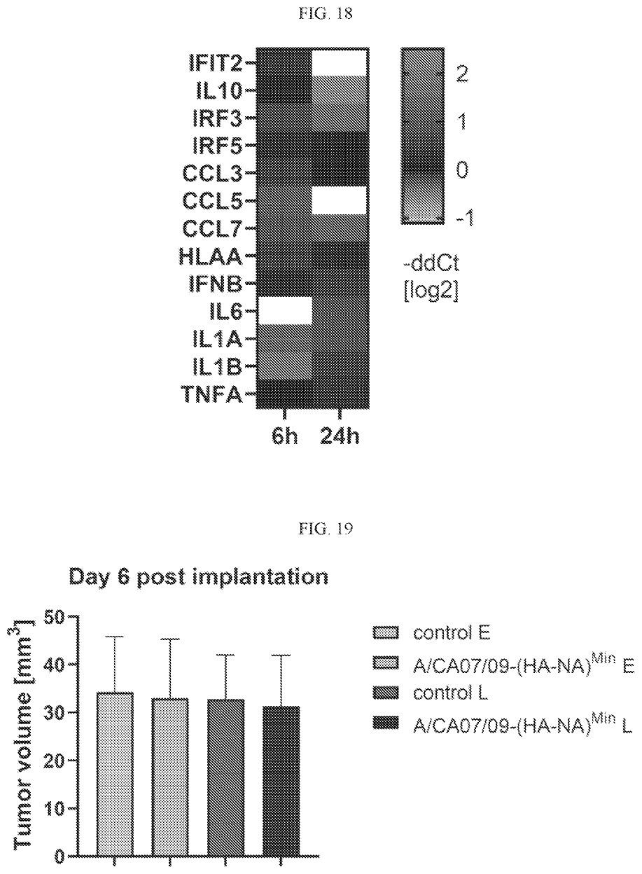

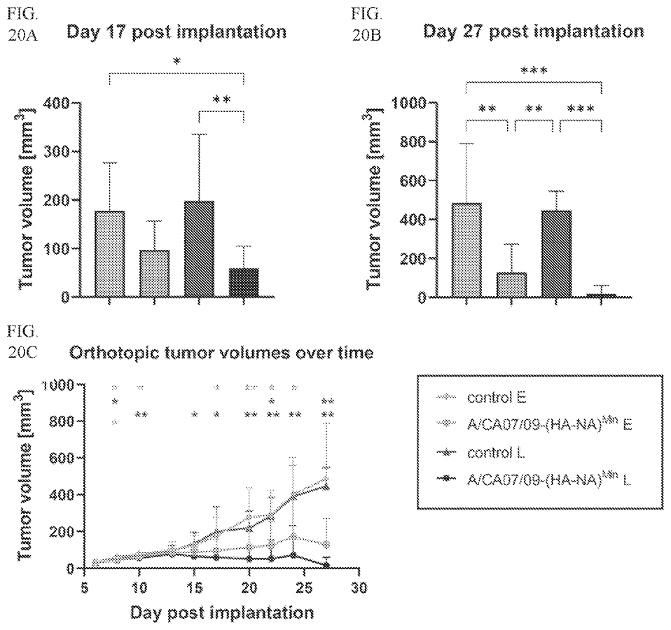

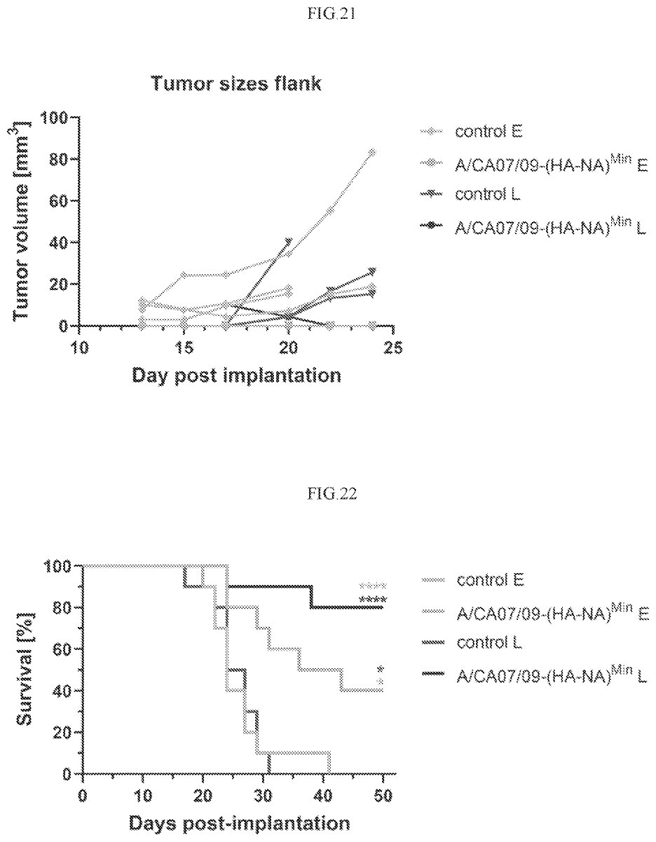

For reference, gene expression changes in the MRC5 fibroblasts are shown in FIG. 3 at the same color scale as for the breast cell lines. Overall, gene expression changes in either direction were less pronounced, although IL1a and IL1b were similarly induced at 6 h post infection than in breast cancer cell lines (FIGS. 18 and 17C). There were three outliers in gene expression change for MCF5, with −ddCt values of 2.55 for IL6 at 6 h, 4.61 for IFIT2 at 24 h and 8.854 for CCL5 at 24 h post infection. This extreme induction of expression required further confirmation.

### Summary and Conclusions

In this study, the impact of acute infection with influenza virus A/CA07/09-(HA-NA)^{Min} on expression of gene implicated in antiviral response was characterized in vitro over time in breast cancer and comparator cell lines. Overall, the magnitude of expression changes was modest both at 6 h and 24 h post infection. However, in both breast cancer cell lines the antiviral response genes TNFa, IL1a and IL1b and the chemoattractant CCL5 were induced most meaningfully. Of note, this induction was not observed in MCF10A non-tumorigenic mammary epithelial cells, that were not productively infected based on influenza matrix gene expression, suggesting causality of gene expression changes.

Taken together, this data provides evidence for both preferential infection of breast cancer cells over non-transformed mammary epithelium and the induction of proinflammatory genes in response to this infection. The same cytokines are associated with anti-tumor immune responses, which are expected to be induced as a secondary effect in vivo and contribute to the mechanisms of action of influenza virus A/CA07/09-(HA-NA)^{Min}, the virus contained in the CodaLytic drug product.

### Example 6

### qPCR Analysis of Influenza Virus A/CA07/09-(HA-NA)^{Min} Treated ENMT6 Tumors

### Mice

Animal model: Mus musculus; Mouse Strain: Balb/C (Taconic); Age: 8-9 weeks old (Female); IACUC protocol: 2019-01-17-COD-1; Group size: 5 Cells and media
Cell line: EMT6 mouse triple-negative breast cancer (TNBC) cell line (ATCC, CRL-2755); Cell growth medium: Waymouth MB 752/1(Millipore Sigma, cat. no. W1625, lot no. SLCC1320) supplemented with 15% heat0 inactivated fetal bovine serum (FBS, Gibco, cat. no. 10-082-147, lot no. 1982167); Cell concentration: 1×10^{5/50} uL (passage 5)

EMT6 cells were cultured at 37° C., 5% humidity and harvested using TrypLE Express (Gibco, cat. no. 12605-010) until cells detached from the culture flask. The trypsin reaction was stopped using Waymouth culture media with 15% FBS (see above). Cells were washed twice in phosphate buffered saline (PBS, Gibco, cat. no.14190-136), counted using a hemocytometer and Trypan Blue (Gibco, cat. no. 15250061) and cell viability was over 98%. EMT6 cells were centrifuged at 2,000 rpm for 5 min and resuspended to a concentration of 1×10⁵/50 uL in serum free Waymouth medium.

### Tumor Implantation and Treatment

Balb/C mice were anesthetized with 50 μL of a mixture of 50 mg/mL ketamine (Sigma, cat. no. K2753) and 5 mg/mL xylazine (Sigma, cat. no. X1251) injected intraperitoneally as approved by the Institutional Animal Care and Use Committee (IACUC), shaved around inguinal mammary fat pads and injected orthotopically with 50 μL of tumor cell suspension into the left fat pad. Animals were monitored daily for palpable tumors. On day 6 post implantation, tumors were palpable in all mice. Mice were assigned to different treatment groups and injected intratumorally with 50 μL of PBS or influenza virus A/CA07/09-(HA-NA)^{Min}.
Virus stock: Lot 3-120820-2 (1-8), 3×1010 PFU/ml Virus stock dilution: 15× in PBS (Gibco, cat. no.14190-136); Control: PBS

Intratumoral injections were repeated on days 8, 10, 13 and 15 post implantation and tumor volumes were measured three times a week. Tumor growth was monitored by measurements of three perpendicular axes (A, B, C) with calipers and the volume was calculated using the following formula: V=0.52*ABC (ellipsoid volume).

### Tumor Processing

On day 16, mice were sacrificed, tumors were removed and stored on ice in 10 mL of RPMI (Gibco, cat. no. 11875-093), supplemented with 2% FBS. Next, tumors were mechanically dissociated using a magnetic-activated cell sorting (MACS) dissociator (Miltenyi, program name Mouse ImpTumor_04_01) and the homogenates were frozen at −80° C.

### RNA Isolation and Reverse Transcription

For RNA isolation, Qiagen Viral RNA Isolation Kit (Qiagen, cat. no. 52906) was used according to the manufacturer's protocol. 140 μL of each tumor homogenate were used per purification. RNA was eluted from the column using 2×40 μL of dH₂O, checked for concentration with NanoDrop.

Reverse transcription was performed using High-Capacity cDNA Reverse Transcription Kit (Thermo Fisher, cat. no. 4368813) without RNAse inhibitor according to manufacturer's instructions. 150 ng of freshly isolated RNA were used as template. Remaining RNA was frozen at −80° C.

Quantitative Polymerase Chain Reaction (qPCR)

qPCR was performed using QuantStudio3 machine from Applied Biosystems at default ddCt setting and 2× SYBRGreen PCR Master Mix (Applied Biosystems, cat. no. 4309155, lot no.2102530). As a template, 4 μL of cDNA were used at 1:10 dilution. Every reaction was performed in 10 μL of volume (1 uL of 5 mM primer mixture, 4 μL of template and 5 μL of 2× SYBRGREEN mix). Each sample was run in duplicate.

Expression of the following genes was analyzed:

| GAPDH | Glyceraldehyde 3-phosphate dehydrogenase |
|---|---|
| IL1A | Interleukin 1 alpha |
| IL1B | Interleukin 1 beta |
| IL15 | Interleukin 15 |
| IFNa | Interferon alpha |
| IFNb | Interferon beta |
| IL18 | Interleukin 18 |
| IRF3 | Interferon regulatory factor 3 |
| IRF5 | Interferon regulatory factor 5 |
| IFIT2 | Interferon-induced protein with tetratricopeptide repeats 2 |
| CCL3 | C-C motif chemokine ligand 3 |
| CCL5 | C-C motif chemokine ligand 5 |
| CCL7 | C-C motif chemokine ligand 7 |
| CCR7 | C-C motif chemokine receptor 7 |
| TNFa | Tumor necrosis factor alpha |
| IFNg | Interferon gamma |
| GrzB | Granzyme B |
| MHCII | Major histocompatibility complex II |
| CD86 | Cluster of differentiation 86 |
| CD40L | Cluster of differentiation 40 ligand |
| IL12A | Interleukin 12 alpha |
| PD-L1 | Programmed death ligand 1 |
| TGFb | Transforming growth factor beta |
| IDO | Indoleamine 2,3-dioxygenase 1 |

Sequences of the primer pairs were taken from the Origene website at www.origene.com/category/gene-expression/qpcr-primer-pairs, which contains set of verified and validated qPCR primers for mouse and human genes.

Samples were run using default AACT protocol on QuantAmp3 qPCR machine (firmware version 1.5.1) with 10 μL of total volume.

### Data Analysis

Tumor volumes from each experimental group at each day of measurement were averaged using mean and standard error of mean. Differences in tumor growth over time was assessed using RM two-way ANOVA with Sidak's multiple comparisons test calculated in GraphPad Prism v9.1.0.

qPCR data were included in the analysis if at least 3 of 5 samples in each group returned Ct values below 35. Otherwise, data were not considered reliable and such gene was not taken under further analysis in results section. Ct values for each duplicate measure were averaged and then were normalized against for the mean GAPDH Ct value in the matching animal (dCt) using Microsoft Excel (Windows 10 Business v20H2), multiplied by −1 for better visualization and plotted for each animal using GraphPad Prism v9.1.0. Differences in gene expression were determined using two-tailed t-tests.

### Results

Intratumoral treatment with influenza virus A/CA07/09-(HA-NA)M resulted in statistically significant decrease in tumor size on day 15 (p<0.001, FIG. 24).

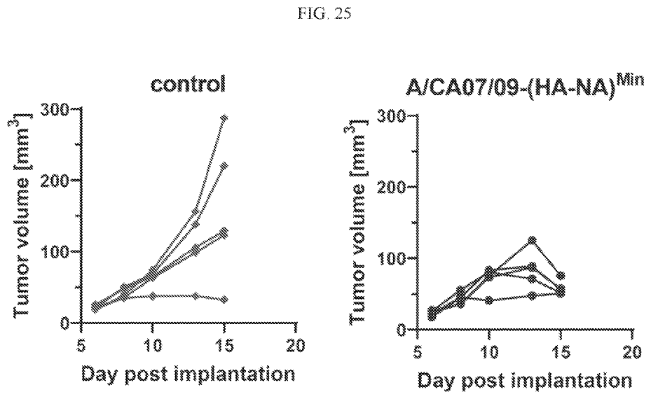

All virus-treated tumors showed shrinkage during the course of treatment (FIG. 25). In the control group, the rate of tumor growth varied, including one tumor (animal M2) that did not continue to grow beyond day 8.

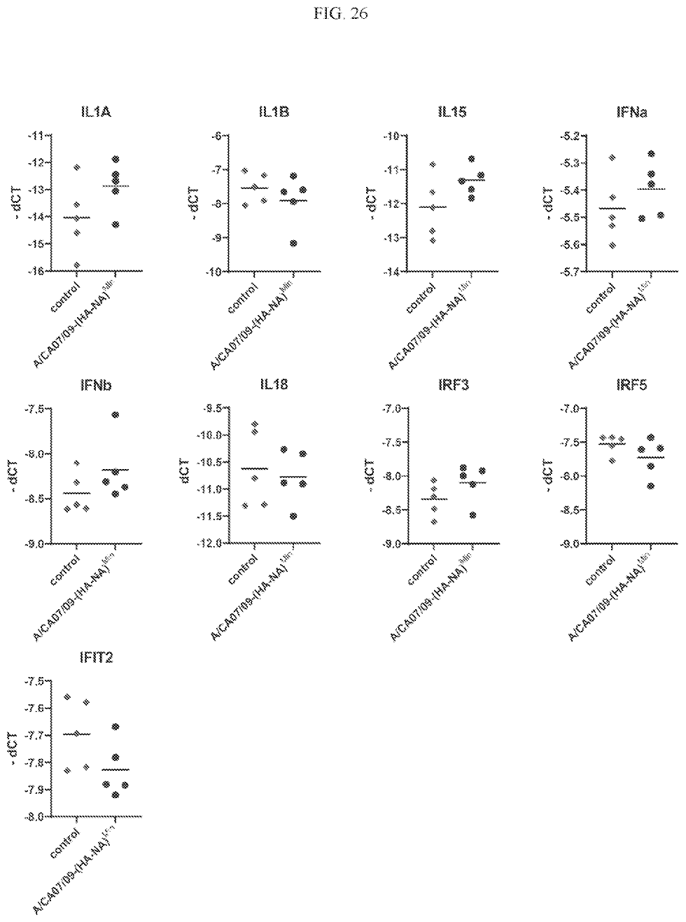

Gene expression data is summarized in FIGS. 26-29. Expression of genes commonly associated with an anti-viral innate immune response did not change significantly after treatment with influenza virus A/CA07/09-(HA-NA)^{Min} comparison to control treatment (FIG. 3). However, there were trends toward increased expression of IL1A and IL15.

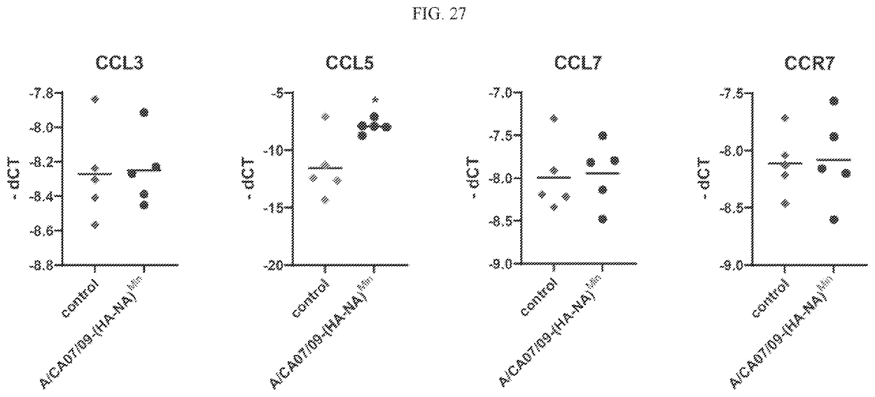

Expression of chemokine and chemokine receptor genes are shown in FIG. 27. Virus treatment significantly increased the expression of CCL5 (p=0.02), which serves as a chemoattractant for T and NK cells. Of note, the animal with the highest CCL5 expression in the control group corresponds to the animal with the smallest tumor volume (C2).

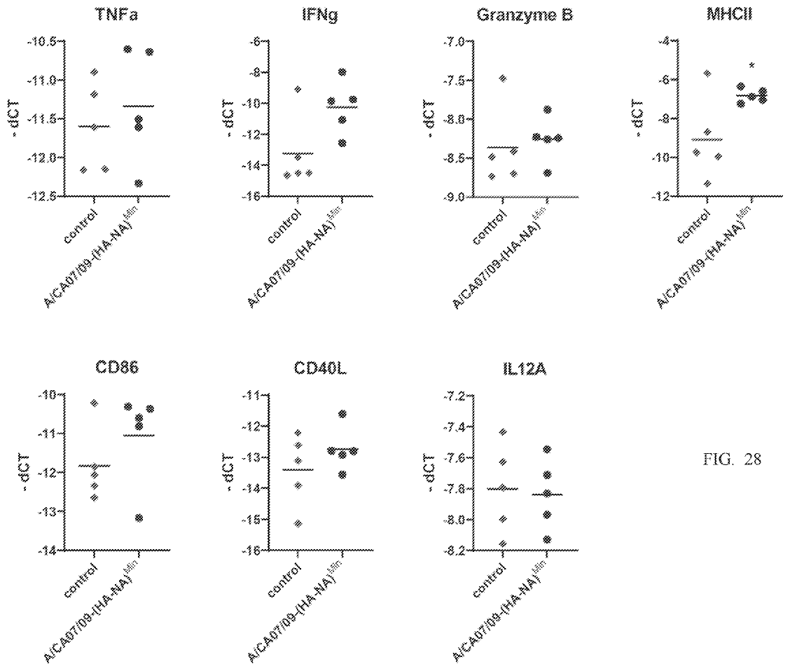

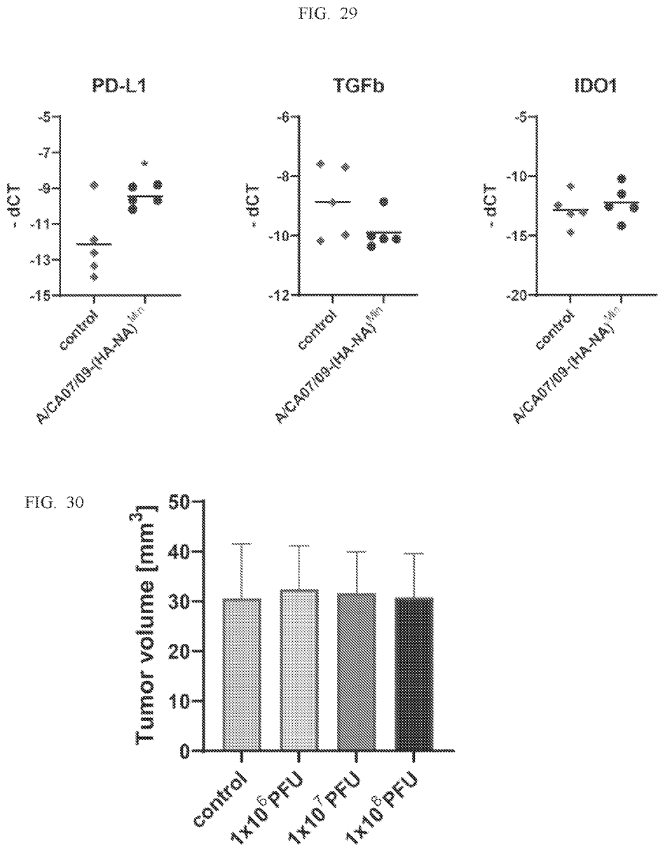

Gene expression for several molecules associated with anti-tumor immunity increased after A/CA07/09-(HA-NA)^{Min} treatment, including the T cell effector cytokine IFNg (p=0.05021), the dendritic cell activation marker CD86 (p=0.29), and the antigen presentation molecule MHC-II (p<0.05, FIG. 28). In all these cases as well as for granzyme B, the animal with the highest expression in the control group corresponded to the animal with the lowest tumor volume.

Gene expression of TGFb and IDO1, two molecules associated with immuno-suppression in the tumor microenvironment, was not significantly altered by CodaLytic treatment at the analyzed time point, although there was a trend toward decreased TGFb expression (p=0.13, FIG. 29). In contrast, PD-L1 expression was significantly induced by virus treatment (p<0.05) with animal C2 again having the highest expression in the control group and similar to the expression levels in the virus-treated group.

### Summary and Conclusions

In this experiment, pharmacodynamic changes in the tumor microenvironment induced by intratumoral treatment with influenza virus A/CA07/09-(HA-NA)^{Min}, the virus contained in the CodaLytic drug product, were characterized. Volumes of orthotopic EMT6 TNBC tumors decreased after virus treatment as intended. Gene expression changes in tumors were analyzed 10 days after onset of treatment, before regressing tumors were eliminated, in order to quantify gene expression in tumor and immune cells in bulk.

Significant changes in gene expression after virus treatment were observed for the chemoattractant CCL5, the antigen presenting molecule MHCII, and the B7 family member PD-L1. While PD-L1 functions as a ligand for the inhibitory immune checkpoint receptor PD-1, its dynamic upregulation upon immunotherapy has been observed in response to immunomodulators including other oncolytic viruses both in animal models and in humans.

The significant induction of MHCII and the trend towards induction of CD86 suggest an increase in antigen presentation, supported by meaningful induction of IFNg expression. In aggregate, the gene expression changes in tumor lysate from A/CA07/09-(HA-NA)^{Min}-treated animals suggest the induction of adaptive immune responses, that likely contribute to anti-tumor efficacy.

### Example 7

### Nanostring Analysis of Gene Expression within

### Influenza Virus A Ca07/09-(Ha-Na)^{Min} Treated EMT6 Tumors

### Experimental Overview

EMT6 murine breast cancer cells were implanted into the mammary fat pads of Balb/C mice. Once palpable, tumors were treated with intratumoral injections of 10 PFU or 10⁷ PFU of Influenza virus A/CA07/09-(HA-NA)^{Min} or L15 control medium. Injections were performed three times a week for a total of 5 doses with 10 PFU or 10⁷ PFU. Additionally, two groups of mice that received only single dose of treatment (L15 MOCK or 10⁸ PFU of virus) 12 h before tumor harvest were included. Tumors were collected on day 16 after implantation, homogenized and RNA was isolated and send for analysis with nCounter® Analysis System by Nanostring Technologies (Seattle, WA).

### Results

### Tumor Growth

Intratumoral treatment with 10⁸ PFU of Influenza virus A/CA07/09-(HA-NA)^{Min} resulted in a significant decrease in tumor size (p<0.001 on day 15 post implantation), while treatment with 10⁷ Pfu dose did not produce significant therapeutic effect (p=0.89). In mice from groups D,E that did not receive treatment till day 15 post implantation tumors displayed similar sizes.

Gene Expression Analysis after 5 Rounds of Treatment

Comparison of samples treated with MOCK vs Influenza virus A/CA07/09-(HA-NA)^{Min} showed that tumors treated with 10⁸ PFU dose displayed changes in strikingly more cellular pathways than those treated with 10⁷ PFU dose.

Since 10⁸ PFU dose showed much stronger effect on gene expression compared and 10⁷ PFU dose and its anti-tumor effect was more pronounced in the further analysis we focused on direct 10⁸ PFU vs MOCK comparison. Analysis of direct heat map specifically showed which pathways were upregulated and which downregulated in the virus treated samples.

The most upregulated pathways were connected to the T cell activity: including TCR signaling, costimulation by CD28 molecule family, and immunoregulatory interactions. Also, pathways related to B cell functions such as signaling by Fc receptor and B cell receptor were clearly activated. Moreover, analysis revealed upregulation of pathways involved in antigen processing and presentation both through MHCI and MHCII. On the other hand treatment with influenza virus A/CA07/09-(HA-NA)^{Min} downregulated signaling through receptor Tyrosine Kinases, TGFb and mTOR pathway as well as inhibited pathways responsible for remodeling of extracellular matrix (ECM).

Next, we looked at the changes in the expression of individual cellular genes. We assumed fold change >1.5 with p value <0.05 to be biologically relevant. Data analysis with nSolver Advanced Analysis software identified 43 such genes while analysis with ROSALIND returned 58 genes.

Analysis revealed upregulation of T cells associated genes such as CD3, CD6, CD28 and CD247. Additionally, genes responsible for cytotoxic activity of CD8+ and NK cells such as granzyme A and B (Gzma, Gzmb) Fas Ligand (Fasl) and Nkg7 were upregulated in the virus treated tumors. Another interesting genes were CCL5 (RANTES) chemokine involved in activation of NK and T cells, H2-Ab1 and H2-T23, parts of MHCI and MHCII antigen presentation complexes, and CD86 activation molecule of DC and T cells. Upregulation of these genes clearly indicated that treatment with Influenza virus A/CA07/09-(HA-NA)^{Min} induces activation of T cells, cytotoxicity and improved antigen presentation, all of these being desired phenomena during cancer treatment

On the other hand, treatment with virus induced also immunosuppressive mechanisms. Genes coding for PD1 receptor and its ligand PDL2 (Pdcd1 and Pdcd1Lg2) were among the most upregulated. Most downregulated genes included receptor for epidermal growth factor (EGFR) and matrix metalloproteinase 9 (MMP9). Both those genes are known to be expressed by cancer cells so their downregulation was in accordance with the observation that treatment with Influenza virus A/CA07/09-(HA-NA)^{Min} induces tumor shrinkage and regression.

Gene Expression Analysis after Single Round of Treatment

Next, we analyzed RNA changes in cellular pathways induced by single treatment with Influenza virus A/CA07/09-(HA-NA)^{Min}.

12 h after single treatment, the most upregulated pathway was the ones correlated to interferon signaling. Such change was expected, since infection with flu was supposed to induce potent interferon response. Interestingly, changes in other pathways looked similar between tumors after single and five rounds of treatment. We saw upregulation of signaling through B and T cell receptors, increased class MHCI mediated antigen processing and downregulation of MTOR activity. However, while the affected pathways were similar rate of change was stronger in tumors that received a single dose of virus.

Analysis of cellular genes that were mostly affected by a single treatment returned 150 genes with significantly changed expression (fold change >1.5 with p value <0.05).

Upregulated genes included the ones related to interferon and anti-viral response such as IFIT3, IFIT2, viperin (Rsad2), and 2-5-Oligoadenylate Synthetase Like protein (OasL1). Other notable hits included genes involved in antigen processing (Tap2, H2-Q2), cytokines and their receptors (IL15, IL1m, IL6ra) chemokines (eotaxin-1, CCL9, CCL11) and NFkb transcription factor. Profile of upregulated genes confirmed that interferon pathway, antigen presentation and proinflammatory cytokine signaling are mostly affected by early infection with Influenza virus A/CA07/09-(HA-NA)^{Min} Downregulation affected several genes from different pathways: among those chemokines CXCL14 and CXCL16, Integrin alpha L(ItgaL, CD11a) and MMP9. Interestingly H2-Aa member of MHCII antigen presentation machinery was also significantly downregulated suggesting that infection with Influenza virus A/CA07/09-(HA-NA)^{Min} may skew antigen presentation towards MIHCI rather than i MHCII context.

### Expression of Influenza Viral Genes

In the final step we analyzed the expression of viral genes in the Influenza virus A/CA07/09-(HA-NA)^{Min} treated samples. In the tumors that received the single treatment 12 h after administration viral RNAs were abundant, with all viral genes being significantly upregulated (FIG. 41A). Interestingly, levels of RNAs for individual genes varied with number of detected molecules for M2 being over 70× higher than for PB1 (FIG. 41B).

Since viral particles contain RNAs at equal stoichiometric ratio, such observation suggested that RNA replication must be taking place in virus treated tumors and some RNAs are amplified more efficiently. RNA containing deoptimized sequences (HA and NA) genes were present at lower levels compared to the wild-type RNAs coding for matrix proteins. For instance, the levels of HA RNA were almost 7.5× lower than the levels of M2 mRNA and 2.5× lower than mRNA for NP and M1 (FIG. 41B). This could suggest decreased stability of deoptimized mRNA molecules and their impaired ability to replicate.

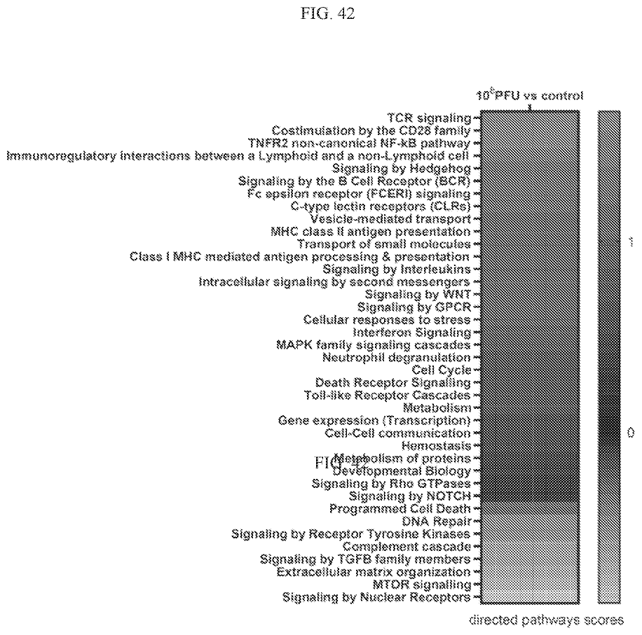

Compared to the samples that were treated only once, tumors that received 5 rounds of treatment showed dramatic decrease in the levels of viral RNAs (FIG. 42).

Moreover, analysis of differential expression after 5 rounds of treatment showed that out of 11 analyzed influenza genes only NS1 showed statistically significant upregulation over the MOCK-treated samples with increase not exceeding 2×. This means that from the statistical point of view viral RNA was practically cleared from the tumors within 12 h of administration. Such result suggest that after 10 days of treatment with Influenza virus A/CA07/09-(HA-NA)^{Min} a potent immune response develops that effectively clears subsequent doses of virus and prevents its replication.

### Conclusions and Discussion

Treatment of EMT6 tumors with Influenza virus A/CA07/09-(HA-NA)^{Min} inhibits tumor growth and leads to disease clearance in ˜50% of animals. While therapy produces consistent results, the exact mechanism by which virus executes its anti-cancer effect remains unknown. Here we performed large scale analysis of RNA isolated from the Influenza virus A/CA07/09-(HA-NA)^{Min} treated tumors and compared changes in gene expression versus MOCK treated samples. Analysis was executed using Nanostring technology including nSolver and ROSALIND software platforms.

In the first step we evaluated changes of gene expression in the animals that were treated with virus for 10 days including 5 rounds of intratumoral treatment. Two groups treated with different doses (10⁸ and 10⁷ Pfu) and a MOCK treated control were analyzed. We found that tumors treated with 10⁸ Pfu show much stronger changes in cellular pathways compared to the ones treated with 10⁷ PFU. Considering that treatment with 10⁸ PFU also produced much stronger therapeutic effect than 10⁷Pfu, we focused on this group in our further analysis. We found that treatment with Influenza virus A/CA07/09-(HA-NA)^{Min} upregulates several pathways mostly related to activation of T cells: signaling through TCR receptor, costimulation by CD28 and interaction between lymphoid and non-lymphoid cells. Such observation was consistent with results of our flow cytometry experiments that detected increased infiltration of T cells in the virus treated samples accompanied by elevated levels of CD86 activation molecule on T lymphocytes. Moreover, treatment with Influenza virus A/CA07/09-(HA-NA)^{Min} increased signaling through B cell receptor suggesting induction of B cell response. Again, this observation was in accordance with our flow cytometry data showing elevated infiltration of B cells in tumors injected with the virus. Other upregulated pathways included processing and presentation of antigens in both MHCI and MHCII manner. This observation confirmed our hypothesis that treatment with Influenza virus A/CA07/09-(HA-NA)^{Min} activates immune system by enhanced exposure of tumor antigens by dendritic cells and priming cancer-specific response.

Among the pathways that were downregulated there were ones involved in receptor tyrosine kinases (RTKs) and mTOR signaling. Since signaling through RTKs such as EGFR is crucial for proliferation of many epithelial tumors while both TGFb and mTor pathways are involved in cancer metastasis such data suggested that treatment with Influenza virus A/CA07/09-(HA-NA)M not only inhibits primary tumor but also may have anti-metastatic potential.

Looking at the expression of the individual genes the strongest induced ones were the genes involved in T cell activation and signaling such as CD3, CD28 and CD247. CD86, a marker of activated T cell was also significantly upregulated. This suggests that treatment with Influenza virus A/CA07/09-(HA-NA)^{Min} stimulates activation of T cells which is crucial for development of adaptive anti-tumor response. Moreover, a set of genes involved in cytotoxic killing and associated with NK cells was also identified such as Granzymes A and B, Fas Ligand and Nkg7. Also, CCL5 (RANTES), a chemokine secreted by activated T lymphocytes and simulating proliferation of NK cells was strongly upregulated in the virus treated samples. Together such expression profile confirms our belief that activation of immune system by A/CA07/09-(HA-NA)^{Min} induces T and NK cell-mediated cytotoxicity that can be directed against tumor cells. It also supports our flow cytometry data showing that virus treated samples contain increased numbers of NK cells and T lymphocytes and elevated expression of CD86 on the surface of T cells.

When looking at downregulated genes EGFR and MMP9 were among the ones with the most decreased expression. EGFR is main receptor for EGF signaling and crucial factor in proliferation of multiple types of epithelial cancer. MMP9 is a secreted metalloproteinase responsible for release of growth factors and extracellular matrix (ECM) remodeling. It also promotes tumor cells invasion and metastasis. Downregulation of these genes is probably related to increased killing of cancer cells by immune system and shows that treatment Influenza virus A/CA07/09-(HA-NA)^{Min} effectively reduces tumor growth and metastatic potential.

Induction of anti-tumor immunity usually invokes negative loop of suppressor mechanisms. Indeed, in treated samples we saw significant upregulation of Pdcd1 and PdcdlLg2 coding for PD1 receptor and its ligand respectively. Additionally, Idol enzyme involved in tryptophane metabolism and immunosuppression was also upregulated. Although induction of these genes most likely decreases efficacy of our therapy, it can be overcame by combining Influenza virus A/CA07/09-(HA-NA)^{Min} with PD1/PDL1 inhibitors. Interestingly, while both PD1 and Idol are strongly induced by IFNg, we did not see any expression of IFNg in our samples. Indeed, analysis of raw data show that signal from IFNg specific probes was extremely low compared to the background (data not shown). Considering that two other genes that were highly upregulated in our assay (CXCL9 and CXCL10) are strongly dependent on IFNg and that we could detect IFNg produced by T cells reacting to EMT6 antigens in ELISPOT (exp MS87) and IFNg RNA via qPCR (MS72) lack of IFNg expression is perplexing and may be the result of technical issue with the probe.

In the next step we evaluated gene expression in tumors that received just a single dose of CodaLytic. The aim of this analysis performed on RNA collected 12 h after virus injection was to show what are the early effects of A/CA07/09-(HA-NA)^{Min} administration. While after 5 doses we can observe developed anti-tumor immunity, it is the time after first dose which is crucial for activation of immune system and building up adaptive response. Analysis of pathways showed that interferon signaling is the most affected by the virus and indeed top 3 most upregulated cellular genes (Ifit3, Oasl1, Rsad2) were interferon stimulated genes. Interestingly, even as early as 12 h after single virus administration we could see enhanced signaling through B and T cell receptor and increasing processing antigens through MHCI pathway. Such result suggest that induction of adaptive response can be initiated very early after virus administration which is crucial for mounting quick anti-cancer immunity. Also, we observed downregulation of mTOR pathway which may suggest that even before adaptive response is established Influenza virus A/CA07/09-(HA-NA)^{Min} can effectively impede ability of cancer cells to proliferate and metastasize.

Overall analysis identified over ˜150 cellular genes expression fold exceeding 1.5× and p value <0.05. In addition to interferon stimulated it included markers for monocytes/macrophages (CD163, CD200R1) elements of antigen presenting machinery (Tap2, H2-Q2), cytokines and chemokines (IL15, eotaxin-1, CCL9) and NFkb transcription factor. In contrast, H2-Aa, member of MHCII presentation machinery was strongly (over 6× downregulated). Such result could suggest that infection with Influenza virus A/CA07/09-(HA-NA)^{Min} pushes antigen presentation towards MHCI rather than MHCII pathway, an effect which is highly desired for induction anti-tumor immunity. Together these data present the picture of initial infection that induces interferon response, attracts myeloid cells and increases presentation of antigens that exposes tumor-specific markers to the T cells. T cells maturate and execute cytotoxic response through their CD8+ arm while the CD4+ arm modulates response and attracts NK cells that support cytotoxic killing. In the end persistent adaptive anti-tumor response is formed and gene expression profile changes to the one that has been seen after 5 rounds of treatment.

In our final step we evaluated expression of viral genes in the infected tumors. After single administration, all the influenza genes were detected at significantly higher level than threshold. However, different RNAs were expressed at different rate with levels of NEP over 70× higher than PB1. Differential expression of influenza genes is a phenomenon known in nature and amount of RNA expressed is usually proportional to the amount of protein building up the mature virion. Since viral particles contain mostly 8 RNA molecules at 1:1 stoichiometric ratio such big differences in RNA levels for each gene suggest that there is an active replication going on in the tumors and viral genes are amplified according to the biological demand. Both deoptimized genes HAmin and NAmin were expressed at lower levels than genes matrix or nuclear proteins, suggesting that RNA stability of codon-pair deoptimized RNAs may be decreased. However, without comparison with RNA from wild-type virus administered under the same condition it is hard to draw definite conclusion. Possibility of viral replication should not raise safety concerns since our data indicate that Influenza virus A/CA07/09-(HA-NA)^{Min} is completely cleared from tumors and does not spread to other organs.

A striking observation was that in tumors undergoing 5 rounds of treatment, levels of Influenza virus A/CA07/09-(HA-NA)^{Min} RNA were dramatically lower than in samples that received only a single dose. In fact, in group treated with 5 doses no viral RNAs except NS1 was present at levels statistically higher than in the MOCK group just 12 h after virus administration. Moreover, no genes related to interferon pathway that could be triggered in response to the virus were induced in those tumors. Such outcome suggested that repetitive administration of Influenza virus A/CA07/09-(HA-NA)^{Min} produces potent anti-viral immune response within just 10 days from initial treatment. Resulting immunity can neutralize the virus within short time even if it is applied at dose such high as 10⁸ PFU. This effect may raise concern whether anti-flu immunity can impede therapeutic effect of the virus. However, our studies of different therapeutic regimens in experiments MS64 and MS75 clearly showed that the best results with Influenza virus A/CA07/09-(HA-NA)^{Min} are obtained when it is administered several times for longer than the week. Therefore, the presence of anti-flu immunity does not seem to impair efficacy of therapy. It is possible that influenza-specific antibodies induce phagocytosis of opsonized viral particles by dendritic cells which in turn become activated by the viral RNA. Another positive aspect of effective anti-flu immunity that it should additionally address the safety concerns regarding spreading Influenza virus A/CA07/09-(HA-NA)^{Min} to other organs and inducing potential systemic infection.

Summarizing our data showed that treatment with Influenza virus A/CA07/09-(HA-NA)^{Min} induces potent anti-tumor immunity that is mainly executed by T lymphocytes and NK cells through their cytotoxic activity. Initial administration of the virus elicits strong interferon response leading to increased antigen presentation and priming components of adaptive immunity. With time effective anti-flu response is generated but it does not impair efficacy of therapy.

### Example 8

### Dose-Dependent Efficacy of Influenza Virus A/CA07/09-(HA-NA)^{Min} in a Murine EMT6 Breast Cancer Model

### Mice

Animal model: Mus musculus; Mouse Strain: Balb/c (Taconic); Age: 8-9 weeks old (Female); IACUC protocol: 2019-01-17-COD-1; Group size: 12

### Cells and Media

Cell line: EMT6 mouse triple-negative breast cancer (TNBC) cell line (ATCC, CRL-2755); Cell growth medium: Waymouth MB 752/1(Millipore Sigma, cat. no. W1625, lot no. SLCC1320) supplemented with 15% heat-inactivated fetal bovine serum (FBS, Gibco, cat. no. 10-082-147, lot no. 1982167); Cell concentration: 1×10⁵/50 uL (passage 4)

EMT6 cells were cultured at 37° C., 5% humidity and harvested using TrypLE Express (Gibco, cat. no. 12605-010) until cells detached from the culture flask. The trypsin reaction was stopped using Waymouth culture media with 15% FBS (see above). Cells were washed twice in phosphate buffered saline (PBS, Gibco, cat. no.14190-136), counted using a hemocytometer and Trypan Blue (Gibco, cat. no. 15250061) and cell viability was over 98%. EMT6 cells were centrifuged at 2,000 rpm for 5 min and resuspended to a concentration of 1×10^{5/50} uL in serum free Waymouth medium.

### Tumor Implantation and Treatment

Balb/C mice were anesthetized with 50 μL of a mixture of 50 mg/mL ketamine (Sigma, cat. no. K2753) and 5 mg/mL xylazine (Sigma, cat. no. X1251) injected intraperitoneally as approved by the Institutional Animal Care and Use Committee (IACUC), shaved around inguinal mammary fat pads and injected orthotopically with 50 μL of tumor cell suspension into the left fat pad. Animals were monitored daily for palpable tumors. On day 6 post implantation, tumors were palpable in all mice. Mice were assigned to 4 different treatment groups and injected intratumorally with 50 μL of L15 media (control) or influenza virus A/CA07/09-(HA-NA)^{Min}. Intratumoral injections were repeated on days 8, 10, 13, 15, 17, 20, 22, 24, 27, 29, and 31 post implantation, unless tumors completely resolved or the animal had to be euthanized sooner. This equates to treatment three times week (TIW) for up to 4 weeks.
Virus stock: Influenza virus A/CA07/09-(HA-NA)^{Min}; Lot 1-071621-1, 4×109 PFU/ml in L15 medium (Gibco, cat no.11415-064)
Virus stock dilution: 2× for highest dose group and 2 10× serial dilution for the middle and low dose groups, all in L15 medium (Gibco, cat no.11415-064)

Body weights and tumor volumes of both primary inguinal mammary fat pad tumors were measured three times a week until day 62 post implantation. Tumor growth was monitored by measurements of three perpendicular axes (A, B, C) with calipers and the volume was calculated using the following formula: V=0.52*ABC (ellipsoid volume).

Animals were anesthetized if tumors exceeded 500 mm³ of volume, in case of severe tumor ulceration or if weight loss exceeded 20%, in accordance with the IACUC protocol. Survival was recorded until day 62 post implantation.

### Data Analysis

Tumor volumes from each experimental group at each day of measurement were averaged using mean and standard deviation. Tumor growth inhibition (TGI) on day 20, i.e. when the first animals had to be sacrificed due to protocol-defined euthanasia criteria, are calculated using the following formula:

(1−(mean volume of treated tumors)/(mean volume of control tumors))×100[%]

Differences in tumor growth in fat pads over time were assessed using two-way ANOVA with Geisser-Greenhouse correction and Tukey's multiple comparisons test calculated in GraphPad Prism v9.1.0. Tumor volumes on selected days were compared using ordinary one-way ANOVA with Tukey's multiple comparisons test.

Animal survival was compared using log-rank (Mantel-Cox) test for pairwise comparisons and p values were adjusted using Bonferroni correction.

### Results

To confirm appropriate randomization of animals into 4 different regimen groups, tumor volumes on day 6 (FIG. 30). Mean tumor volumes ranged between 30.56 mm³ and 32.42 mm³ and were not statistically significantly different from another.

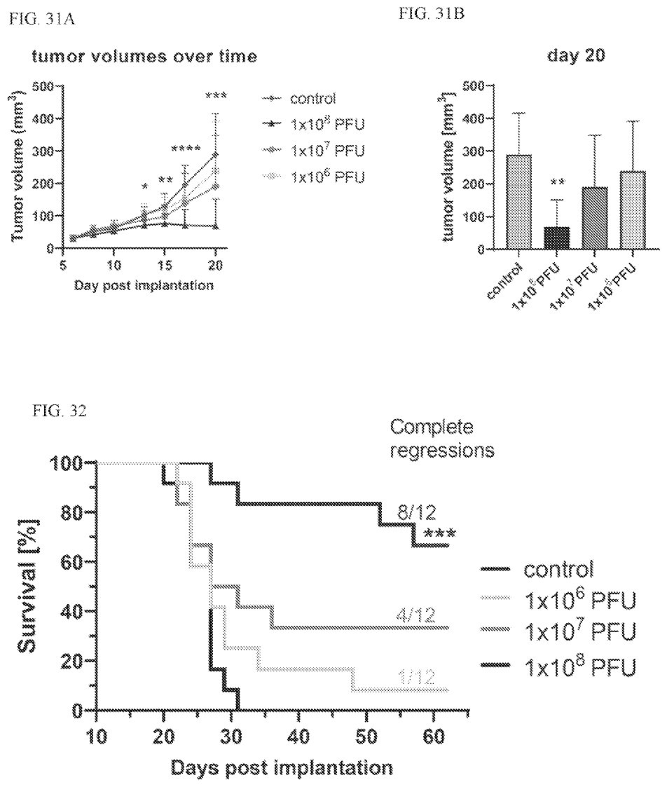

Treatment of EMT6 tumors in the inguinal mammary fat pad with intratumorally injections of influenza virus A/CA07/09-(HA-NA)^{Min} at doses higher than 1×10⁶ PFU led to tumor growth retardation in comparison to MOCK treated tumors (FIG. 31).

The strongest tumor inhibitory effect was observed in mice treated with 1×10⁸ PFU dose with 76.1% tumor growth inhibition (TGI) and 8/12 animals with complete tumor regression (p<0.001 vs control). Animals treated with 1×10⁷ PFU displayed weaker therapeutic effect (34.2% TGI, 4/12 complete regression, p=0.03 vs control), while mice treated with 1×10⁶ PFU showed no meaningful tumor growth inhibition compared to control-treated animals (17.1% TGI, 1/12 complete regression, p=0.16).

When comparing animals treated with Influenza virus A/CA07/09-(HA-NA)^{Min} or control treatment, the dose-dependent tumor growth inhibition translated into a dose-dependent survival benefit (FIG. 32): In absence of significant tumor growth inhibition in the 1×10⁶ PFU group, no survival benefit over the control group was observed. Increasing impact of tumor growth inhibition at the higher dose levels with increasing numbers of complete regressions resulted in a survival benefit with 4 and 8 long-term survivors beyond 50 days in the 1×10⁷ PFU and 1×10⁸ PFU groups, respectively.

At the end of the observation period on day 62, animal survival was as follows:

| | | |
|---|---|---|
| | Long-term | Complete |
| Group | survivors | regression |

| | | | | |
|---|---|---|---|---|
| Control | 0/12 | (0%) | 0/0 | (0%) |
| 1 × 10⁶ PFU LP | 1/12 | (8.3%) | 1/12 | (8.3%) |
| 1 × 10⁷ PFU LP | 4/12 | (33%) | 4/12 | (33%) |
| 1 × 10⁸ PFU LP | 8/12 | (66%) | 8/12 | (66%) |

### Summary and Conclusions

In this experiment, different doses of influenza virus A/CA07/09-(HA-NA)^{Min} were assessed in order to determine the required dose for intratumoral injection.

Comparison of different doses of low passage virus demonstrated that the 1×10⁸ PFU was the most effective dose when comparing the same material, leading to tumor regression in 66% of mice. The therapeutic effect of the 1×10⁷ PFU dose was lower with 33% mice clearing tumors and very limited effect was observed with the 1×10⁶ PFU dose with only 8% animals showing complete tumor regression. In this study, no prozone effect was observed that would result in reduced efficacy at doses beyond a particular optimal threshold.

In summary, influenza virus A/CA07/09-(HA-NA)^{Min}, the virus contained in the CodaLytic drug product, was able to significantly reduce tumor growth and increase survival after intratumoral injection with the optimal dose of 1×10⁸ PFU using a 4×TIW dosing regimen.

### Example 9

### Analysis of the Tumor Immune Infiltrate in Influenza Virus A/CA07/09-(HA-NA)^{Min} Treated EMT6 Tumors

### Mice

Animal model: Mus musculus; Mouse Strain: Balb/c (Taconic); Age: 8-9 weeks old (female); IACUC protocol: 2019-01-17-COD-1; Group size: 10 Cells and media
Cell line: EMT6 mouse triple-negative breast cancer (TNBC) cell line (ATCC, CRL-2755); Cell growth medium: Waymouth MB 752/1(Millipore Sigma, cat. no. W1625, lot no. SLCC1320) supplemented with 15% heat-inactivated fetal bovine serum (FBS, Gibco, cat. no. 10-082-147, lot no. 1982167); Cell concentration:1×10^{5/50} uL (passage 4)

EMT6 cells were cultured at 37° C., 5% humidity and harvested using TrypLE Express (Gibco, cat. no. 12605-010) until cells detached from the culture flask. The trypsin reaction was stopped using Waymouth culture media with 15% FBS (see above). Cells were washed twice in phosphate buffered saline (PBS, Gibco, cat. no.14190-136), counted using a hemocytometer and Trypan Blue (Gibco, cat. no. 15250061) and cell viability was over 98%. EMT6 cells were centrifuged at 2,000 rpm for 5 min and resuspended to a concentration of 1×10^{5/50} uL in serum-free Waymouth medium.

### Tumor Implantation and Treatment

Balb/c mice were anesthetized with 50 μL of a mixture of 50 mg/mL ketamine (Sigma, cat. no. K2753) and 5 mg/mL xylazine (Sigma, cat. no. X1251) injected intraperitoneally as approved by the Institutional Animal Care and Use Committee (IACUC), shaved around inguinal mammary fat pads and injected orthotopically with 50 μL of tumor cell suspension into the left fat pad. Animals were monitored daily for palpable tumors. On day 6 post implantation, tumors were palpable in all mice. Mice were assigned to 3 different treatment groups and injected intratumorally with 50 μL of L15 (control) or influenza virus A/CA07/09-(HA-NA)^{Min}. Intratumoral injections were repeated on days 8, 10, 13, and 15 post implantation.
Virus stock: Influenza virus A/CA07/09-(HA-NA)^{Min}, lot 1-071621-1, 4×10⁹; PFU/ml in L15 medium (Gibco, cat no.11415-064); Virus stock dilution: 2× for highest dose group and 10× serial dilution for the 10⁷; PFU group, all in L15 medium

Body weights and tumor volumes of primary inguinal mammary fat pad tumors were measured three times a week. Tumor growth was monitored by measurements of three perpendicular axes (A, B, C) with calipers and the volume was calculated using the following formula: V=0.52*ABC (ellipsoid volume). Mice were sacrificed on day 16 of study (day 10 of treatment).

Tumor volumes for each experimental group at each day of measurement were averaged using means and standard deviations. Differences in tumor growth over time was assessed using two-way ANOVA with Geisser-Greenhouse correction and Tukey's multiple comparisons test using GraphPad Prism v9.1.2.

### Tumor Processing

Tumors were stored on ice in 5 mL of RPMI (Gibco, cat. no. 11875-093) supplemented with 2% FBS (Gibco, cat. no. 10082147, lot 1982167). Next, tumors were mechanically dissociated using a gentleMACS Dissociator (Miltenyi Biotec) in 5 mL of volume.

Fragments were collected, centrifuged at 2,000 rpm for 5 min and resuspended in 800 μL of RPMI supplemented with 2% FBS. Next, 40 μL of 2000 U/mL DNAse (vendor, cat. no. D5025) and 50 μL of 10 mg/mL collagenase IV (vendor, cat. no. C5138) were added per each sample and tumors were shaken at 200 rpm for 1 h at 37° C. 12 mL of ACK buffer (150 mM NH₄C1, KHCO₃ 10 mM Na₂ EDTA 0.1 mM, pH 7.3) were added per sample and incubated for 10 min to lyse the red blood cells. Cells were collected by centrifugation at 2,000 rpm for 5 min and resuspended in 12 mL of RPMI supplemented with 10% FBS. Single cell suspensions were ensured by filtering subsequently through 70 μm and 30 um mesh filters (Miltenyi Biotec, cat. no.130-098-462 and 130-110-915).

### LIVE/DEAD Staining and Fc Blocking

After filtration, cells were collected by centrifugation at 2,000 rpm for 5 min and resuspended in 800 μL of PBS (Gibco, cat. no. 14190-136) containing reconstituted LIVE/DEAD AF700 nm dye (BD Biosciences, cat. no. 564997) at 3500× dilution. Cells were incubated for 15 min on ice in the dark. Next, cells were pelleted by centrifugation at 2,000 rpm for 5 min, resuspended in 250 μL of FACS buffer (0.5% bovine serum albumin (Lampire Biological Laboratories cat. #7500812) in PBS with 0.05% sodium azide (Sigma, cat. no. S2002) stored at 4° C. away from light) with 5 μL of anti-CD16/32 antibody (Biolegend, cat. no. 101320) added to block Fc receptors on the leukocytes. Samples were incubated on ice in the dark for 30 min.

Staining with Antibodies

After blocking, each sample was added to a tube containing antibody mastermix (see table below). Additionally, single staining and unstained controls were set up using counting beads (Beckman Coulter, cat. no. b22804). Antibodies were incubated for 30 min on ice in the dark. Next, cells were washed once with FACS buffer by adding 800 μL of buffer and centrifuging at 2,000 rpm for 5 min, the pellets were resuspended in 250 μL of FACS buffer and 250 μL of Fixing Solution (1% paraformaldehyde (PFA) in FACS buffer, generated by diluting a 10% neutral buffered formalin stock solution containing 4% total PFA (TissuePro, cat. #NBF03-32R) 1:4 in FACS buffer) was added per sample. Samples were stored at 4° C. in the dark until analysis by flow cytometry.

Samples were acquired using a LSRII Flow Cytometer and BD FACSDiva™ software. Antibodies that were used per sample (all BioLegend):

| | | Volume used per | |
|---|---|---|---|
| Antibody | Clone | sample (uL) | Target |
| anti-CD8a-BV605 | 53-6.7 | 2 | cytotoxic T cell marker |
| anti-CD4-PerCy5 | Gk1.5 | 0.8 | CD4+ T cell marker |
| anti-F4/80-APC | BM8 | 2 | macrophage marker |
| anti-CD49b-PE | DX5 | 1 | NK cells marker |
| anti-CD11c-BV421 | N418 | 2 | marker for activated dendritic |
| | | | cells and monocytes |
| anti-CD45-FITC | 30-F11 | 1 uL of 2x diluted | general leukocyte marker |
| anti-Ly6G-BV711 | 1A8 | 2.5 uL of 4x diluted | granulocyte marker |
| anti-Ly6C-PE-Cy7 | Hk1.4 | 1 uL of 4x diluted | monocyte marker |
| anti-CD19-APC-Cy7 | 6D5 | 2 uL of 2x diluted | B cell marker |
| anti-MHCII-BV650 | M5/114.15.2 | 2 ul of 10x diluted | marker for antigen-presenting |
| | | | cells, in particular macrophages |
| | | | and dendritic cells |
| anti-CD3e-BV785 | 145-2C11 | 2 | T cell marker |

### Gating and Analysis:

Flow cytometry data was analyzed using BD FACSDiva™ software. In brief, LIVE/DEAD staining was used to distinguish the population of living cells with high LIVE/DEAD signal intensity indicating dead cells/debris rejected from analysis. Within the alive population, CD45+ cells (all the leukocytes) and CD45− cells (predominantly cancer cells and other stromal cell types) were separated based on single staining and unstained controls.

Within the CD45+ leukocyte population, the following marker combinations were used to define individual cell types:

| | |
|---|---|
| CD3+ CD8+ | Cytotoxic CD8+ T cells |
| CD3+ CD4+ | CD4+ T cells |
| CD49b+ | Natural killer cells |
| CD19+ | B cells |
| Ly6C+Ly6G+ | Granulocytes |
| Ly6G− Ly6C+ | Monocytes |
| CD11c+ F4/80+ | Macrophages |
| CD11c+ MHCII+ | Dendritic cells |
| CD86+ CD11c+ MHCII+ | Activated dendritic cells |

Frequencies of cell populations across all samples were exported and further analyzed using GraphPad Prism v9.1.2. Cell frequencies per animal were used to calculate infiltration frequencies per treatment group described by mean and standard deviation. Differences in infiltration were determined using one-way ANOVA with Tukey's multiple comparisons test.

### Results

Intratumoral treatment with 10⁸ PFU of influenza virus A/CA07/09-(HA-NA)m resulted in a significant decrease in tumor size (p<0.05 on days 13 and 15 post implantation), confirming the onset of treatment effects at the time of tumor harvest on day 16.

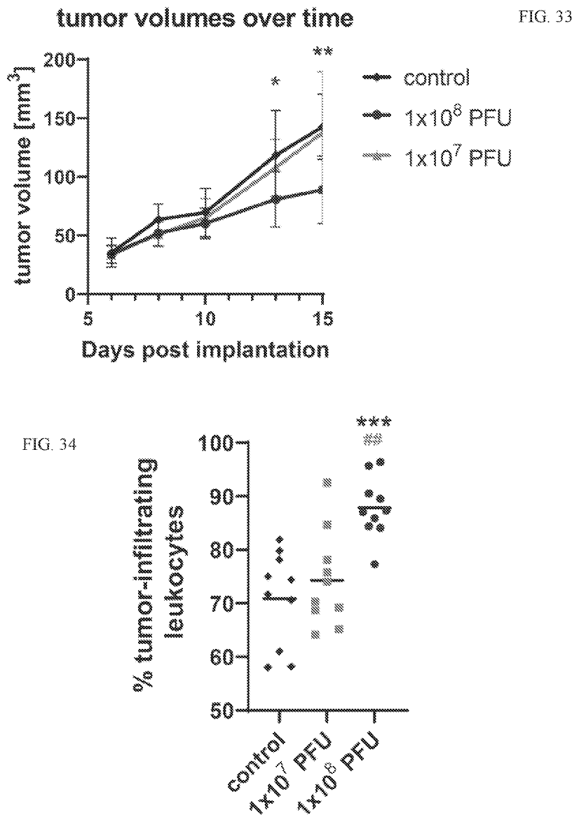

Treatment with 10⁷ PFU dose did not produce significant therapeutic effect over this time period (FIG. 33).

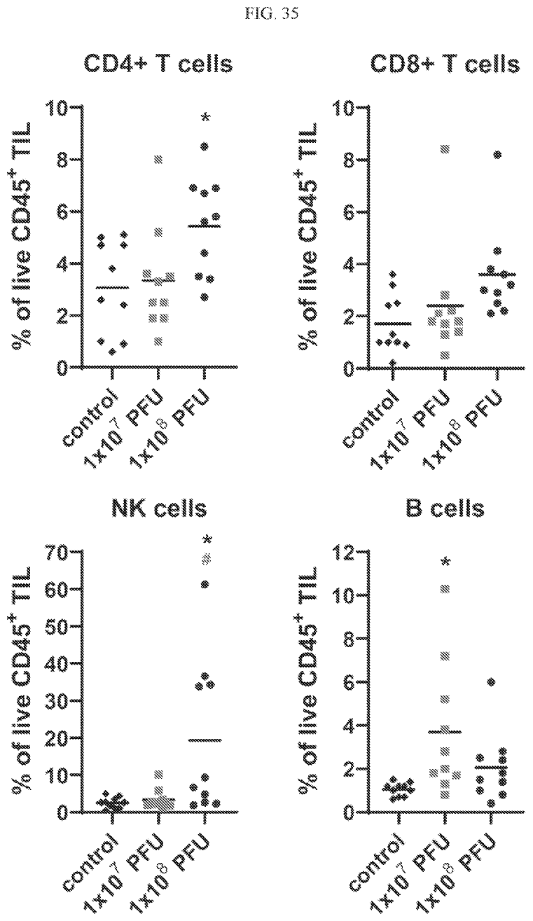

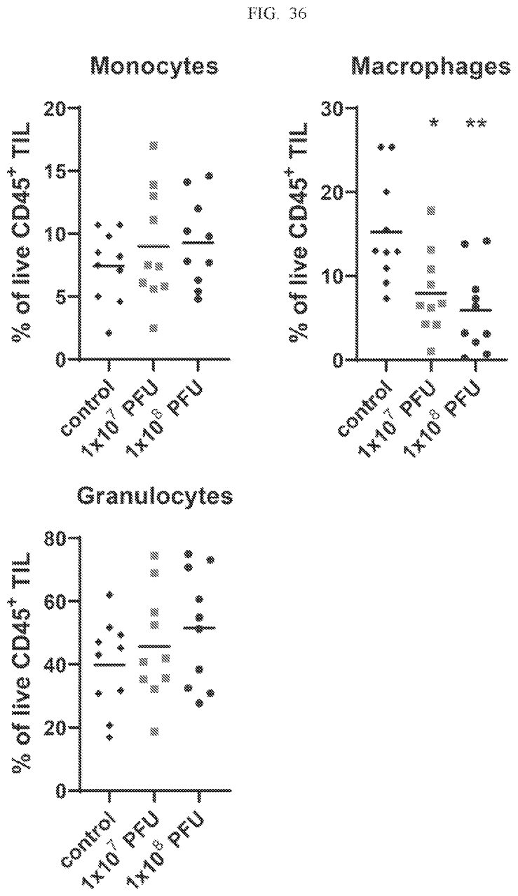

Immune cell infiltration after treatment is shown in FIGS. 34-37. In all animals, robust total immune cell infiltration was observed with 58.2-96.4% of all live cells staining positive for CD45 (FIG. 34). Tumors contained significantly larger tumor-infiltrating leukocytes (TIL) when treated with 1×10⁸ PFU influenza virus A/CA07/09-(HA-NA)^{Min} as compared to control treatment. At the 1×10⁷ PFU dose, no meaningful increaser in infiltration was observed.

Frequencies of immune cells of the lymphoid lineage increased in influenza virus A/CA07/09-(HA-NA)^{Min}-treated tumors compared to control-treated tumors (FIG. 35). At the higher dose of A/CA07/09-(HA-NA)^{Min}, statistically significant increases of CD4 T cell and NK cell infiltrates were observed (1.8-fold and 7.7-fold, respectively). CD8+ cytotoxic T cells infiltrates increased slightly and in a dose-dependent fashion after virus treatment in comparison to control, however did not reach statistical significance (2.1-fold and 1.4-fold over control for high and low dose treatment, respectively). B cell infiltrates increased after virus treatment (3.5-fold and 2.0-fold over control for high and low dose treatment, respectively), reaching significance at the 1×10⁷ PFU dose.

In the myeloid lineage, frequencies of monocytes and granulocytes did not significantly change after treatment with influenza virus A/CA07/09-(HA-NA)^{Min} (FIG. 36). In contrast, virus treatment significantly decreased frequencies of macrophages (2.6-fold, p<0.001 in the 1×10⁸ PFU group, 1.9-fold, p<0.05 in 1×10⁷ PFU group). The antibodies used in this experiment do not allow for further distinction between M1-like anti-tumor ad M2-like suppressive macrophages.

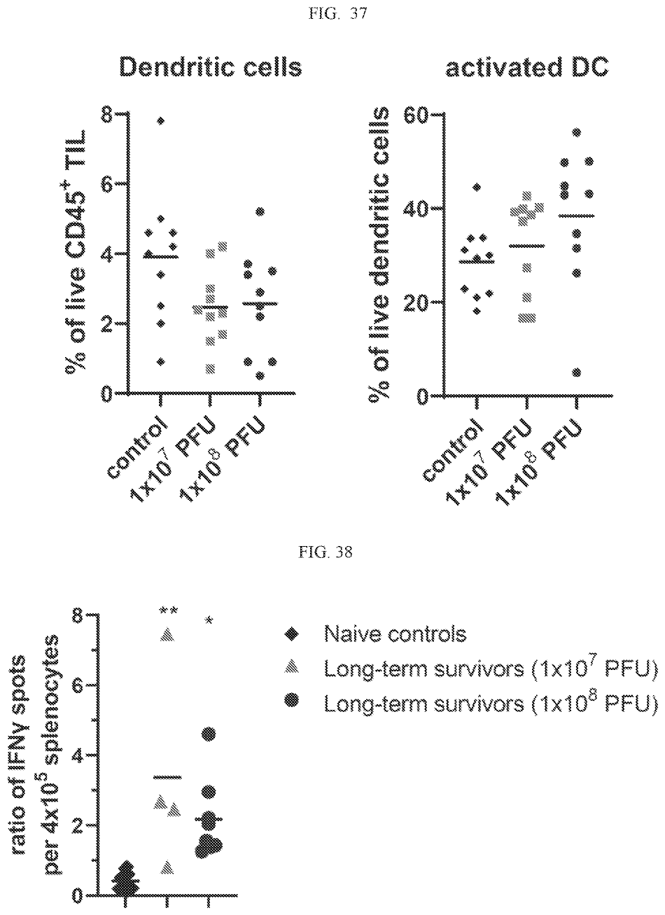

Tumors treated with treatment with A/CA07/09-(HA-NA)M^{Min} influenza virus contained slightly decreased frequencies of dendritic cells (1.5-fold in the 1×10⁸ PFU group and 1.6-fold in the 1×10⁷ PFU group; FIG. 37). When assessing the activation status of the tumor-infiltrating dendritic cells using CD86 surface expression as a marker of activation, a modest dose-dependent increase was observed after virus treatment (1.3-fold in the 1×10⁸ PFU group and 1.1-fold in the 1×10⁷ PFU group), suggesting a change in the quality of dendritic cells induced by treatment with influenza virus A/CA07/09-(HA-NA)^{Min}.

### Summary and Conclusions

In this experiment, pharmacodynamic changes in the tumor microenvironment induced by intratumoral treatment with influenza virus A/CA07/09-(HA-NA)^{Min} were characterized. Orthotopically implanted EMT6 triple-negative breast tumors were treated with 1×10⁸ or 1×10⁷ PFU of virus three times a week for a total of 5 doses, before immune cell infiltrates were quantified by flow cytometry. Tumors were collected for analysis on day 19 after onset off treatment, before regressing tumors were eliminated to enable the analysis.

Over the 10 days of treatment, tumor volumes decreased modestly in a dose-dependent fashion, confirming the onset of efficacy at the time point chosen for characterization of the immune cell infiltrate.

At the 1×10⁸ PFU dose, the total immune cell infiltrate significantly increased, suggestion the ability of influenza virus A/CA07/09-(HA-NA)^{Min} to convert colder tumors with low immune infiltration into warmer tumors with leukocytes infiltrating the tumor mass. Qualitatively, the immune cell infiltrate changed toward increased effector cell population, including T, B and NK cells. All of these cell populations have been implicated in anti-tumor immune responses either via direct anti-tumor effects in the case of NK cells or via antigen presentation and CD8⁺ T cell stimulating functions in the case of CD4+ T cells and B cells. Increased infiltration of activated dendritic cells, as observed in this experiment, further support the activity of these effector immune cells. In addition, influenza virus A/CA07/09-(HA-NA)^{Min} treatment decreased macrophage infiltration in tumors. Since tumor-associated macrophages are frequently M2 polarized and have immune-suppressive function, this virus-induced change may further enable the activity of the effector cells recruited to the tumor.

In summary, these data suggest that CodaLytic treatment induces innate and adaptive immune response mechanisms that lead to a more favorable, anti-tumor microenvironment, possibly contributing to anti-tumor efficacy. Analysis of the functional status of CD8 cytotoxic T cells and NK cells, the polarization of macrophage population and the differentiation status of the CD4+ T cells would further strengthen this observation.

### Example 10

### Protection from EMT6 Tumor Rechallenge in Long-Term Survivors After Influenza Virus A/CA07/09-(HA-NA)^{Min} Treatment

### Mice

Animal model: Mus musculus; Mouse Strain: Balb/C (Taconic); Age: 8-9 weeks old (Female); IACUC protocol: 2019-01-17-COD-1; Group size: 12 Cells and media
Cell line: EMT6 mouse triple-negative breast cancer (TNBC) cell line (ATCC, CRL-2755); Cell growth medium: Waymouth MB 752/1(Millipore Sigma, cat. no. W1625, lot no. SLCC1320) supplemented with 15% heat-inactivated fetal bovine serum (FBS, Gibco, cat. no. 10-082-147, lot no. 1982167); Cell concentration: 1×10⁵/50 uL for primary tumors (passage 4), 2×104/100 uL for secondary challenge (passage 8)

EMT6 cells were cultured at 37° C., 5% humidity and harvested using TrypLE Express (Gibco, cat. no. 12605-010) until cells detached from the culture flask. The trypsin reaction was stopped using Waymouth culture media with 15% FBS (see above). Cells were washed twice in phosphate buffered saline (PBS, Gibco, cat. no.14190-136), counted using a hemocytometer and Trypan Blue (Gibco, cat. no. 15250061) and cell viability was over 98%. EMT6 cells were centrifuged at 2,000 rpm for 5 min and resuspended to a concentration of 1×10⁵/50 uL in serum free Waymouth medium for primary tumor implantation. For rechallenge, cells were resuspended to a concentration of 2×10⁴/100 uL in serum free RPMI 1640 medium (Gibco, cat. no. 21875034).

### Primary Tumor Implantation and Treatment

Balb/C mice were anesthetized with 50 μL of a mixture of 50 mg/mL ketamine (Sigma, cat. no. K2753) and 5 mg/mL xylazine (Sigma, cat. no. X1251) injected intraperitoneally as approved by the Institutional Animal Care and Use Committee (IACUC), shaved around the inguinal mammary fat pads and injected orthotopically with 50 μL of tumor cell suspension into the left fat pad. Animals were monitored daily for palpable tumors. On day 6 post implantation, tumors were palpable in all mice. Mice were assigned to 2 treatment groups and injected intratumorally with 50 μL of PBS (control) or influenza virus A/CA07/09-(HA-NA)^{Min}, the virus in the CodaLytic drug product. Intratumoral injections were repeated on days 8, 10, 13, 15, 17, 20, 22, 24, 27, 29, 31 and 34 post implantation, unless tumors completely resolved or the animal had to be euthanized sooner.
Virus stock: Lot E2669/5 1-071619-3, 4×10¹⁰ PFU/ml; Virus stock dilution: 20× in PBS (Gibco, cat. no.14190-136); Control: PBS

Body weights and tumor volumes of primary inguinal mammary fat pad tumors were measured three times a week until day 64 post implantation. Tumor growth was monitored by measurements of three perpendicular axes (A, B, C) with calipers and the volume was calculated using the following formula: V=0.52*ABC (ellipsoid volume).

Animals were anesthetized if tumors exceeded 500 mm³ of volume, in case of severe tumor ulceration or if weight loss exceeded 20%, in accordance with the IACUC protocol. Survival was recorded until day 64 post implantation.

### Tumor Rechallenge

On day 87, the four long-term survivors as well as 12 naïve control animals were restrained using a tube, their tails were warmed up with a heat lamp and disinfected with 70% alcohol, and cells were injected via the lateral tail vein. Body weights were monitored three times a week. On day 16, when weight loss in 50% of control animals decreased below 90%, mice were sacrificed by CO₂ inhalation.

### Lung Assessment

Chests of euthanized animals were opened, and lungs were perfused by gradual injection of 10 mL of PBS into right ventricle of the heart. Lungs were removed, placed in buffered formalin (TissuePro, cat no. NBF03-32R) overnight and then transferred into 70% ethanol (Pharmco, cat. no. 111000200.) for storage at room temperature. Fixed lungs were visually examined, and tumor nodules were counted.

### Data Analysis

Tumor volumes from each experimental group at each day of measurement were averaged using mean and standard deviation. Tumor growth inhibition (TGI) on day 22, i.e. when the first animals in the control group had to be sacrificed due to protocol-defined euthanasia criteria, are calculated using the following formula:

(1−(mean volume of treated tumors)/(mean volume of control tumors))×100[%]

Differences in tumor growth in inguinal mammary fat pads over time were assessed using a mixed-effects model with Greisser-Greenhouse correction and Sidak's multiple comparisons test calculated in GraphPad Prism v9.1.0. Tumor volumes on selected days were compared using two-tailed unpaired t tests. Animal survival was compared using log-rank (Mantel-Cox) test.

Body weights after rechallenge were normalized to the weight on the day of cell injections and were compared over time using RM two-way ANOVA with Greisser-Greenhouse correction and Sidak's multiple comparisons test. The number of tumor nodules in lungs after rechallenge were compared using a two-tailed unpaired t test.

### Results

To confirm appropriate randomization of animals into groups, tumor volumes on day 6 were compared (FIG. 9). Mean tumor volumes ranged between 24.20 mm³ and 27.89 mm³ and were not statistically significantly different from another (p=0.53).

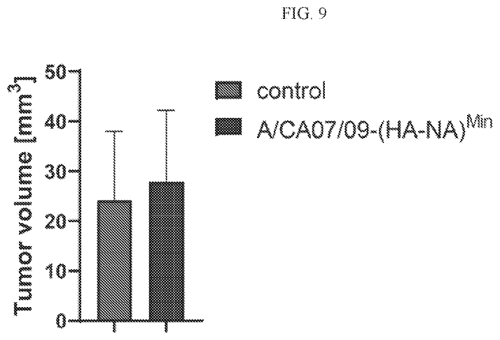

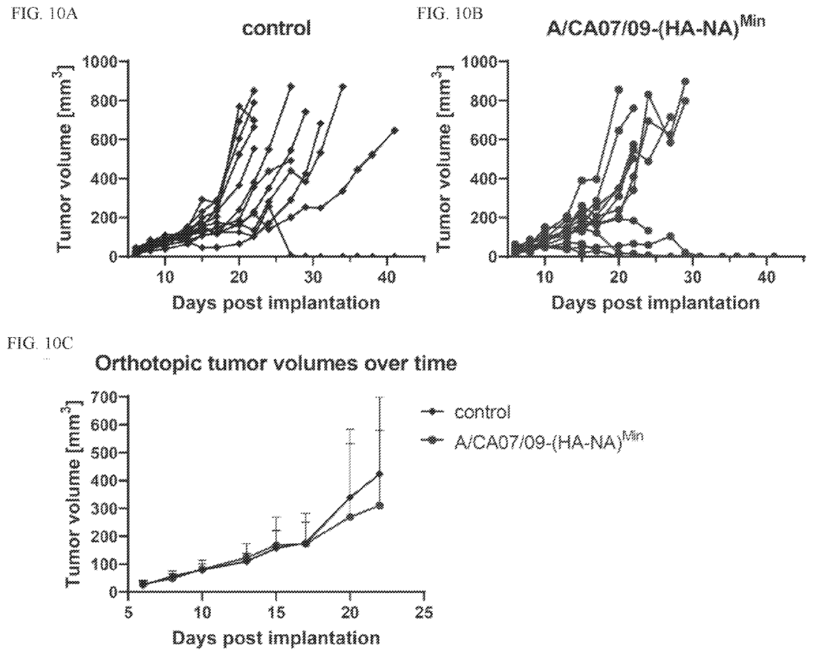

Treatment of EMT6 tumor with influenza virus A/CA07/09-(HA-NA)^{Min} led to moderate tumor growth inhibition (TGI at day 22: 26.9%) but did not lead to statistically significant tumor growth delay at this time point (FIG. 10).

Of note, one animal in the control-treated group rejected their tumor (animal C5) and one animal in the A/CA07/09-(HA-NA)^{Min}-treated group was euthanized due to ulceration despite regression of the tumor at that time (animal C4). However, over time the therapeutic effect of A/CA07/09-(HA-NA)^{Min} became more apparent, as reflected in the survival analysis. 4 animals treated with A/CA07/09-(HA-NA)^{Min} were alive and tumor-free by day 41 and remained in complete remission for the next two weeks (FIG. 11). This was in contrast to a single control-treated animals surviving (p=0.37).

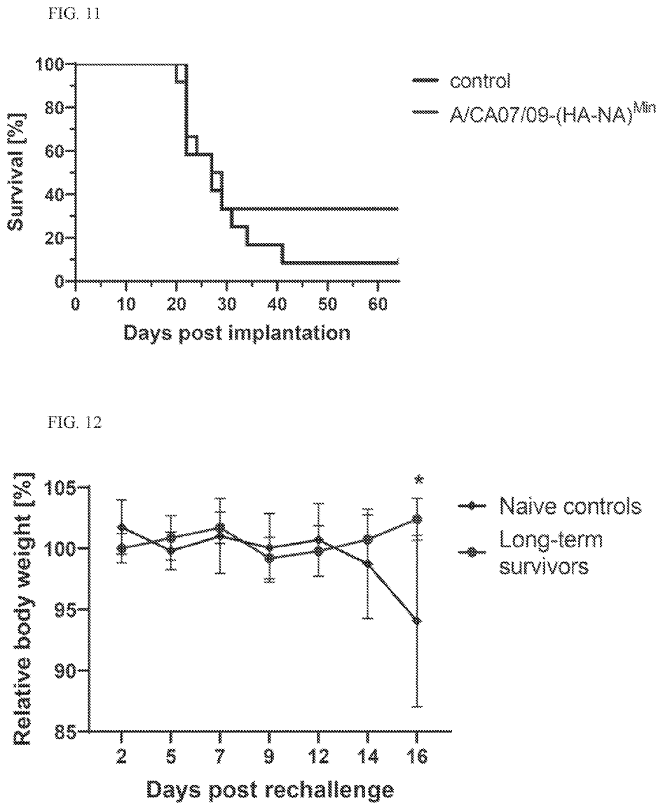

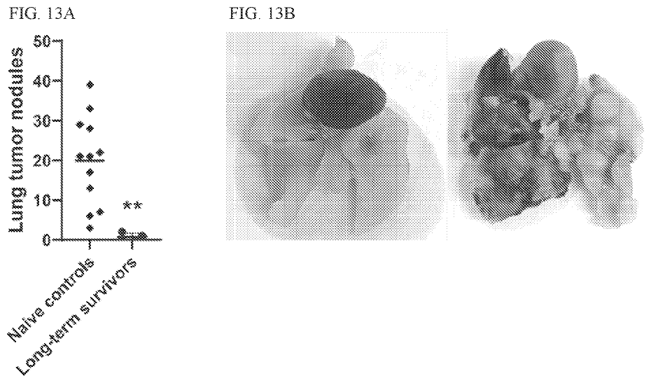

All four animals that were cured of their primary tumors by influenza virus A/CA07/09-(HA-NA)^{Min} treatment were rechallenged together with 12 tumor-naïve control mice. Body weight was monitored as a surrogate for lung colonization. By day 16 after rechallenge, 50% of control mice displayed more than 10% weight loss, indicating progressive growth of lung tumors (FIG. 12). At the same time point, none of the long-term survivors showed weight loss below their weight at time of rechallenge.

Lungs from naïve control animals contained a high number of nodules (mean 19.92, range 3 to 39), often completely covering the lungs and growing on a top of each other (FIG. 13). In contrast, the lungs of tumor survivors contained significantly fewer tumor nodules (mean 0.75, range 0 to 2, p=0.005). Half of the animals had no EMT6 tumors visible in their lungs.

### Summary and Conclusions

In this experiment, the efficiency of tumor formation after intravenous rechallenge in influenza virus A/CA07/09-(HA-NA)^{Min}-treated animals cured of their primary tumors was assessed. Primary tumors and rechallenge tumor cells were of the same EMT6 origin. Lung colonization was significantly reduced as compared to tumor challenge in naïve control mice with 50% of animals completely rejecting the injected tumor cells.

This data is supportive of the concept of anti-tumor immunity being generated as a bystander effect to A/CA07/09-(HA-NA)^{Min} treatment of orthotopic EMT6 tumors. The long-lasting immune response likely contributed to both primary tumor regression and rejection of challenge tumor cells in the lung.

### Example 11

### Efficacy of Influenza Virus a CA07/09-(HA-NA)^{Min} and Memory T Cell Reponses in a Dual EMT6Model

### Mice

Animal model: Mus musculus; Mouse Strain: Balb/C (Taconic); Age: 8-9 weeks old (Female); IACUC protocol: 2019-01-17-COD-1; Group size: 10 Cells and media
Cell line: EMT6 mouse triple-negative breast cancer (TNBC) cell line (ATCC, CRL-2755)
Cell growth medium: Waymouth MB 752/1(Millipore Sigma, cat. no. W1625, lot no. SLCC1320) supplemented with 15% heat-inactivated fetal bovine serum (FBS, Gibco, cat. no. 10-082-147, lot no. 1982167)
Cell concentration: 1×10^{5/50} uL (passage 4)

EMT6 cells were cultured at 37° C., 5% humidity and harvested using TrypLE Express (Gibco, cat. no. 12605-010) until cells detached from the culture flask. The trypsin reaction was stopped using Waymouth culture media with 15% FBS (see above). Cells were washed twice in phosphate buffered saline (PBS, Gibco, cat. no.14190-136), counted using a hemocytometer and Trypan Blue (Gibco, cat. no. 15250061) and cell viability was over 98%. EMT6 cells were centrifuged at 2,000 rpm for 5 min and resuspended to a concentration of 1×105/50 uL in serum free Waymouth medium.

### Tumor Implantation and Treatment

Balb/C mice were anesthetized with 50 μL of a mixture of 50 mg/mL ketamine (Sigma, cat. no. K2753) and 5 mg/mL xylazine (Sigma, cat. no. X1251) injected intraperitoneally as approved by the Institutional Animal Care and Use Committee (IACUC), shaved around mammary fat pads and injected orthotopically with 50 μL of tumor cell suspension into the left fat pad. Animals were monitored daily for palpable tumors. On day 6 post implantation, tumors were palpable in all mice. Mice were assigned to different treatment groups and injected intratumorally with 50 μL of either PBS (n=20 in total, groups “control E” and “control L”) or influenza A/CA07/09-(HA-NA)M (n=20 in total, groups “A/CA07/09-(HA-NA)^{Min}E” and “A/CA07/09-(HA-NA)^{Min} L”). Intratumoral injections were repeated on days 8, 10, 13, 15, 17, 20, 22, 24, 27, and 29 post implantation, until tumors were completely resolved or until the animal had to be euthanized.
Virus stock: Lot 3-080620-2, 2×¹⁰⁰ PFU/ml; Virus stock dilution: 10× in PBS (Gibco, cat. no.14190-136); Control: PBS

On days 9 (early, E; 10 control- and virus-treated animals each) and 13 (late, L; 10 control- and virus-treated animals each), secondary tumors were implanted subcutaneously into the right flank of all animals. Cells were cultured and prepared analogously to the initial tumor cell inoculate and implanted at a concentration of 1×10⁵/50 uL in serum free Waymouth medium. Manual restraint was used in place of anesthesia for these subcutaneous injections, as approved by the IACUC protocol.

Body weights and tumor volumes of both primary mammary fat pad and secondary flank tumors were measured three times a week. Tumor growth was monitored by measurements of three perpendicular axes (A, B, C) with calipers and the volume was calculated using the following formula: V=0.52*ABC (ellipsoid volume). Animals were anesthetized if either fat pad or flank tumor exceeded 500 mm³ of volume, in case of severe tumor ulceration or if weight loss exceeded 20%, in accordance with the IACUC protocol. Tumor growth on the flank was observed until day 24. Survival was recorded until day 50 post implantation.

### Mouse Splenocyte Isolation

Three mice in either of the two virus-treated groups that survived until day 50 (long-term survivors; animals VE8, VE10 and VL9) and three naïve Balb/C mice were sacrificed, their spleens resected and manually dissociated by grinding between two frosted microscope slides. Ground spleens were collected in 10 mL of RPMI (Gibco, cat. no. 11875-093), supplemented with 2% FBS and centrifuged at 2,000 rpm for 5 min. Spleen preparations were depleted of red blood cells by a 10 min incubation in 12 mL of ACK Lysing Buffer (Gibco, cat. no. A1049201) at room temperature. Next, ACK buffer was neutralized by adding 2× volume of 2% FBS RPMI. Cells were collected by centrifugation at 2,000 rpm for 5 min, resuspended in 10 mL of 2% FBS RPMI and filtered through 70 μm and 30 um mesh strainers (Miltenyi Biotec, cat. no.130-098-462 and 130-110-915). Splenocytes were counted with hemocytometer and Trypan Blue (Gibco, cat. no. 15250061) and cell viability was over 98%. Cells were pelleted by centrifugation at 2.00 rpm for 5 min and resuspended at a density of 1.5×10⁶/mL in CTL Test Medium (ImmunoSpot, cat. no. CTLT-005) supplemented with 1× penicillin/streptomycin (Gibco, cat. no. 15-140-122, final concentration 100 U/mL) and 1× GlutaMAX (Gibco, cat. no. 35050061).

### EWT6 Lysate for Ex Vivo Restimulation

One confluent 10 cm plate with 5×10⁶ of EMT6 cells, cultured at 37° C., 5% humidity, were harvested as described above and resuspended in 1 mL of CTL medium. Cells were lysed by 5 repeating freeze-thaw cycles at −80° C. and room temperature.

### Enzyme-Linked Immunosorbent Spot Assay (ELISpot)

To quantify IFNg-producing T cells in response to tumor cell lysate, a Murine IFNg Single-Color Enzymatic ELISpot Assay (ImmunoSpot, cat. no. mIFNg-1M/2) was used for this assay. 3×105 splenocytes were seeded at a density of 3×10⁵ per well in 200 uL in the assay plate included in the kit, prepared with capture solution and washed according to manufacturer's instruction. Cells were stimulated wither with 20 μL of EMT6 cell lysate in triplicates or 20 μL of CTL medium in duplicates (negative control). Cells were incubated for 24 h at 37° C. The plate was developed according to the manufacturer's protocol and the resulting spots were counted manually using a loupe. Each spot is equivalent to an IFNg-secreting T cell.

### Data Analysis

Tumor volumes from each experimental group at each day of measurement were averaged using mean and standard deviation. Tumor growth inhibition (TGI) on day 27 are calculated using the following formula:

(1−(mean volume of treated tumors)/(mean volume of control tumors))×100[%]

Differences in tumor growth in fat pads over time were assessed using a mixed-effects model with Geisser-Greenhouse correction and Tukey's multiple comparisons test calculated in GraphPad Prism v9.1.0. Tumor volumes on selected days were compared using ordinary one-way ANOVA with Tukey's multiple comparisons test.

Animal survival was compared using log-rank (Mantel-Cox) test for pairwise comparisons and p values were adjusted using Bonferroni correction.

Replicate ELISpot measurements were averaged using means and for each sample the signal in control-stimulated well was subtracted from the EMT6-stimulated conditions.

Negative background-subtracted values were set to 0. Data was plotted and analyzed using means, standard deviations and unpaired two-tailed t tests in GraphPad Prism v9.1.0.

### Results

To confirm appropriate randomization of animals into 4 different regimen groups, tumor volumes on day 6 (FIG. 19). Mean tumor volumes ranged between mm³ and 34.28 mm³ and were not statistically significantly different from another.

Treatment of primary tumors in the mammary fat pad with intratumorally injections of influenza virus A/CA07/09-(HA-NA)^{Min} led to tumor growth retardation in comparison to control treated tumors and independently of whether secondary tumors were implanted at the early or later timepoint (FIG. 20). On day 17, i.e. the first day an animal in the study had to be sacrificed as per study criteria and 11 days after onset of treatment, A/CA07/09-(HA-NA)^{Min}-treated tumors in animals who had flank tumor implanted on the late schedules were significantly smaller than control-treated tumors in either group (p=0.0324 for control E vs A/CA07/09-(HA-NA)^{Min} L and p=0.0088 for control L vs A/CA07/09-(HA-NA)^{Min} L). On day 27, when 40% of animals in both control-treated groups were evaluable for tumor size before half of those needed to be sacrificed, all comparison between control and A/CA07/09-(HA-NA)^{Min}-treated group, regardless of implantation date for secondary tumors, showed statistically significant tumor growth reduction after virus treatment (FIG. 20B). When tumor growth was compared over time, at most time points after onset of treatment, at least one combination of A/CA07/09-(HA-NA)M and control-treated groups achieved (E vs L) statistical significance (FIG. 20C). There was a trend toward increased tumor growth retardation after viral treatment with the late vs the early implantation regimen (tumor growth inhibition 96.2% vs 73.6% vs their respective control group on day 27), but these differences were not statistically significant at any time point.

Most secondary tumors implanted subcutaneously into flanks early (E, day 9) or late (L, day 13) did not grow. However, all tumors that did grow out were in animals that had received control treatment (4 tumors in the control E group and 3 tumors in the control L group vs no outgrowth in the A/CA07/09-(HA-NA)^{Min} E group and one regressing tumor in the A/CA07/09-(HA-NA)^{Min} L group, FIG. 21). There were no control animals included in this study to establish the usual take rate for flank EMT6 tumors with this specific tumor cell preparation, although outgrowth would be expected in more than 90% of tumor-naïve animals and viability of the implanted cells was >98%. Outgrowth of secondary tumors in animals with primary orthotopic tumors may have been impacted by generation of an anti-tumor immune response based on the presence of foreign tumor antigens, whose release may be facilitated by repeated intratumoral injections. This effect appeared to be increased in virus-treated animals, in which virus-mediated cell killing increased spread of tumor antigens and/or supported immune cell activation.

When comparing animals treated with A/CA07/09-(HA-NA)^{Min} or control treatment, tumor growth inhibition translated into a survival benefit (FIG. 22). Both A/CA07/09-(HA-NA)^{Min}-treated groups prolonged survival significantly (p<0.0001) as compared to their respective control controls. As for the tumor volumes, there was a trend toward longer survival in virus-treated animals that received their secondary tumors at the late timepoint in comparison those with secondary tumor implantation at the early timepoint (8 survivors vs 4 survivors on day 50, respectively), but again the difference did not reach statistical significance. All long-term survivors were tumor with the exception of one animal in the A/CA07/09-(HA-NA)^{Min} L group (7/8=88% of animals tumor-free). While euthanasia criteria included growth and ulceration phenotype of both mammary fat pad and flank tumors, all animals met that endpoint as the result of mammary fat pad tumor volume.

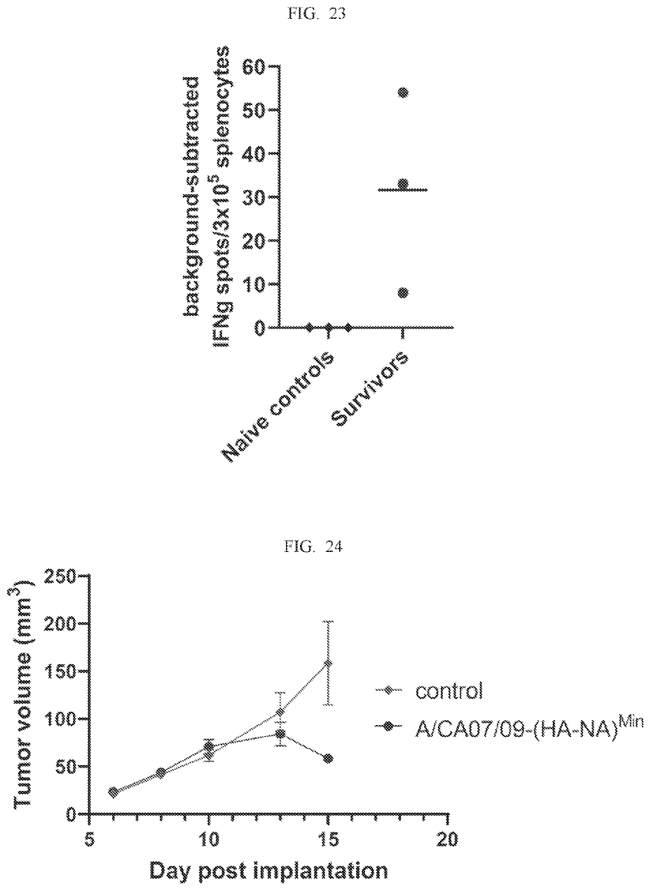

Three surviving mice were sacrificed on day 50 together with 3 naïve Balb/c mice and IFNg response of splenocytes after ex vivo restimulation with EMT6 tumor cell lysate was quantified by ELISpot (FIG. 23). Splenocytes from naïve animals, that had not been implanted with EMT6 tumor at any location did not show any IFNg response within 24 h, indicating that presence of foreign antigen was not sufficient to induce a de novo T cell response. In contrast, splenocytes from all three long-term survivors, previously treated with influenza virus A/CA07/09-(HA-NA)M^{u}, contained a cell population that responded to exposure to EMT6 lysates by IFNg secretion (mean 31.67 spots/3×10⁵ splenocytes, range 8-45 spots), suggesting a memory recall response against tumor antigens induced by intratumoral A/CA07/09-(HA-NA)^{Min} treatment.

### Summary and Conclusions

In this experiment, induction of anti-tumor immune responses by treatment with influenza virus A/CA07/09-(HA-NA)^{Min}, the virus contained in the CodaLytic drug product, was assessed by investigating primary and secondary tumor growth as well as T cell recall responses.

Viral treatment by intratumoral injection significantly slowed down growth of injected primary tumors in mammary fat pad and induced several complete and lasting tumor regressions. In addition, the growth of secondary tumors implanted in a delayed fashion into the flank of the mice was prevented while a proportion of secondary tumors in control-treated animals did grow out, suggesting an increase in anti-tumor immunity via epitope spread after treatment with A/CA07/09-(HA-NA)^{Min}.

This mechanism of action was further supported by presence of ex vivo recall responses against virus-free tumor cell lysates as measured by ELISpot, which were absent in splenocytes from naïve animals. Control-treated animals could not be used as a control for this restimulation experiment due to their rapid tumor growth requiring euthanasia, as would be the case in traditional rechallenge models, in which tumor growth in previously treated long-term survivors would be compared to tumor challenge in naïve, age-matched mice. The trend toward increased tumor growth inhibition and survival in the A/CA07/09-(HA-NA)^{Min}-treated animals with late as opposed to early secondary tumor implantation does not contradict the evidence collected here of induction of immune responses against tumor antigens by intratumoral A/CA07/09-(HA-NA)^{Min} treatment. In aggregate, this data supports that influenza virus A/CA07/09-(HA-NA)^{Min} induced analogous immune-stimulatory mechanisms of action.

### Example 12

### Evaluation of Memory T Cell Responses Induced by Treatment with Influenza Virus A/CA07/09-(HA-NA)^{Min} in EMT6Model

### Mice

Animal model: Mus musculus; Mouse Strain: Balb/C (Taconic); Age: 16-17 weeks old (Female); IACUC protocol: 2019-01-17-COD-1; Group size: 8 survivors that were treated with 10^{Min} PFU influenza virus A/CA07/09-(HA-NA)^{Min} survivors that were treated with 10⁷ PFU virus A/CA07/09-(HA-NA)^{Min} 12 naïve mice used as a control

### Cells and Media:

Cell lines: EMT6 mouse triple-negative breast cancer (TNBC) cell line (ATCC, CRL-2755); MDCK.2 canine epithelial kidney cell line (ATCC, CRL-2936); Cell growth media: Waymouth MB 752/1 (Millipore Sigma, cat. no. W1625, lot no. SLCC1320) supplemented with 15% heat inactivated fetal bovine serum (FBS, Gibco, cat. no. 10-082-147, lot no. 1982167); OptiProSFM (Gibco, cat no 12309019), supplemented with 1× GlutaMAX (Gibco, cat. no. 35050061)

### Mouse Splenocyte Isolation

Twelve mice that were cleared of EMT6 tumors following treatment with influenza A/CA07/09-(HA-NA)^{Min} in a prior efficacy experiment (eight treated with 10′ PFU and four mice treated with 10⁷ PFU, day 62 post tumor implantation) and twelve naïve Balb/C mice were sacrificed, their spleens resected and manually dissociated by grinding between two frosted microscope slides. Ground spleens were collected in 10 mL of RPMI (Gibco, cat. no. 11875-093), supplemented with 2% FBS and centrifuged at 2,000 rpm for 5 min. Spleen preparations were depleted of red blood cells by a 10 min incubation in 12 mL of ACK Lysing Buffer (Gibco, cat. no. A1049201) at room temperature. Next, ACK buffer was neutralized by adding 2× volume of 2% FBS RPMI. Cells were collected by centrifugation at 2,000 rpm for 5 min, resuspended in 10 mL of 2% FBS RPMI and filtered through 70 μm and 30 um mesh strainers (Miltenyi Biotec, cat. no.130-098-462 and 130-110-915). Splenocytes were counted with hemocytometer and Trypan Blue (Gibco, cat. no. 15250061) and cell viability was over 98%. Cells were pelleted by centrifugation at 2,000 rpm for 5 min and resuspended at a density of 2×10⁶/mL in CTL Test Medium (ImmunoSpot, cat. no. CTLT-005) supplemented with 1× penicillin/streptomycin (Gibco, cat. no. 15-140-122, final concentration 100 U/mL) and 1× GlutaMAX (Gibco, cat. no. 35050061).

### EMT6 Lysates for Ex Vivo Restimulation

One confluent 10 cm plate with 5×10⁶ of EMT6 cells, cultured at 37° C. and 5% humidity in Waymouth MB 752/1 growth media, were harvested using TrypLE Express (Gibco, cat. no. 12605-010) until cells detached from the culture flask. The trypsin reaction was stopped using Waymouth culture media with 15% FBS and cells were washed once with serum free Waymouth medium. Cells were resuspended in 1 mL of CTL Test Medium. Cells were lysed by 5 repeating freeze-thaw cycles at −80° C. and room temperature.

### Influenza-Infected MDCK2 Lysates for Ex Vivo Restimulation

Four 10 cm plates of MDCK.2 cells were cultured at 37° C. and 5% humidity in OptiPro SFM to reach 95% confluency before infection either with 4×10⁷ PFU of influenza A/CA07/09-(HA-NA)^{Min} (2 plates) or media alone (2 plates). 24 h post infection, cells were collected using TrypLE Express as described above for EMT6 cells. Cells were washed once with PBS (Gibco cat no. 14190-136) and resuspended in 2 mL of CTL medium/plate. Cells were lysed by 5 repeating freeze-thaw cycles at −80° C. and room temperature.
Virus stock: Lot 3-06-16-21-1, 1×10¹⁰ PFU/ml; Infection media: OptiPRO SFM (Gibco, cat. no. 12309019) supplemented with 0.2% bovine serum albumin (Lampire Biological Laboratories, cat. no.7500812)

### Enzyme-Linked Immunosorbent Spot Assay (ELISpot)

To quantify IFNg-producing T cells in response to tumor cell lysate, a Mouse IFN-γ ELISpot PLUS kit (ALP) (Mabtech, cat no. 3321-4APT-10) was used. Pre-coated plates were prepared according to manufacturer's instruction, before 4×10⁵ splenocytes were seeded in 200 uL per well. For studying anti-tumor immune responses, cells were stimulated either with 20 μL of EMT6 cell lysate or 20 μL of CTL medium (negative control) in triplicates. For studying anti-flu immunity, splenocytes were stimulated either with 20 ul of either influenza virus-infected MDCK.2 lysate or uninfected MDCK.2 lysate (negative control) in duplicates. Additional wells were stimulated in duplicates with 50 ng/mL phorbol myristate acetate (PMA, Invivogen, cat no. tlrl-pma) and 1 μg/mL ionomycin (Invivogen, cat no. inh-ion). Four naïve control samples and four samples from long-term survivors were arranged for each of the assay plates to avoid any potential plate-to-plate variability. Cells were incubated for 24 h at 37° C. and plates were developed according to the manufacturer's protocol

### Image Analysis and Statistics

Wells on the plates were photographed under the loupe using and the pictures were processed using ImageJ software v1.53j (National Institutes of Health and the Laboratory for Optical and Computational Instrumentation). First, images were converted to 8-bit grayscale and then the threshold was set to the value 175 to separate the spots from the background. Next, the Measure Particles function was used to calculate the number of spots with a size >150 pixels and circularity >0.1. Resulting ELISpot measurements across biological replicates were averaged using arithmetic means for each animal and stimulation condition.

For analysis of anti-tumor immune responses in each animal, mean spot counts from the EMT6-stimulated condition were divided by the number of spots in media control-stimulated wells. For analysis of anti-flu immunity, mean spot counts from the wells treated with influenza-infected MDCK.2 lysate were divided by the number of spots in the wells stimulated with non-infected MDCK.2 lysate. To detect immune responses against residual MDCK.2 components in the virus preparation, mean spot counts in response to uninfected MDCK.2 lysates were divided by mean spot counts in media control-stimulated wells. This comparison also contains a xeno-reaction component of mouse splenocytes to dog cell lysate.

The ratios above were compared between naïve mice and the two groups of EMT6-survivors using one-way ANOVA with Tukey's test for multiple comparison using GraphPad Prism v9.1.0.

### Results

After ex vivo stimulation of splenocytes with positive control stimuli PMA/iononmycin, that act independently of T cell receptor engagement via activation of protein kinase C and as calcium ionophore, respectively, IFNγ spot counts were too numerous to count for all samples, indicative of healthy cell preparations with functional T cells. As a result, data from all animals was included in further analysis.

To determine whether animals in which intratumoral treatment with influenza virus A/CA07/09-(HA-NA)^{Min} led to complete regression of orthotopically implanted EMT6 tumors, splenocytes collected on day 62 post tumor implantation were restimulated ex vivo with EMT6 cell lysates or media control. The ratio of EMT6 spots counts over background serves as an indicator of polyclonal anti-tumor immune responses (FIG. 38).

The number of IFNγ-producing cells was significantly higher in long-term survivors than in naïve control animals (8.1-fold in animals treated with 1×10⁷ PFU of influenza virus A/CA07/09-(HA-NA)^{Min}, p=0.002 and 5.2-fold in animals treated with 1×10⁸ PFU, p=0.017 as compared to naïve control animals). The difference between IFNγ spot formation in survivors that had been treated with the different doses of influenza virus A/CA07/09-(HA-NA)^{Min} was not statistically significant (p=0.3).

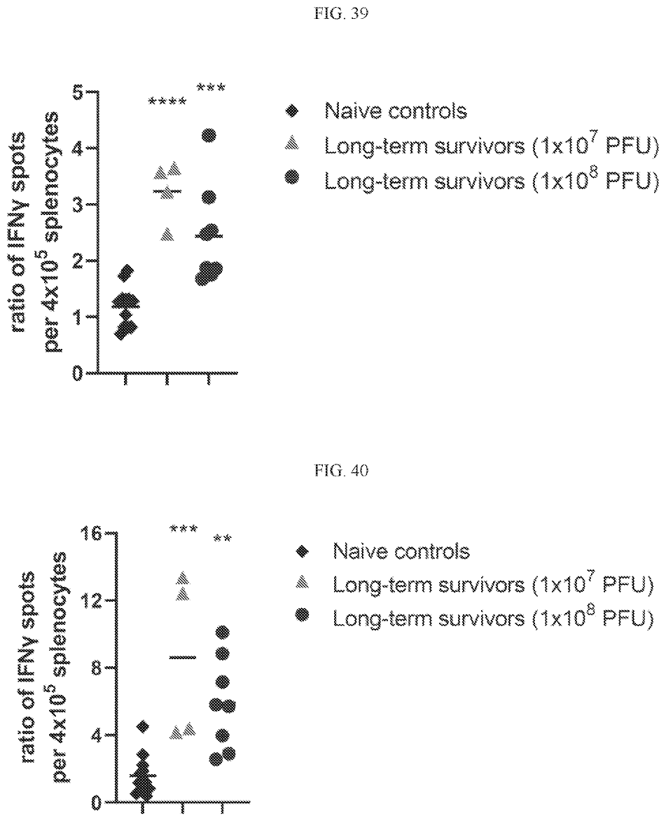

In addition to the anti-tumor immune responses, it is expected that animals treated with influenza virus A/CA07/09-(HA-NA)^{Min} also mount a cellular immune response against viral antigens. To quantify the magnitude of this response, splenocytes were restimulated with infected and uninfected MDCK.2 cell lysates. This cell line had been used to produce the research-grade material used for treatment of the EMT6 tumors in a prior efficacy experiment. Ratios of responses to infected over uninfected MDCK.2 cells were calculated to isolate the anti-viral recall response from any potential reaction to the MDCK.2 cells of dog origin (FIG. 39).

Anti-viral IFNγ recall responses were significantly higher in long-term survivors than in naïve control animals (2.7-fold in animals treated with 1×10⁷ PFU of influenza virus A/CA07/09-(HA-NA)^{Min}, p<0.0001 and 2.1-fold in animals treated with 1×10⁸ PFU, p=0.0005 as compared to naïve control animals). The difference between IFNγ spot formation in survivors that had been treated with the different doses of influenza virus A/CA07/09-(HA-NA)^{Min} was not statistically significant (p=0.1).

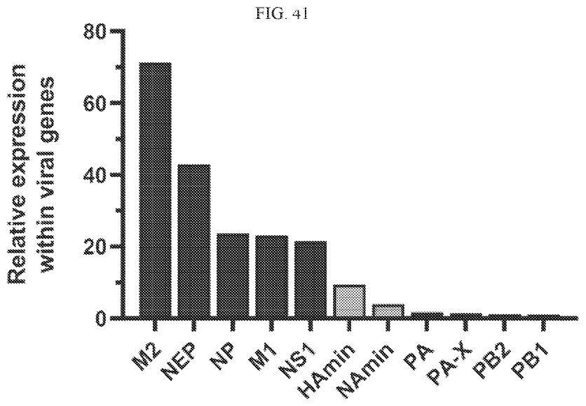

The data generated in the is experiment also allowed to determine whether cellular immune responses were mounted against any remained components of the MDCK.2 production cell line contained in the virus preparation used for treatment of EMT6 tumors. To this end, ratios of IFNγ response after restimulation with uninfected MDCK.2 cells over media control stimulation were compared (FIG. 40).

IFNγ responses to MDCK.2 cell lysate were significantly higher in long-term survivors than in naïve control animals (5.4-fold in animals treated with 1×10⁷ PFU of influenza virus A/CA07/09-(HA-NA)^{Min}, p<0.0004 and 3.7-fold in animals treated with 1×10⁸ PFU, p=0.004 as compared to naïve control animals). The difference between IFNγ spot formation in survivors that had been treated with the different doses of influenza virus A/CA07/09-(HA-NA)^{Min} was not statistically significant (p=0.2). Of note, the absolute ratios of IFNγ response of MDCK.2 cells over media control stimulation (means of 1.58 for naïve control animals, 8.60 for animals treated with 1×10⁷ PFU, and 5.88 for animals treated with 1×10⁸ PFU) include a combination of a xeno-response to dog MDCK.2 cells and recall responses to any MDCK.2 components in the virus-treated animals.

### Summary and Conclusions

In this experiment, induction of immune responses by treatment with influenza virus A/CA07/09-(HA-NA)^{Min}, the virus contained in the CodaLytic drug product, was assessed by quantification of T cell recall responses. Splenocytes were used here as a source for antigen-specific T cells and serve as a surrogate for peripheral T cells, as tumor-infiltrating lymphocytes cannot be collected after tumor clearance. While not formally proven by phenotypic characterization, the T cells responding to restimulation in this experiment could be considered as memory T cells based on the collection time point 26 days after the last tumor had completely regressed (collection on day 62 post implantation, tumors of long-term survivors cleared by day 36, see also report for MS83: Dose-dependent efficacy of influenza virus A/CA07/09-(HA-NA)^{Min} in a murine EMT6 breast cancer model).

One key mechanism of action proposed for immunotherapeutic viruses is the induction of anti-tumor immune responses. The presence of ex vivo recall responses against virus-free tumor cell lysates suggest that influenza virus A/CA07/09-(HA-NA)^{Min} is capable of inducing durable anti-tumor immune responses that likely contribute to anti-tumor efficacy and tumor regressions. Of note, EMT6-specific responses were not significantly different between the two dose groups of influenza virus A/CA07/09-(HA-NA)^{Min}, suggesting that the treatment outcome of long-term survivorship with complete tumor regression is dependent on a certain degree of anti-tumor immune response in a given animal regardless of the required dose to induce this adequate anti-tumor immune response.

In addition to tumor antigen being released in response to treatment with influenza virus A/CA07/09-(HA-NA)^{Min}, antiviral responses were also induced. The magnitude of that response when comparing relative spot counts in long-term survivors to those in naïve control animals was lower than the anti-tumor responses (2.1-fold vs 5.2-fold in animals treated with 1×10⁸ PFU), despite the high degree of antigenicity of viral antigens as compared to most tumor antigens. While the underlying MDKC.2-specific xeno-reaction, that the ratio analysis normalizes against but is still present, may contribute to the lower magnitude as a result of antigenic competition, this data suggests that cellular immunity to influenza virus A/CA07/09-(HA-NA)^{Min} does not overshadow the anti-tumor immune responses.

Finally, IFNγ responses against MDCK.2 cell lysate were detected in animals previously treated with influenza virus A/CA07/09-(HA-NA)^{Min}. While the combined xeno-reaction to the dog cell line and any recall response to residual MDCK.2 antigen in the virus preparation led to higher IFNγ spot ratios, the relative difference between MDCK.2-specific responses in naïve control animals and influenza virus A/CA07/09-(HA-NA)^{Min}-treated animals was comparable with the EMT6-specific response. To further characterize MDCK.2-specific responses, control restimulation with lysates from a different dog cell line would be required.

In summary, this experiment provides evidence of induction of durable polyclonal immune responses after treatment with influenza virus A/CA07/09-(HA-NA)^{Min}, that are directed against both the viral agent itself as well as tumor antigens. this data is in line with the immune-stimulatory mechanisms of action that have been described for oncolytic viruses as a modality.

### Example 13

### Combination of CodaLytic with PD-1 Immune Checkpoint Inhibition in the MC38 CRC Model

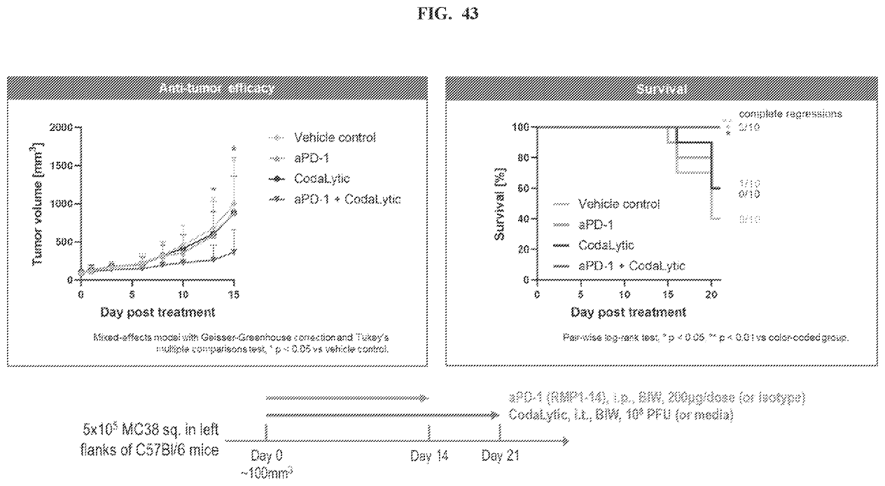

Combination of CodaLytic with aPD-1 checkpoint inhibition led to tumor growth inhibition not seen with either monotherapy. This translated into a significant survival benefit, including 30% complete regressions in an aggressive tumor model. (See FIG. 43) In most MC38 experiments, tumor growth inhibition of approx. 50% is observed with a PD-1. We used the most frequently chosen aPD-1 clone and dosing scheme for this study. Natural variability in animals studies incl. impact of exact source of animals and antibody may contribute to absence of monotherapy effect after checkpoint inhibition. The data still strongly supports the concept of combination benefit, as addition of CodaLytic to the treatment regimen led to anti-tumor efficacy.

The immune composition in splenocytes as a surrogate for peripheral immune responses was not significantly changed, as is expected for immunotherapies that target the local tumor microenvironment.

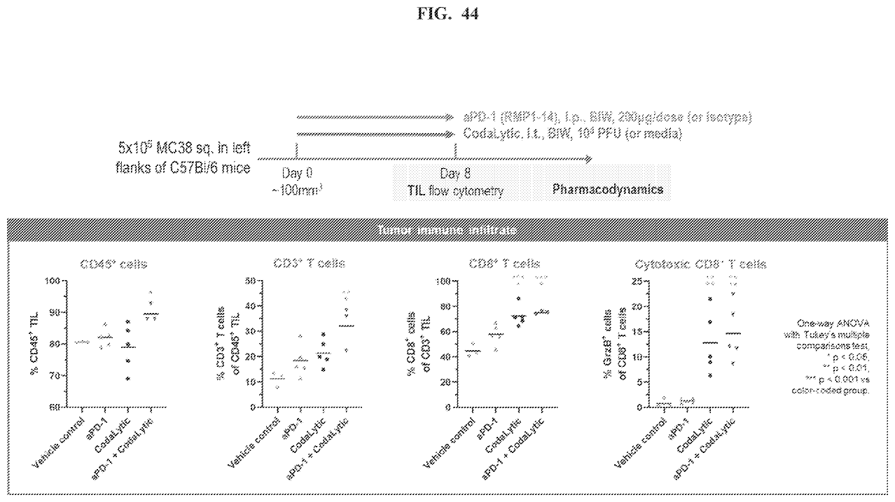

In the local tumor microenvironment, infiltration with CD8⁺ effector T cells significantly increased after combination treatment. CodaLytic treatment led to increase activation of CD8⁺ T cells, indicated by Granzyme B expression. The combination of increased amounts and improved effector status of CD8⁺ T cells appeared to correlate with efficacy. (See FIG. 44)

The influx of CD8⁺ T cells was offset by decreases in the frequency of macrophages and other myeloid cells. The CD3⁺ T cell compartment showed a relative decrease in CD4⁺ T cells, although absolute numbers of CD4⁺ T cells increased after aPD-1 treatment. This data does not allow for further dissection of subpopulation, i.e. CD4+Th1 vs Treg cells or immuno-stimulatory M1-like vs immunosuppressive M2-like macrophages. Bulk tumor RNA is available for transcriptomic analysis and potential further deconvolution of cell phenotypes.

Granzyme B as a marker of T cell activation and cytotoxicity was increased in both CD8⁺ and CD4⁺ T cells. Cytolytic CD4⁺ T cells are regularly observed and have been described as antigen-specific effectors in both infectious disease and cancer. CD44⁺ positivity was decreased after combination treatment. While this maker identifies effector and effector memory T cells, frequency of CD44⁺ T cells has also been associated with poor outcomes in cancer patients.

### Example 14A—Study 1

### CodaLytic in Therapy of 4T1 Mammary Carcinoma with Anti-PD1 and Anti-CTLA4 Antibodies

### Experimental Set Up

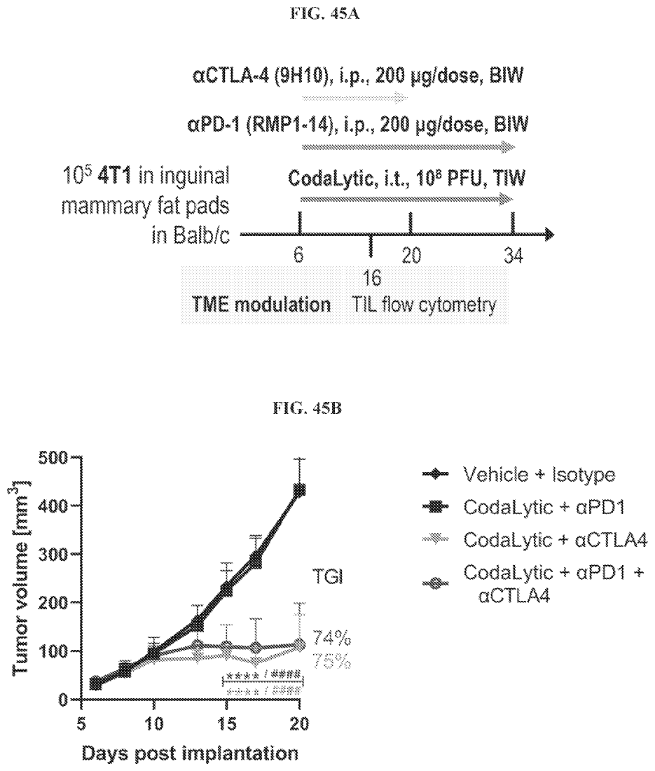

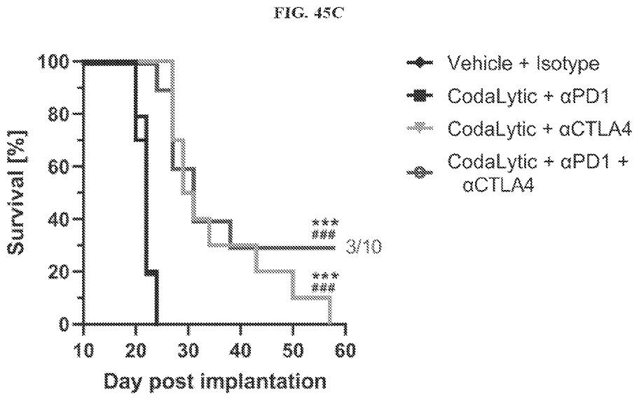

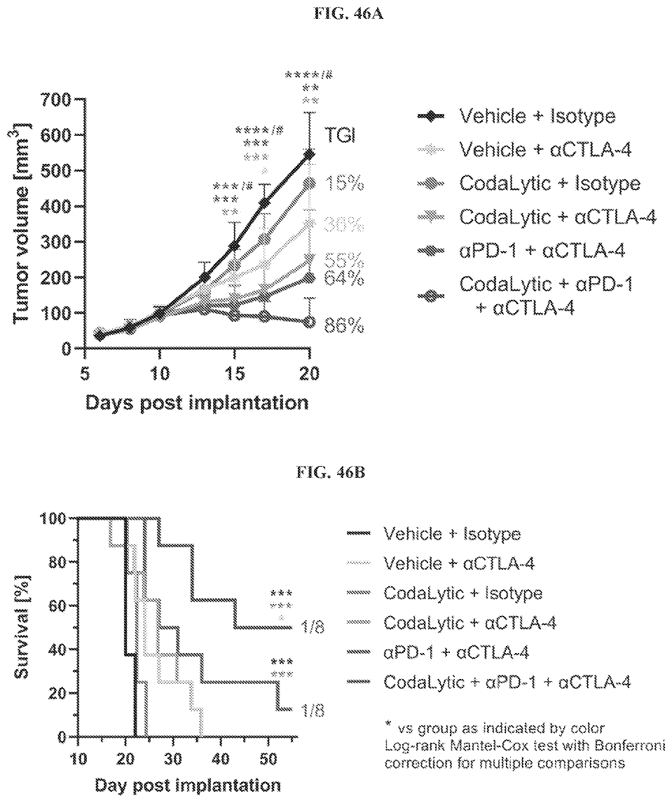

The combination of intratumoral administration of CodaLytic with systemic immune checkpoint inhibitions targeting PD-1 and CTLA-4 was tested in a TNBC model know to be refractory to PD-1 inhibition using 10 animals per treatment groups (see table below and FIG. 45A).

Dose and regimen

| | | | | Dosing | |
|---|---|---|---|---|---|
| Treatment | Dose | | Dosing Route | volume | |
| CodaLytic (3x/week for up to 4 weeks) or Vehicle | 10⁸ | PFU | I.T. | 50 | ul |
| anti-mouse PD1 (2x/week for up to 4 weeks) clone | 200 | ug | I.P. | 100 | ul |
| RMP1-14 or IgG2a isotype control | | | | | |
| anti-mouse CTLA4 (2x/week for 2 weeks) clone 9H10 | 200 | ug | I.P. | 100 | ul |
| or IgG2a isotype control | | | | | |

The final tumor size curve and long term survival curve is shown in FIGS. 45B and 45C. Combination of CodaLytic with CTLA-4 blockade significantly reduced tumor growth over time and as calculated as tumor growth inhibition on day 20 (FIG. 45B) and addition of PD-1 inhibition further improved the resulting survival benefit.

### Example 14B

### Codalytic in Therapy of 4T1 Mammary Carcinoma with Anti-PD1 and Anti-CTLA4 Antibodies-Study 2

### Experimental Set Up

The combination of intratumoral administration of CodaLytic with systemic immune checkpoint inhibitions targeting PD-1 and CTLA-4 was tested in a TNBC model know to be refractory to PD-1 inhibition using 10 animals per treatment groups (see table below and analogous to FIG. 45A). Additional control groups were included in this experiment to expand on the contribution of components as compared to Example 14A.

Dose and Regimen

| | | | Dosing | Dosing | |
|---|---|---|---|---|---|
| Treatment | Dose | | Route | volume | |
| CodaLytic (TIW for up to 4 weeks) | 10⁸ | PFU | I.T. | 50 | ul |
| anti-mouse PD1 (2x/week for up to 4 weeks) clone | 200 | ug | I.P. | 100 | ul |
| RMP1-14 or IgG2a isotype control | | | | | |
| anti-mouse CTLA4 (2x/week for 2 weeks) clone | 100 | ug | I.P. | 100 | ul |
| 9H10 or IgG2a isotype control | | | | | |

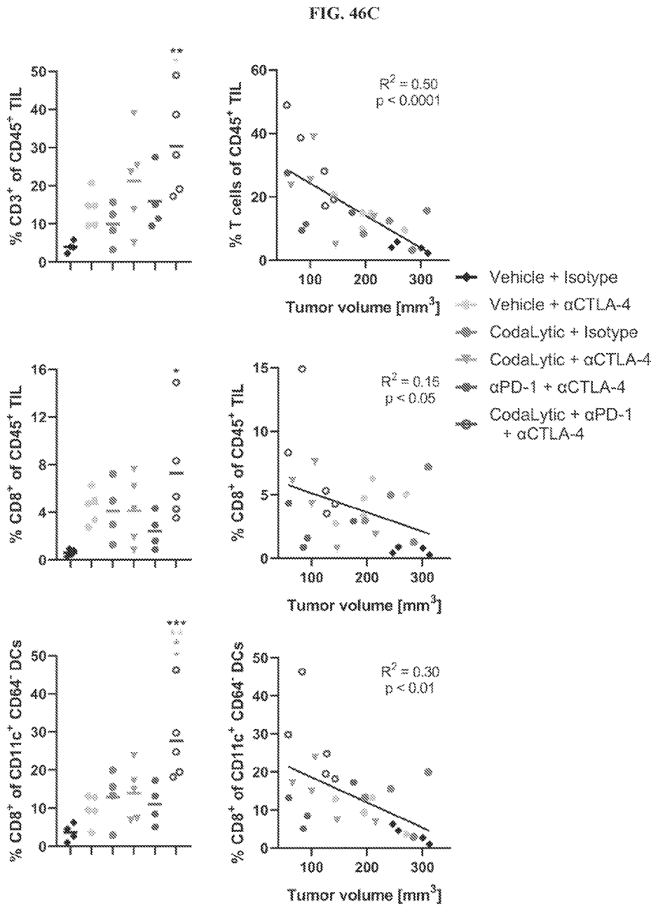

The final tumor growth curve, individual tumor size, and survival curve are shown in FIG. 46A-B, confirming the superiority of the triplet combination treatment in this PD-L1-low tumor model.

Immune cell infiltration was characterized on day 10 after treatment (see also FIG. 45A) using flow cytometry, shown in FIG. 46C. Frequency of total CD3+ T cells (top), CD8+ T cells (middle) and cross-presenting CD8+ dendritic cells (bottom) were significantly increased after triple combination therapy (2way ANOVA) and the frequency of these cell populations directly correlated with tumor volume (right).

### Example 15

### Co-Culture of Human Breast Cancer Cells with Human PBMCs

This study analyzes the infectivity of CodaLytic in human breast cancer cell lines in the presence of immune cells to mimic a tumor microenvironment, which contains a multitude of cell types. We study the viability of primary immune cells and tumor cells under different culturing conditions, infection period and MOIs.

### Experimental Design

Human Tumor cell lines: HCC1395 (ductal carcinoma, TNBC); MDA-MB-231 (adenocarcinoma, TNBC)
Human Peripheral Blood Mononuclear Cells (PBMCs): Purchased from BioIVT from healthy donors
Culturing condition: In suspension
Infection parameters: MOIs to be tested: 0, 1, 5, 10; Infection time: 24 hr, 48 hr; Seeding density: 1:1 tumor cells/PBMCs at 1×10⁶ cells/well (Total of 2×10⁶ cells/well) in 24-well plate.
Analysis method: Flow cytometry using standard protocols and antibody clones for phenotyping.

### Results

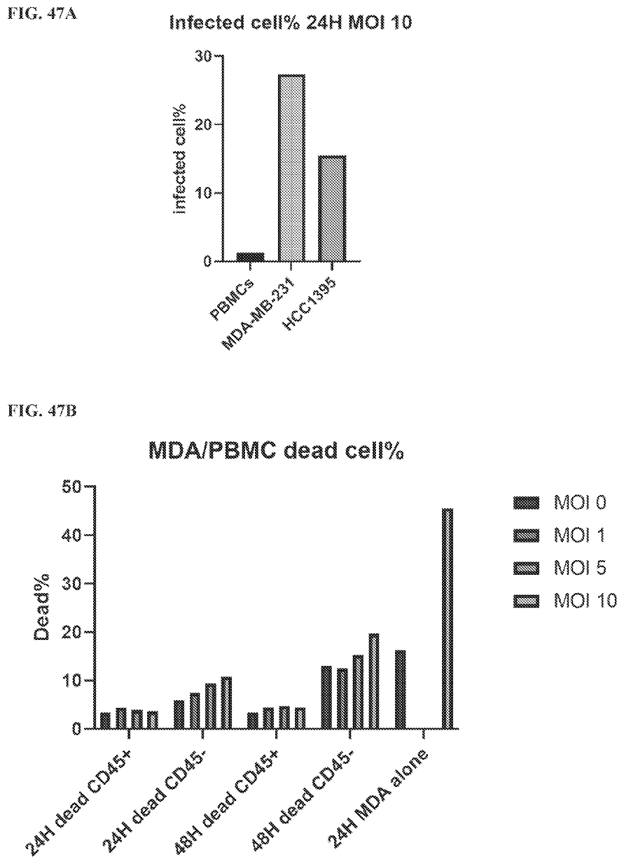

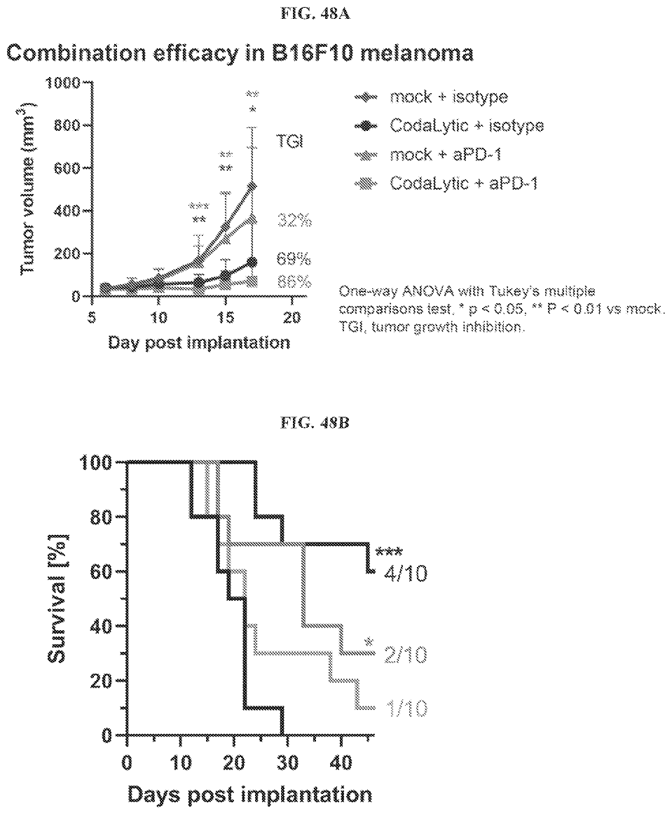

Results show a preferential infection by CodaLytic of tumor cell lines as compared to immune cells, present in human tumors to varying degrees (see FIG. 47A). CodaLytic was able to kill tumor cells to varying degrees over time, but does not kill immune cells in a time or dose-dependent manner (see FIG. 47B).

The following were observed:
Tumor cells alone get more infection than PBMCs alone at 24 h and MOI 10
Cell viability decreases with the increase of MOIs for both tumor cells and PBMCs, with relatively higher percentage observed in tumor cells
Within the immune cell compartment, viral M protein was primarily detected in B cell, DCs and monocytes, which can serve as antigen-presenting cells.
Within this compartment, CD4+ and CD8+ T cells are relatively less favored by CodaLytic infection, suggesting they may not be negatively impacted or killed by bystander infection after it. treatment with CodaLytic.

### Example 16

### Efficacy in a Gold Standard Immune-Cold Melanoma Model B16-F10

Monotherapy efficacy of CodaLytic was assessed in the well-characterized immune-resistant B16-F10 melanoma models Combination with a mouse-reactive PD-1 checkpoint inhibitor (clone RMP1-14) was also tested in the same model using efficacy and flow cytometry readouts.

FIG. 48 shows the efficacy of CodaLytic in B16F10 melanoma. 10≡B16-F10 cells were implanted subcutaneously into flanks of C57BI/6 mice and treated with CodaLytic, anti-PD-1 antibody or respective controls (Vehicle or isotype antibody) analogously to data in prior examples. Monotherapy efficacy and combination efficacy by tumor growth over time (FIG. 48A) and by survival (FIG. 48B) in B16-F10 melanoma is shown. Statistical analyses used two-way ANOVA with Tukey's multiple comparisons test and log rank test with Bonferroni correction for multiple comparisons, respectively.

### Example 17

### Additional Efficacy Data

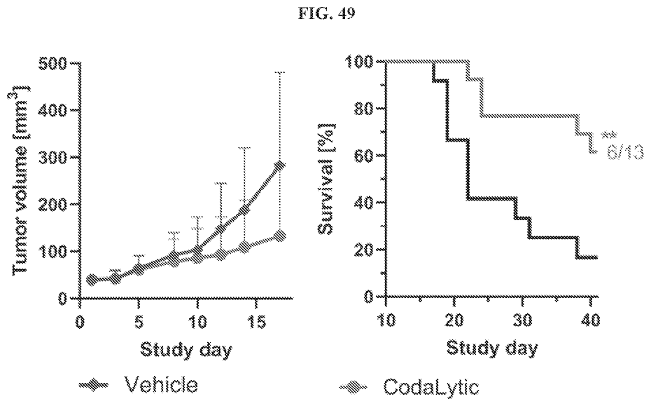

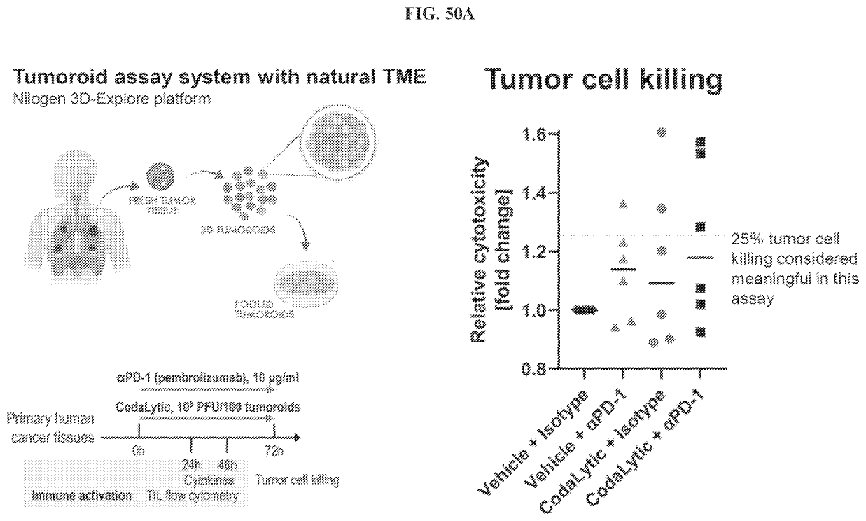

Efficacy of CodaLytic, administered in analogy to data shown above, is shown in the CT26 colon cancer model (see FIG. 49).

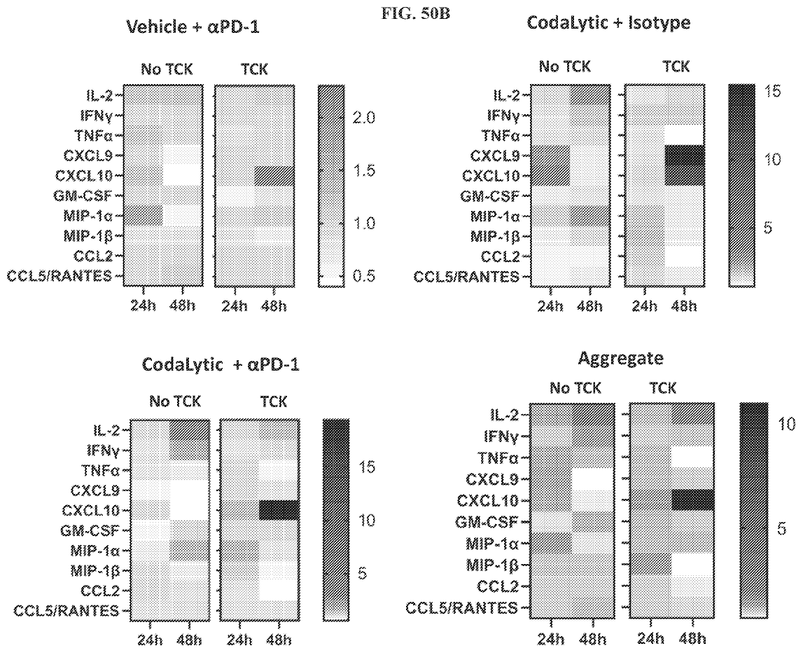

CodaLytic and CodaLytic in combination with an anti-PD-1 inhibitor pembrolizumab are also shown to be efficacious in a human tumoroid assay system with natural human tumor microenvironment (TME) (see FIG. 50). In this tumor explant model, the TME is preserved exactly as observed in cancer patients to mimic what may happen in the clinical setting, without any enzymatic manipulation or addition of autologous or ex vivo expanded immune cells. Of note, no systemic reservoir of immune cells as in a whole organism is present. As such, the model more accurately recapitulates the direct human in vivo response to CodaLytic and CodaLytic in combination with an anti-PD-1 inhibitors, and provides further confirmation of the oncolytic and short-term immunomodulatory activity.

Cytotoxicity in this model was quantified at 72 h in 100 tumoroids per source patient specimen and treatment conditions and are displayed in FIG. 50A as a fold change over the toxicity observed in Vehicle+ Isotype control treatment in each specimen.

Cytokine release was measured in supernatants taken from treated tumoroids using a Mesoscale Discovery multiplexed assay, fold changes after treatment were calculated as compared to Vehicle+ Isotype control per specimen and all conditions defined as responding conditions achieving >25% tumor cell killing or as non-responding conditions. Median fold changes are shown over time and by response status for each treatment condition and in aggregate across all treatment conditions.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) may be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein may be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Optional” or “optionally” means that the subsequently described circumstance may or may not occur, so that the description includes instances where the circumstance occurs and instances where it does not.

Groupings of alternative elements or embodiments of the present disclosure disclosed herein are not to be construed as limitations. Each group member may be referred to and claimed individually or in any combination with other members of the group or other elements found herein. One or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

1. A method of treating a malignant tumor, comprising:
administering a deoptimized influenza virus to a subject in need thereof,
wherein an HA protein of the deoptimized influenza virus is encoded by a nucleic acid having the sequence of SEQ ID NO:1, SEQ ID NO:9, SEQ ID NO:10, SEQ ID NO:13 or ORF of SEQ ID NO:13 or an HA variant of SEQ ID NO:1, SEQ ID NO:9, SEQ ID NO:10, SEQ ID NO:13 or ORF of SEQ ID NO:13 wherein the HA variant does not comprise a nucleic acid having SEQ ID NO:11 or open reading frame (ORF) of SEQ ID NO:11, and
wherein an NA protein of the deoptimized influenza virus is encoded by a nucleic acid having the sequence of SEQ ID NO:2, SEQ ID NO:14 or ORF of SEQ ID NO:14 or an NA variant of SEQ ID NO:2, SEQ ID NO:14, or ORF of SEQ ID NO:14 wherein the NA variant does not comprise a nucleic acid having SEQ ID NO:12 or ORF of SEQ ID NO:12.

2. The method of claim 1, wherein the HA protein of the deoptimized influenza virus is encoded by a nucleic acid having the sequence of SEQ ID NO:9 or SEQ ID NO:10.

3. The method of claim 1, wherein the nucleic acid sequence of the HA variant of SEQ ID NO:1, SEQ ID NO:9 or SEQ ID NO:10 comprises up to 10 mutations relative to SEQ ID NO:1, SEQ ID NO:9, or SEQ ID NO:10, respectively.

4. The method of claim 1, wherein the nucleic acid sequence of the NA variant of SEQ ID NO:2 comprises up to 10 mutations relative to SEQ ID NO:2.

5. The method of claim 1, wherein the M, PB2, PB1, PA, NS or NP protein are each encoded by a nucleic acid having SEQ ID NOs: 3, 4, 5, 6, 7, and 8, respectively, or a M, PB2, PB1, PA, NS or NP variant of SEQ ID NOs: 3, 4, 5, 6, 7, and 8, respectively.

6. The method of claim 5, wherein the M, PB2, PB1, PA, NS or NP variant of SEQ ID NOs: 3, 4, 5, 6, 7, and 8, respectively, each comprises up to 10 mutations relative to SEQ ID NOs: 3, 4, 5, 6, 7, and 8, respectively.

7. The method of claim 5, wherein the variant of SEQ ID NOs: 3, 4, 5, 6, 7, and 8, does not comprise wild-type sequence for encoding M, PB2, PB1, PA NS or NP proteins, respectively.

8. The method of claim 1, wherein the deoptimized influenza virus is administered intratumorally, subcutaneously, intramuscularly, intradermally, intranasally, or intravenously.

9. A method of treating a malignant tumor, comprising:
administering a prime dose of a deoptimized influenza virus to a subject in need thereof,
wherein an HA protein of the deoptimized influenza virus is encoded by a nucleic acid having the sequence of SEQ ID NO:1, SEQ ID NO:9, SEQ ID NO:10, SEQ ID NO:13 or ORF of SEQ ID NO:13 or an HA variant of SEQ ID NO:1, SEQ ID NO:9, SEQ ID NO:10, SEQ ID NO:13 or ORF of SEQ ID NO:13 wherein the HA variant does not comprise a nucleic acid having SEQ ID NO:11 or open reading frame (ORF) of SEQ ID NO: 11 and
wherein an NA protein of the deoptimized influenza virus is encoded by a nucleic acid having the sequence of SEQ ID NO:2, SEQ ID NO:14 or ORF of SEQ ID NO:14 or an NA variant of SEQ ID NO:2, SEQ ID NO:14, or ORF of SEQ ID NO:13 wherein the NA variant does not comprise a nucleic acid having SEQ ID NO:12 or ORF of SEQ ID NO:12; and
administering one or more boost dose of the deoptimized influenza virus to the subject in need thereof.

10-22. (canceled)

23. The method of claim 1, wherein the method further comprises administering a PD-1 inhibitor or a PD-L1 inhibitor.

24. The method of claim 23, wherein the PD-1 inhibitor is an anti-PD1 antibody, or wherein the PD-L1 inhibitor is an anti-PD-L1 antibody.

25. The method of claim 24,
wherein the anti-PD1 antibody is selected from the group consisting of pembrolizumab, nivolumab, pidilizumab, AMP-224, AMP-514, spartalizumab, cemiplimab, AGEN2034/balstilimab, AK105, BCD-100, BI 754091, JS001, LZM009, MGA012, Sym021, TSR-042/dostarlimab, MGD013, AK104, XmAb20717, tislelizumab, and combinations thereof, or
wherein the anti-PD-L1 antibody is selected from the group consisting of BGB-A333, CK-301, FAZ053, KN035, MDX-1105, MSB2311, SHR-1316, atezolizumab, avelumab, durvalumab, BMS-936559, CK-301, and combinations thereof.

26. The method of claim 23,
wherein the PD-1 inhibitor is selected from the group consisting of PF-06801591, anti-PD1 antibody expressing pluripotent killer T lymphocytes (PIK-PD-1), autologous anti-EGFRvIII 4SCAR-IgT cells, and combinations thereof, or
wherein the PD-1 inhibitor is selected from the group consisting of PF-06801591, anti-PD1 antibody expressing pluripotent killer T lymphocytes (PIK-PD-1), autologous anti-EGFRvIII 4SCAR-IgT cells, and combinations thereof, or wherein the anti-PD-L1 inhibitor is M7824.

27. (canceled)

28. (canceled)

29. (canceled)

30. The method of claim 1, further comprising administering one or more of chemotherapeutic agent, immunotherapeutic agent, anti-cancer drug, therapeutic viral particle, antimicrobial, cytokine, therapeutic protein, immunotoxin, immunosuppressant, and gene therapeutic.

31. The method of claim 1,
wherein treating the malignant tumor decreases the likelihood of recurrence of the malignant tumor, or
wherein treating the malignant tumor decreases the likelihood of having a second cancer that is different from the malignant tumor, or
wherein if the subject develops a second cancer that is different from the malignant tumor, the treatment of the malignant tumor results in slowing the growth of the second cancer,
wherein after remission of the malignant tumor, if the subject develops a second cancer that is different from the malignant tumor, the treatment of the malignant tumor results in slowing the growth of the second cancer, or
wherein treating the malignant tumor stimulates an inflammatory immune response in the tumor, or
wherein treating the malignant tumor recruits pro-inflammatory cells to the tumor, or
wherein treating the malignant tumor stimulates an anti-tumor immune response, or
wherein treating the malignant tumor reduces the tumor size.

32-38. (canceled)

39. The method of claim 1, wherein the malignant tumor is breast cancer, glioblastoma, adenocarcinoma, melanoma, lung carcinoma, neuroblastoma, bladder cancer, colon cancer, prostate cancer, or liver cancer.

40. A deoptimized influenza virus, comprising:
an HA protein encoded by a nucleic acid having the sequence of SEQ ID NO:1, SEQ ID NO:9, SEQ ID NO:10; SEQ ID NO:13 or ORF of SEQ ID NO:13 or an HA variant of SEQ ID NO:1, SEQ ID NO:9, SEQ ID NO:10, SEQ ID NO:13 or ORF of SEQ ID NO:13 wherein the HA variant does not comprise a nucleic acid having SEQ ID NO:11 or open reading frame (ORF) of SEQ ID NO:11; and
an NA protein encoded by a nucleic acid having the sequence of SEQ ID NO:2, SEQ ID NO:14 or ORF of SEQ ID NO:14 or an NA variant of SEQ ID NO:2, SEQ ID NO:14, or ORF of SEQ ID NO:14 wherein the NA variant does not comprise a nucleic acid having SEQ ID NO:12 or ORF of SEQ ID NO:12.

41. The deoptimized influenza virus of claim 40, wherein the HA protein of the deoptimized influenza virus is encoded by a nucleic acid having the sequence of SEQ ID NO:9 or SEQ ID NO:10.

42. The deoptimized influenza virus of claim 40, wherein the nucleic acid sequence of the HA variant of SEQ ID NO:1, SEQ ID NO:9 or SEQ ID NO:10 comprises up to 10 mutations relative to SEQ ID NO:1, SEQ ID NO:9, or SEQ ID NO:10, respectively.

43. The deoptimized influenza virus of claim 40, wherein the nucleic acid sequence of the NA variant of SEQ ID NO:2 comprises up to 10 mutations relative to SEQ ID NO:2.

44. The deoptimized influenza virus of claim 40, wherein the M, PB2, PB1, PA, NS or NP protein are each encoded by a nucleic acid having SEQ ID NOs: 3, 4, 5, 6, 7, and 8, respectively, or a M, PB2, PB1, PA, NS or NP variant of SEQ ID NOs: 3, 4, 5, 6, 7, and 8, respectively.

45. The deoptimized influenza virus of claim 40, wherein the M, PB2, PB1, PA, NS or NP variant of SEQ ID NOs: 3, 4, 5, 6, 7, and 8, respectively, each comprises up to 10 mutations relative to SEQ ID NOs: 3, 4, 5, 6, 7, and 8, respectively.

46. The deoptimized influenza virus of claim 40, wherein the variant of SEQ ID NOs: 3, 4, 5, 6, 7, and 8, does not comprise wild-type sequence for encoding M, PB2, PB1, PA NS or NP proteins, respectively.

47. A composition comprising the deoptimized influenza virus of any one of claim 40.

48. The composition of claim 47, wherein the composition is an immune composition.

49. The composition of claim 47, wherein the composition comprises about 10⁵-10⁹ PFU of the deoptimized influenza virus.

50. The composition of claim 47, formulated for parenteral administration, intratumor administration, intramuscular injection, subcutaneous injection, or intravenous administration.

51-53. (canceled)
